(19) United States
(12) Patent Application Publication
(10) Pub. No.: US 2025/0102497 A1
(43) Pub. Date: Mar. 27, 2025

(54) CYANINE DYES AND THEIR BIOLOGICAL CONJUGATES

(71) Applicant: AAT Bioquest, Inc., Sunnyvale, CA (US)

(72) Inventors: Zhenjun DIWU, Danville, CA (US); Guangyao GAO, Mountain View, CA (US); Jixiang LIU, Pleasanton, CA (US); Haitao GUO, Sunnyvale, CA (US)

(73) Assignee: AAT Bioquest, Inc., Sunnyvale, CA (US)

(21) Appl. No.: 18/476,278

(22) Filed: Sep. 27, 2023

(51) Int. Cl. G01N 33/542; C07D 209/14; C07D 403/06; C07D 403/14; C07F 9/6558; C07H 15/26; C07K 1/13; C12Q 1/6818; G01N 21/64; G01N 33/58

(52) U.S. Cl. CPC G01N 33/542; C07D 209/14; C07D 403/06; C07D 403/14; C07F 9/65583; C07H 15/26; C07K 1/13; C12Q 1/6818; G01N 21/6428; G01N 33/582; G01N 2021/6439; G01N 2333/95

(57) ABSTRACT

Cyanine compounds that are substantially non-fluorescent or only weakly fluorescent are useful as energy acceptors. Chemically reactive compounds possess utility for labeling a wide variety of substances, including biomolecules with the resulting conjugates being highly useful for a variety of energy-transfer assays and applications.

### SEQUENCE LISTING

The instant application contains a Sequence Listing which has been submitted electronically in XML format and is hereby incorporated by reference in its entirety. Said XML copy, created on 12 Jan. 2024, is named 36392-56663US_006US.xml and is 12,071 bytes in size.

### BACKGROUND

Fluorescence Resonance Energy Transfer (FRET) is a process whereby a first fluorescent dye (the “donor” dye) is excited, typically by illumination, and transfers its absorbed energy to a second dye (the “acceptor” dye) having a longer wavelength and therefore lower energy emission. Where the second dye is fluorescent, this energy transfer may result in fluorescence at the emission wavelength of the second dye. However, where the second dye is nonfluorescent, the absorbed energy does not result in fluorescence emission, and the fluorescence of the initial donor dye is said to be “quenched”. Energy transfer can also be utilized to quench the emission of fluorescent donors, including phosphorescent and fluorescent donors. When a fluorescent emission is restored by preventing energy transfer, the fluorescence is said to be “dequenched” or “unquenched”.

Techniques employing FRET have been utilized to study DNA hybridization and amplification, the dynamics of protein folding, proteolytic degradation, and interactions between other biomolecules (Methods in Enzymology, Vol. 278). A common donor-acceptor dye pair utilized for these applications is dabcyl(the quenching dye) and EDANS (the fluorophore). Selected examples of biological applications of FRET can be found in the following references, among others:
(1) Holskin, B. P.; Bukhtiyarova, M.; Dunn, B. M.; Baur, P.; Dechastonay, J.; Pennington, M. W. Anal Biochem 1995, 227, 148-155.
(2) Beekman, B.; Drijfhout, J. W.; Bloemhoff, W.; Ronday, H. K.; Tak, P. P.; the Koppele, J. M. FEBS Lett 1996, 390, 221-225.
(3) Pennington, M. W.; Thomberry, N. A. Peptide Research 1994, 7, 72-76.
(4) Wang, Q. M.; Johnson, R. B.; Cohen, J. D.; Voy, G. T.; Richardson, J. M.; Jungheim, L. N. Antivir Chem Chemother 1997, 8, 303-310.
(5) Guinik, S. V.; Suvorov, L. I.; Majer, P.; Collins, J.; Kane, B. P.; Johnson, D. G.; Erickson, J. W. FEBS Lett 1997, 413, 379-384.
(6) Beekman, B.; van El, B.; Drijfhout, J. W.; Ronday, H. K.; TeKoppele, J. M. FEBS Lett 1997, 418, 305-309.
(7) Beebe, K. D.; Pei, D. Anal Biochem 1998, 263, 51-56.
(8) Cook, R. M., et al., U.S. Pat. Nos. 7,109,312; 7,582,432; 8,633,307; 8,946,404; 9,139,610; 10,301,349; Diwu et al, US Pat Appl 2010/0311184.

Unfortunately, the low wavelength excitation used for the dabcyl-EDANS dye pair is not optimal due to the autofluorescence exhibited by most cellular systems, and ultraviolet light can also cause DNA cross-linking in some systems. The background is even severe with tissue samples due to a variety of colors associated with tissue samples. Many drugs, potential drugs, and biologically active proteins also have very strong absorptions in the low wavelength region. In addition, dabcyl and EDANS have low extinction coefficients, resulting in assays that are comparatively insensitive. Azo dyes are still predominantly used in a variety of FRET-based biological tests and detections (see Cook, R. M., et al., U.S. Pat. Nos. 7,109,312; 7,582,432; 8,633,307; 8,946,404; 9,139,610; 10,301,349). Even the improved analogs of dabcyl-based azo dyes still have similar limitations such as low extinction coefficients as dabcyl does (see Cook, R. M., et al., U.S. Pat. Nos. 7,109,312; 7,582,432; 8,633,307; 8,946,404; 9,139,610; 10,301,349; Diwu et al, US Pat Appl 2010/0311184).

Cyanines are generally known to be highly fluorescent with large extinction coefficients and adjustable wavelengths. Therefore, they are widely used for labeling biomolecules for developing a variety of fluorescent probes (see Waggoner, A. et al., U.S. Pat. Nos. 5,627,027; 6,048,982; 6,207,464; Pandey, R. K., et al., U.S. Pat. No. 9,821,062). However, there are very few cyanine compounds used for quenching fluorescence due to their inherently strong fluorescence. Linda et al (U.S. Pat. Nos. 6,348,596; 6,541,618; 6,750,024) disclosed a group of nitrated cyanine dyes that have reduced fluorescence. Nitro group is one of the strongest electron-withdrawing groups. Unfortunately, all the disclosed structures of nitrated cyanine dyes require the existence of at least one nitro group, making these molecules have undesired water solubility and low stability. In some cases, a single nitro group does not effectively reduce the fluorescence of a cyanine compound to be useful as a quencher due to the significant residual fluorescence (e.g., fluorescence quantum yields higher than 1%). The addition of multiple nitro groups to cyanines causes a few severe problems such as poor water solubility and low stability. In addition, these molecules are difficult to have a phosphoramidite group installed for labeling oligonucleotides due to their instability. Peng et al (U.S. Pat. No. 8,227,621) disclosed a group of alkylamino-substituted cyanine dyes for reducing their fluorescence. However, these alkylamino-substituted cyanines still have certain residual fluorescence, thus causing higher detection backgrounds. We found that arylamino-substituted and heteroarylamino-substituted cyanine molecules are non-fluorescent. Under the same conditions, arylamino and heteroarylamino substituents are much more effective groups to eliminate the fluorescence of cyanine compounds. The resulting arylamino-substituted and heteroarylamino-substituted cyanine molecules are excellent dark FRET acceptors due to their extremely broad absorption band, high extinction coefficient and non-detectable fluorescence. For the same wavelength of dyes, arylamino-substituted and heteroarylamino-substituted cyanine molecules have much higher stability. These arylamino-substituted and heteroarylamino-substituted cyanine molecules can be readily used to develop FRET-based probes for detecting protease activity, monitoring a nucleic acid target, or detecting other biological activities.

### BRIEF DESCRIPTION OF THE DRAWINGS

FIG. 1 displays a reaction scheme showing an example synthesis of a selected cyanine phosphoramidite dye.

FIG. 2 displays a reaction scheme showing an example synthesis of selected cyanine dyes conjugated to a CPG polymer support.

FIG. 3 displays an absorption spectrum of Compound 10 in water its extinction coefficient of 200,000 cm⁻¹M⁻¹. As seen from the spectra, it well overlaps with the fluorescence spectra of well-known near IR fluorescent dyes such as C5.5, Cy7, Alexa Fluor 680, Alexa Fluor 750, iFluor 680 and iFluor 750.

FIG. 4 is the absorption spectra of Compound 21 in water with its extinction coefficient of 200,000 cm⁻¹M⁻¹. As seen from the spectra, it well overlaps with the fluorescence spectra of well-known IR fluorescent dyes such as Cy7.5, Alexa Fluor 790, IRDye 800CW, IRDye 800RS, iFluor 790 and iFluor 820.

FIG. 5 is a plot showing how HIV protease-mediated cleavage of substrate peptides can be monitored via fluorescence resonance energy transfer (FRET) using cyanine dye as a dark FRET acceptor, as described in Example 45.

FIG. 6 is a plot showing how matrix metalloproteinase (MMP) mediated cleavage of a FRET substrate. FRET enzyme substrates can be monitored by fluorescence using Compound 4-gamma-Abu-Pro-Cha-Abu-Smc-His-Ala-Dab (Cy7)-Ala-Lys-NH₂ (SEQ ID NO: 1), a FRET substrate as described in Example 44. 1=MMP-1 (collagenase), 2=MMP-2 (gelatinase), 3=MMP-3 (stromelysin), 4=MMP-7 (matrilysin, PUMP-1), 5=MMP-8 (neutrophil collagenase), 6=MMP-9 (92 kDa gelatinase), 7=MMP-10 (stromelysin-2), 8=MMP-11 (stromelysin-3) and 9=MMP-12 (macrophage elastase).

### DETAILED DESCRIPTION

As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

The abbreviations used herein have their conventional meaning without the chemical and biological arts. The chemical structures and formulae set forth herein are constructed according to the standard rules of chemical valency known in the chemical arts.

Unless defined otherwise, all technical and scientific terms used herein have the same meaning as is commonly understood by one of skill in the art to which this invention belongs.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Compounds are described using standard nomenclature. The compounds in any of the formulas described herein may be in the form of a racemate, enantiomer, mixture of enantiomers, diastereomer, mixture of diastereomers, tautomer, N-oxide, isomer; such as rotamer, as if each is specifically described unless specifically excluded by context.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

A dash (“-”) that is not between two letters or symbols is used to indicate a point of attachment for a substituent. For example, —(C═O) NH₂ is attached through carbon of the carbonyl(C═O) group.

The present disclosure includes compounds (e.g., as described herein) with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorous, fluorine, chlorine and iodine such as ²H, ³H, ¹¹C, ¹³C, ¹⁴C, ¹⁵N, ¹⁸F, ³¹P, ³²P, ³⁵S, ³⁶Cl, and ¹²⁵I respectively. In one non-limiting embodiment, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in treatment of patients. In particular, an 18F labeled compound may be particularly desirable for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Isotopic substitutions, for example deuterium substitutions, can be partial or complete. Partial deuterium substitution means that at least one hydrogen is substituted with deuterium. In certain embodiments, the isotope is 90, 95, or 99% or more enriched in an isotope at any location of interest. In one non-limiting embodiment, deuterium is 90, 95, or 99% enriched at a desired location.

In some embodiments, the substitution of a hydrogen atom for a deuterium atom can be provided in any compound of Formulas I, II, III, III, IV, V, VI, VII, VIII, IX, X, or XI. In one non-limiting embodiment, the substitution of a hydrogen atom for a deuterium atom occurs within one or more groups selected from any of R₁, R₂, R₅, R₄, R₅, R₆, R₇, R₈, R₉, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R, R′, and R″ etc., For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCDa etc.). In certain other embodiments, when two substituents are combined to form a cycle the unsubstituted carbons may be deuterated.

“Aliphatic” refers to a saturated or unsaturated, straight, branched, or cyclic hydrocarbon. “Aliphatic” is intended herein to include, but is not limited to, alkyl, alkenyl, alkynyl, cycloalkyl, cycloalkenyl, and cycloalkynyl moieties, and thus incorporates each of these definitions. In one embodiment, “aliphatic” is used to indicate those aliphatic groups having 1-20 carbon atoms. The aliphatic chain can be, for example, mono-unsaturated, di-unsaturated, tri-unsaturated, or polyunsaturated, or alkynyl. Unsaturated aliphatic groups can be in a cis or trans configuration. In one embodiment, the aliphatic group contains from 1 to about 12 carbon atoms, more generally from 1 to about 6 carbon atoms or from 1 to about 4 carbon atoms. In one embodiment, the aliphatic group contains from 1 to about 8 carbon atoms. In certain embodiments, the aliphatic group is C₁-C₂, C₁-C₃, C₁-C₄, C₁-C₅ or C₁-C₆. The specified ranges as used herein indicate an aliphatic group having each member of the range described as an independent species. For example, the term C₁-C₆ aliphatic as used herein indicates a straight or branched alkyl, alkenyl, or alkynyl group having from 1, 2, 3, 4, 5, or 6 carbon atoms and is intended to mean that each of these is described as an independent species. For example, the term C₁-C₄ aliphatic as used herein indicates a straight or branched alkyl, alkenyl, or alkynyl group having from 1, 2, 3, or 4 carbon atoms and is intended to mean that each of these is described as an independent species. In one embodiment, the aliphatic group is substituted with one or more functional groups that results in the formation of a stable moiety.

“Alkyl” is a branched or straight chain saturated aliphatic hydrocarbon group. In one non-limiting embodiment, the alkyl group contains from 1 to about 12 carbon atoms, more generally from 1 to about 6 carbon atoms or from 1 to about 4 carbon atoms. In one non-limiting embodiment, the alkyl contains from 1 to about 8 carbon atoms. In certain embodiments, the alkyl is C₁-C₂, C₁-C₃, C₁-C₄, C₁-C₅, or C₁-C₆. The specified ranges as used herein indicate an alkyl group having each member of the range described as an independent species. For example, the term C₁-C₈ alkyl as used herein indicates a straight or branched alkyl group having from 1, 2, 3, 4, 5, or 6 carbon atoms and is intended to mean that each of these is described as an independent species and therefore each subset is considered separately disclosed. For example, the term C₁-C₄ alkyl as used herein indicates a straight or branched alkyl group having from 1, 2, 3, or 4 carbon atoms and is intended to mean that each of these is described as an independent species. Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, and 2,3-dimethylbutane. In an alternative embodiment, the alkyl group is optionally substituted. The term “alkyl” also encompasses cycloalkyl or carbocyclic groups. For example, when a term is used that includes “alk” then “cycloalkyl” or “carbocyclic” can be considered part of the definition, unless unambiguously excluded by the context. For example and without limitation, the terms alkyl, alkoxy, haloalkyl, etc. can all be considered to include the cyclic forms of alkyl, unless unambiguously excluded by context.

“Alkenyl” is a linear or branched aliphatic hydrocarbon groups having one or more carbon-carbon double bonds that may occur at a stable point along the chain. The specified ranges as used herein indicate an alkenyl group having each member of the range described as an independent species, as described above for the alkyl moiety. Examples of alkenyl radicals include, but are not limited to ethenyl, propenyl, allyl, propenyl, butenyl and 4-methylbutenyl. The term “alkenyl” also embodies “cis” and “trans” alkenyl geometry, or alternatively, “E” and “Z” alkenyl geometry. In an alternative embodiment, the alkenyl group is optionally substituted. The term “Alkenyl” also encompasses cycloalkyl or carbocyclic groups possessing at least one point of unsaturation.

“Alkynyl” is a branched or straight chain aliphatic hydrocarbon group having one or more carbon-carbon triple bonds that may occur at any stable point along the chain. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. In an alternative embodiment, the alkynyl group is optionally substituted. The term “Alkynyl” also encompasses cycloalkyl or carbocyclic groups possessing at least one triple bond.

“Alkylene” is a bivalent saturated hydrocarbon. Alkylenes, for example, can be a 1, 2, 3, 4, 5, 6, 7 to 8 carbon moiety, 1 to 6 carbon moiety, or an indicated number of carbon atoms, for example C₁-Czalkylene, C₁-Csalkylene, C₁-C₄alkylene, C₁-Cealkylene, or C₁-Csalkylene.

“Alkenylene” is a bivalent hydrocarbon having at least one carbon-carbon double bond. Alkenylenes, for example, can be a 2 to 8 carbon moiety, 2 to 6 carbon moiety, or an indicated number of carbon atoms, for example C₂-C₄alkenylene.

“Alkynylene” is a bivalent hydrocarbon having at least one carbon-carbon triple bond. Alkynylenes, for example, can be a 2 to 8 carbon moiety, 2 to 6 carbon moiety, or an indicated number of carbon atoms, for example C₂-C₄alkynylene.

The term “amino” refers to the group-NRR′ wherein R and R′ are independently hydrogen or nonhydrogen substituents, with nonhydrogen substituents including, for example, alkyl, aryl, alkenyl, aralkyl, and substituted and/or heteroatom-containing variants thereof.

“Chain” indicates a linear chain to which all other chains, long or short or both, may be regarded as being pendant. Where two or more chains could equally be considered to be the main chain, “chain” refers to the one which leads to the simplest representation of the molecule.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

“Halo” and “halogen” refers to fluorine, chlorine, bromine or iodine.

“Haloalkyl” is a branched or straight-chain alkyl groupssubstituted with 1 or more halo atoms described above, up to the maximum allowable number of halogen atoms. Examples of haloalkyl groups include, but are not limited to, fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Perhaloalkyl” means an alkyl group having all hydrogen atoms replaced with halogen atoms. Examples include but are not limited to, trifluoromethyl and pentafluoroethyl.

“Haloalkoxy” indicates a haloalkyl group as defined herein attached through an oxygen bridge (oxygen of an alcohol radical).

The term “heteroaliphatic” refers to an aliphatic moiety that contains at least one heteroatom in the chain, for example, an amine, carbonyl, carboxy, oxo, thio, phosphate, phosphonate, nitrogen, phosphorus, silicon, or boron atoms in place of a carbon atom. In one embodiment, the only heteroatom is nitrogen. In one embodiment, the only heteroatom is oxygen. In one embodiment, the only heteroatom is sulfur. “Heteroaliphatic” is intended herein to include, but is not limited to, heteroalkyl, heteroalkenyl, heteroalkynyl, heterocycloalkyl, heterocycloalkenyl, and heterocycloalkynyl moieties. In one embodiment, “heteroaliphatic” is used to indicate a heteroaliphatic group (cyclic, acyclic, substituted, unsubstituted, branched or unbranched) having 1-20 carbon atoms. In one embodiment, the heteroaliphatic group is optionally substituted in a manner that results in the formation of a stable moiety. Nonlimiting examples of heteroaliphatic moieties are polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

“Heterocycloalkyl” is an alkyl group as defined herein substituted with a heterocyclo group as defined herein.

“Arylalkyl” is an alkyl group as defined herein substituted with an aryl group as defined herein.

“Heteroarylalkyl” is an alkyl group as defined herein substituted with a heteroaryl group as defined herein.

The term “alkynyl” refers to a linear or branched hydrocarbon group of 2 to 24 carbon atoms containing at least one triple bond, such as ethynyl, n-propynyl, and the like. Generally, although again not necessarily, alkynyl groups herein may contain 2 to about 18 carbon atoms, and such groups may further contain 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ ary”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”, e.g., anthracyl). “Ary” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. The one or more fused carbocyclyl or heterocyclyl groups can be 4 to 7 or 5 to 7-membered saturated or partially unsaturated carbocyclyl or heterocyclyl groups that optionally contain 1, 2, or 3 heteroatoms independently selected from nitrogen, oxygen, phosphorus, sulfur, silicon and boron, to form, for example, a 3,4-methylenedioxyphenyl group. In one non-limiting embodiment, aryl groups are pendant. An example of a pendant ring is a phenyl group substituted with a phenyl group. In an alternative embodiment, the aryl group is optionally substituted as described above. In certain embodiments, the aryl group is an unsubstituted C₆₋₁₄ aryl. In certain embodiments, the aryl group is a substituted C₆₋₁₄ aryl. An aryl group may be optionally substituted with one or more functional groups that include but are not limited to, halo, hydroxy, nitro, amino, cyano, haloalkyl, aryl, heteroaryl, and heterocyclo.

The term “heterocyclyl” (or “heterocyclo”) includes saturated, and partially saturated heteroatom-containing ring radicals, where the heteroatoms may be selected from nitrogen, sulfur and oxygen. Heterocyclic rings comprise monocyclic 3-8 membered rings, as well as 5-16 membered bicyclic ring systems (which can include bridged fused and spiro-fused bicyclic ring systems). It does not include rings containing —O—O—, —O—S— or —S—S—portions. Said “heterocyclyl” group may be optionally substituted, for example, with 1, 2, 3, 4 or more substituents that include but are not limited to, hydroxyl, Boc, halo, haloalkyl, cyano, alkyl, aralkyl, oxo, alkoxy, and amino. Examples of saturated heterocyclo groups include saturated 3- to 6-membered heteromonocyclic groups containing 1 to 4 nitrogen atoms [e.g. pyrrolidinyl, imidazolidinyl, piperidinyl, pyrrolinyl, piperazinyl]; saturated 3 to 6-membered heteromonocyclic group containing 1 to 2 oxygen atoms and 1 to 3 nitrogen atoms [e.g. morpholinyl]; saturated 3 to 6-membered heteromonocyclic group containing 1 to 2 sulfur atoms and 1 to 3 nitrogen atoms [e.g., thiazolidinyl]. Examples of partially saturated heterocyclyl radicals include but are not limited to, dihydrothienyl, dihydropyranyl, dihydrofuryl, and dihydrothiazolyl. Examples of partially saturated and saturated heterocyclo groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1 \ ′-benzo[d]isothiazol-6-yl, dihydropyranyl, dihydrofuryl and dihydrothiazolyl.

Heterocyclo groups also include radicals where heterocyclic radicals are fused/condensed with aryl or heteroaryl radicals: such as unsaturated condensed heterocyclic group containing 1 to 5 nitrogen atoms, for example, indoline, isoindoline, unsaturated condensed heterocyclic group containing 1 to 2 oxygen atoms and 1 to 3 nitrogen atoms, unsaturated condensed heterocyclic group containing 1 to 2 sulfur atoms and 1 to 3 nitrogen atoms, and saturated, partially unsaturated and unsaturated condensed heterocyclic group containing 1 to 2 oxygen or sulfur atoms.

The term “heteroaryl” denotes aryl ring systems that contain one or more heteroatoms selected from O, N and S, wherein the ring nitrogen and sulfur atom(s) are optionally oxidized, and nitrogen atom(s) are optionally quarternized. Examples include but are not limited to, unsaturated 5 to 6 membered heteromonocyclyl groups containing 1 to 4 nitrogen atoms, such as pyrrolyl, imidazolyl, pyrazolyl, 2-pyridyl, 3-pyridyl, 4-pyridyl, pyrimidyl, pyrazinyl, pyridazinyl, triazolyl [e.g., 4H-1,2,4-triazolyl, IH-1,2,3-triazolyl, 2H-1,2,3-triazolyl]; unsaturated 5- to 6-membered heteromonocyclic groups containing an oxygen atom, for example, pyranyl, 2-furyl, 3-furyl, etc.; unsaturated 5 to 6-membered heteromonocyclic groups containing a sulfur atom, for example, 2-thienyl, 3-thienyl, etc.; unsaturated 5- to 6-membered heteromonocyclic groups containing 1 to 2 oxygen atoms and 1 to 3 nitrogen atoms, for example, oxazolyl, isoxazolyl, oxadiazolyl [e.g., 1,2,4-oxadiazolyl, 1,3,4-oxadiazolyl, 1,2,5-oxadiazolyl]; unsaturated 5 to 6-membered heteromonocyclic groups containing 1 to 2 sulfur atoms and 1 to 3 nitrogen atoms, for example, thiazolyl, thiadiazolyl [e.g., 1,2,4-thiadiazolyl, 1,3,4-thiadiazolyl, 1,2,5-thiadiazolyl].

The term “optionally substituted” denotes the substitution of a group herein by a moiety including, but not limited to, C₁-C₁₀ alkyl, C₂-C₁₀ alkenyl, C₂-C₁₀ alkynyl, C₃-C₁₂ cycloalkyl, C₃-C₁₂ cycloalkenyl, C₁-C₁₂ heterocycloalkyl, C₃-C₁₂ heterocycloalkenyl, C₁-C₁₀ alkoxy, aryl, aryloxy, heteroaryl, heteroaryloxy, amino, C₁-C₁₀ alkylamino, C₁-C₁₀ dialkylamino, arylamino, diarylamino, C₁-C₁₀ alkylsulfonamino, arylsulfonamino, C₁-C₁₀ alkylimino, arylimino, C₁-C₁₀ alkylsulfonimino, arylsulfonimino, hydroxyl, halo, thio, C₁-C₁₀ alkylthio, arylthio, C₁-C₁₀ alkylsulfonyl, arylsulfonyl, acylamino, aminoacyl, aminothioacyl, amidino, guanidine, ureido, cyano, nitro, azido, acyl, thioacyl, acyloxy, carboxyl, and carboxylic ester.

In one alternative embodiment any suitable group may be present on a “substituted” or “optionally substituted” position if indicated that forms a stable molecule and meets the desired purpose of the invention and includes, but is not limited to, e.g., halogen (which can independently be F, Cl, Br or I); cyano; hydroxyl; nitro; azido; alkanoyl (such as a C₂-C₈ alkanoyl group); carboxamide; alkyl, cycloalkyl, alkenyl, alkynyl, alkoxy, aryloxy such as phenoxy; thioalkyl including those having one or more thioether linkages; alkylsulfinyl; alkylsulfonyl groups including those having one or more sulfonyl linkages; aminoalkyl groups including groups having more than one N atoms; aryl (e.g., phenyl, biphenyl, naphthyl, or the like, each ring either substituted or unsubstituted); arylalkyl having for example, 1 to 3 separate or fused rings and from 6 to about 14 or 18 ring carbon atoms, with benzyl being an exemplary arylalkyl group; arylalkoxy, for example, having 1 to 3 separate or fused rings with benzyloxy being an exemplary arylalkoxy group; or a saturated or partially unsaturated heterocycle having 1 to 3 separate or fused rings with one or more N, O or S atoms, or a heteroaryl having 1 to 3 separate or fused rings with one or more N, O or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such groups may be further substituted, e.g. with hydroxy, alkyl, alkoxy, halogen and amino. In certain embodiments “optionally substituted” includes one or more substituents independently selected from halogen, hydroxyl, amino, cyano, —CHO, —COOH, —CONH₂, alkyl including C₁-C₆alkyl, alkenyl including C₂-C₆alkenyl, alkynyl including C₂-C₆alkynyl, —C₁-C₆alkoxy, alkanoyl including C₂-C₆alkanoyl, C₁-C₆alkylester, (mono- and di-C₁-C₆alkylamino) C₀-C₂alkyl, haloalkyl including C₁-C₆haloalkyl, hydoxyC₁-C₆alkyl, ester, carbamate, urea, sulfonamide, —C₁-C₆alkyl(heterocyclo), C₁-C₆alkyl(heteroaryl), —C₁-C₆alkyl(C₃-C₇cycloalkyl), O—C₁-C₆alkyl(C₃-C₇cycloalkyl), B(OH)₂, phosphate, phosphonate and haloalkoxy including C₁-C₆haloalkoxy.

When the term “substituted” appears prior or after a list of possible substituted groups, it is intended that the term apply to every member of that group. For example, the phrase “substituted alkyl and aryl” is to be interpreted as “substituted alkyl and substituted aryl.”

In addition to the disclosure herein, the term “substituted,” when used to modify a specified group or radical, can also mean that one or more hydrogen atoms of the specified group or radical are each, independently of one another, replaced with the same or different substituent groups as defined herein.

In addition to the disclosure herein, in a certain embodiment, a group that is substituted has 1, 2, 3, or 4 substituents, 1, 2, or 3 substituents, 1 or 2 substituents, or 1 substituent.

Unless indicated otherwise, the nomenclature of substituents that are not explicitly defined herein are arrived at by naming the terminal portion of the functionality followed by the adjacent functionality toward the point of attachment. For example, the substituent “hydroxyalkyl” refers to the group HO-(alkyl)-.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound.

A compound of this disclosure may form a solvate with a solvent (including water). Therefore, in one non-limiting embodiment, the present disclosure includes a solvated form of the compound. The term “solvate” refers to a molecular complex of a compound (including a salt thereof) with one or more solvent molecules. Non-limiting examples of solvents are water, ethanol, isopropanol, dimethyl sulfoxide, acetone and other common organic solvents. The term “hydrate” refers to a molecular complex comprising a compound and water. Pharmaceutically acceptable solvates in accordance with the invention include those wherein the solvent may be isotopically substituted, e.g. D₂O, d₆-acetone, d₆-DMSO. A solvate can be in a liquid or solid form.

Salts, solvates, hydrates, and prodrug forms of a compound are of interest. All such forms are embraced by the present disclosure. Thus, the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

In order to avoid the difficulties associated with the use of ultraviolet excitation, what is needed is an energy acceptor having an absorption maximum that is aligned with the emission of the fluorophore used. The energy acceptor should quench a large variety of dyes, particularly including dyes that are excited at longer wavelengths, in particular, the near infrared (IR) or infrared dyes. The compounds of the present disclosure are unique cyanine dyes that incorporate a conjugated double bond X—[CH═CH], —CH═Y, where X and Y is a 5-membered or 6-membered N-heterocycle moiety. The “H” may be replaced by other substitutes such as alkyl, aryl, halogen, thiol, amino or a heterocycle. The resulting cyanine heterocyclic dye may be substituted by a chemically reactive moiety (RM), a solid support (such as CPG) or a biological substance (such as peptides, proteins, oligonucleotides, nucleic acids or carbohydrates etc).

### Compounds

This disclosure provides cyanine heterocyclic compounds of extremely low fluorescence or no fluorescence that have been discovered to quench the fluorescence of a large variety of dyes, including dyes that are excited in the near infra-red and infra-red range, such as Cy7, Cy7.5, Alexa Fluor 790, IRDye 800CW, IRDye 800RS, iFluor 790, iFluor 820 and allophycocyanin. In addition, the compounds of the disclosure can have significantly larger extinction coefficients than the existing quenching compounds that are currently used in energy transfer assays. In contrast to conventional cyanine dyes are of strong fluorescence, the present disclosure provides particular dyes that are substantially non-fluorescent, or only weakly fluorescent. These cyanine compounds therefore represent a new and useful class of dark energy acceptors, including chemically reactive versions, and conjugates prepared therefrom.

In one aspect, the compounds of the present disclosure may be described by Formula I:

WSM−{X—[CR₁═CR₂]ₙ—CR₃═Y}—RM Formula I

wherein n is an integer from 0 to 3; X is independently an indolium, a pyrrolium, an oxazolium, a thiazolium, an imidazolium, a pyridinium, or a quinolinium moiety; Y is independently an indoline, a pyrrole, an oxazoline, a thiazoline, an imidazoline, a pyridine, or a quinoline moiety; R₁ to R₃ are independently H, an alkyl, an aryl, a halogen, a thiol, an amino or a heterocycle; RM is a chemically reactive moiety; WSM is a water-soluble moiety, provided that at least one of X and Y contains an arylamino or a heteroarylamino group.
In another aspect of Formula I, X is an indolium or a quinolinium moiety; Y is an indoline or a quinoline moiety; R₁ to R₃ are H; RM is a chemically reactive moiety; and WSM is a sulfonate or phosphonate.

In another aspect of Formula I, X is of Formula II

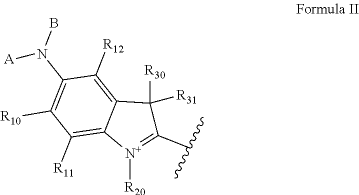

wherein A and B are independently H, an alkyl, an aryl or a heteroaryl; R₁₀ to R₁₂ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; R₂₀ is an alkyl, an aryl or a heteroaryl; R₃₀ and R₃₁ are independently an alkyl, an aryl or a heteroaryl provided that at least one of A and B is an aryl or a heteroaryl.

In another aspect of Formula II, A and B are independently H, an alkyl, or a phenyl; R₁₀ to R₁₂ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, an aryl, a substituted aryl, a heteroaryl, or a substituted heteroaryl; R₂₀ is an alkyl, an aryl or a heteroaryl; and R₃₀ and R₃₁ are independently an alkyl or an aryl.

In another aspect of Formula II, A and B are independently H, a sulfonated alkyl, or a sulfonated phenyl; R₁₀ to R₁₂ are independently H, a halogen, carboxy, an alkyl, an alkoxy, an aryloxy, an aryl, a heteroaryl; R₂₀ is an alkyl, a sulfonated alkyl or a carboxylated alkyl; R₃₀ and R₃₁ are an alkyl.

In another aspect of Formula I, X is of Formula III

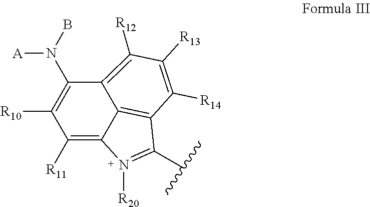

wherein A and B are independently H, an alkyl, an aryl or a heteroaryl; R₁₀ to R₁₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; and R₂₀ is an alkyl, an aryl or a heteroaryl provided that at least one of A and B is an aryl or a heteroaryl.

In another aspect of Formula II, A and B are independently H, an alkyl, or a phenyl; R₁₀ to R₁₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, an aryl, a substituted aryl, a heteroaryl, or a substituted heteroaryl; and R₂₀ is an alkyl, an aryl or a heteroaryl.

In another aspect of Formula II, A and B are independently H, a sulfonated alkyl, or a sulfonated phenyl; R₁₀ to R₁₄ are independently H, a halogen, carboxy, an alkyl, an alkoxy, an aryloxy, an aryl, a heteroaryl; and R₂₀ is an alkyl, a sulfonated alkyl or a carboxylated alkyl.

In one aspect, the compounds of the disclosure may be described by Formula XI:

{X—[CR₁═CR₂]ₙ—CR₃═Y]—CPG Formula IV

wherein n is an integer from 0 to 3; X is independently an indolium, a pyrrolium, an oxazolium, a thiazolium, an imidazolium, a pyridinium, or a quinolinium moiety; Y is independently an indoline, a pyrrole, an oxazoline, a thiazoline, an imidazoline, a pyridine, or a quinoline moiety; R₁ to R₃ are independently H, an alkyl, an aryl, a halogen, a thiol, an amino or a heterocycle; RM is a chemically reactive moiety; and CPG is a controlled pore glass as solid support for oligo nucleotide synthesis, provided that at least one of X and Y contains an arylamino or a heteroarylamino group.

In another aspect of Formula IV wherein X is an indolium or a quinolinium moiety; Y is an indoline or a quinoline moiety; R₁ to R₃ are H.

In another aspect of Formula IV, X has Formula II

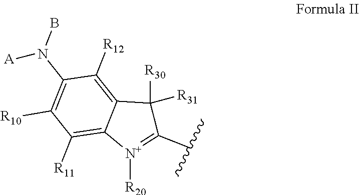

wherein A and B are independently H, an alkyl, an aryl or a heteroaryl; R₁₀ to R₁₂ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; R₂₀ is an alkyl, an aryl or a heteroaryl; and R₃₀ and R₃₁ are independently an alkyl, an aryl or a heteroaryl provided that at least one of A and B is an aryl or a heteroaryl, or an aryl.

In In another aspect of Formula II, A and B are independently H, an alkyl, or a phenyl; R₁₀ to R₁₂ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, an aryl, a substituted aryl, a heteroaryl, or a substituted heteroaryl; R₂₀ is an alkyl, an aryl or a heteroaryl; and R₃₀ and R₃ are independently an alkyl or an aryl.

In another aspect of Formula IV, X has Formula III

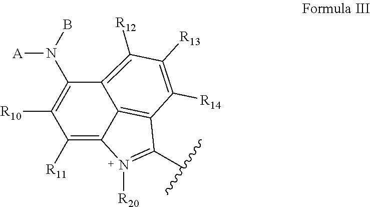

wherein A and B are independently H, an alkyl, an aryl or a heteroaryl; R₁₀ to R₁₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; and R₂₀ is an alkyl, an aryl or a heteroaryl provided that at least one of A and B is an aryl or a heteroaryl.

In another aspect of Formula II, A and B are independently H, an alkyl, or a phenyl; R₁₀ to R₁₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, an aryl, a substituted aryl, a heteroaryl, or a substituted heteroaryl; R₂₀ is an alkyl, an aryl or a heteroaryl.

In yet another aspect of this disclosure, the compounds of the disclosure may be described by Formula V:

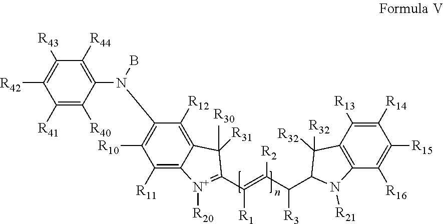

wherein B is H, an alkyl, an aryl or a heteroaryl; n is an integer from 0 to 3; R₁ to R₃ are independently H, an alkyl, an aryl, a halogen, a thiol, an amino or a heterocycle; R₁₀ to R₁₆ and Rao to R₄₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; R₂₀ and R₂₁ are independently an alkyl, an aryl or a heteroaryl; and R₃₀ to R₃₃ are independently an alkyl, an aryl or a heteroaryl, provided that at least there is a reactive moiety and a water soluble moiety.

In another aspect of Formula V, B is H, an alkyl, or a phenyl; n is an integer from 1 to 3; R₁ to R₃ are H; R₁₀ to R₁₆ and R₄₀ to R₄₄ are independently H, a halogen, carboxy, substituted carboxy, an alkyl, a substituted alkyl, an alkoxy, a substituted alkoxy, an aryloxy, a substituted aryloxy, a sulfonate, thiol, an alkylthiol, an arylthiol, an aryl, a substituted aryl, a heteroaryl, or a substituted heteroaryl; R₂₀ and R₂₁ are independently an alkyl, a carboxylated alkyl or a sulfonated alkyl; and R₃₀ to R₃₃ are an alkyl, provided that at least there are 2 sulfonates and a reactive moiety.

In some embodiments, each of R₁, R₂, and R₃ are independently selected from H, halogen,

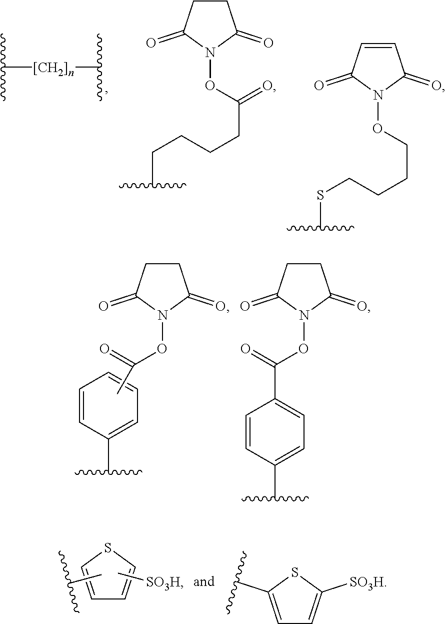

In another aspect of Formula V, B is H, an alkyl, or a sulfonated alkyl; n is 1 or 2; R₁ to R₃ are H; R₁₀ to R₁₆ and R₄₀ to R₄₄ are independently H, a halogen, an alkyl, an alkoxy, a sulfonate, an aryl, or a heteroaryl; R₂₀ and R₂₁ are independently an alkyl, a carboxylated alkyl or a sulfonated alkyl; and R₃₀ to R₃₃ are methyl, provided that at least there are 2 sulfonates and a reactive moiety.

In yet another aspect of this disclosure, the compounds of the disclosure may be described by Formula VI:

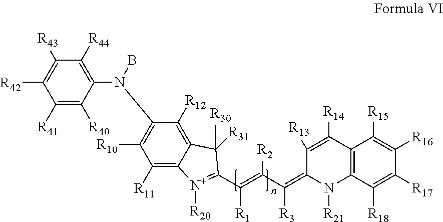

wherein B is H, an alkyl, an aryl or a heteroaryl; n is an integer from 0 to 3; R₁ to R₃ are independently H, an alkyl, an aryl, a halogen, a thiol, an amino or a heterocycle; R₁₀ to R₁₈ and R₄₀ to R₄₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; R₂₀ and R₂₁ are independently an alkyl, an aryl or a heteroaryl; and R₃₀ and R₃₁ are independently an alkyl, an aryl or a heteroaryl, provided that at least there is a reactive moiety and a water soluble moiety.

In another aspect of Formula VI, B is H, an alkyl, or a phenyl; n is an integer from 1 to 3; R₁ to R₃ are H; R₁₀ to R₁₈ and R₄₀ to R₄₄ are independently H, a halogen, carboxy, substituted carboxy, an alkyl, a substituted alkyl, an alkoxy, a substituted alkoxy, an aryloxy, a substituted aryloxy, a sulfonate, thiol, an alkylthiol, an arylthiol, an aryl, a substituted aryl, a heteroaryl, or a substituted heteroaryl; R₂₀ and R₂₁ are independently an alkyl, a carboxylated alkyl or a sulfonated alkyl; and R₃₀ and R₃₁ are an alkyl, provided that at least there are 2 sulfonates and a reactive moiety.

In another aspect of Formula V, B is H, an alkyl, or a sulfonated alkyl; n is 1 or 2; R₁ to R₃ are H; R₁₀ to R₁₈ and R₄₀ to R₄₄ are independently H, a halogen, an alkyl, an alkoxy, a sulfonate, an aryl, or a heteroaryl; R₂₀ and R₂₁ are independently an alkyl, a carboxylated alkyl or a sulfonated alkyl; and R₃₀ and R₃₁ are methyl, provided that at least there are 2 sulfonates and a reactive moiety.

In yet another aspect of this disclosure, the compounds of the disclosure may be described by Formula VII:

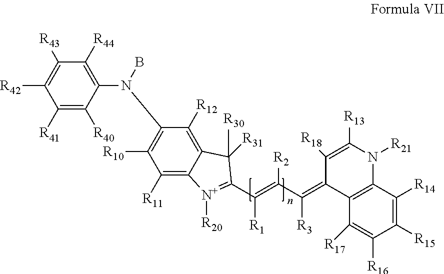

wherein B is H, an alkyl, an aryl or a heteroaryl; n is an integer from 0 to 3; R₁ to R₃ are independently H, an alkyl, an aryl, a halogen, a thiol, an amino or a heterocycle; R₁₀ to R₁₈ and R₄ to R₄₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; R₂₀ and R₂₁ are independently an alkyl, an aryl or a heteroaryl; and Rao and R₃₃ are independently an alkyl, an aryl or a heteroaryl, provided that at least there is a reactive moiety and a water soluble moiety.

In another aspect of Formula VI, B is H, an alkyl, or a phenyl; n is an integer from 1 to 3; R₁ to R₃ are H; R₁₀ to R₁₇ and R₄₀ to R₄₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, a sulfonate, an aryl, a substituted aryl, a heteroaryl, or a substituted heteroaryl; R₂₀ and R₂₁ are independently an alkyl, a carboxylated alkyl or a sulfonated alkyl; and R₃₀ and R₃₁ are an alkyl, provided that at least there are 2 sulfonates and a reactive moiety.

In another aspect of Formula V, B is H, an alkyl, or a sulfonated alkyl; n is 1 or 2; R₁ to R₃ are H; R₁₀ to R₁₈ and R₄₀ to R₄₄ are independently H, a halogen, an alkyl, an alkoxy, a sulfonate, an aryl, or a heteroaryl; R₂₀ and R₂₁ are independently an alkyl, a carboxylated alkyl or a sulfonated alkyl; and R₃₀ and R₃₁ are methyl, provided that at least there are 2 sulfonates and a reactive moiety.

In yet another aspect of this disclosure, the compounds of the disclosure may be described by Formula VIII:

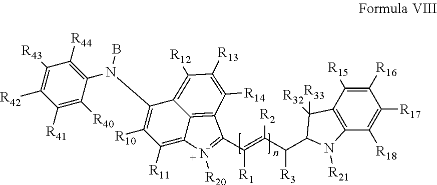

wherein B is H, an alkyl, an aryl or a heteroaryl; n is an integer from 0 to 3; R₁ to R₃ are independently H, an alkyl, an aryl, a halogen, a thiol, an amino or a heterocycle; R₁₀ to R₁₈ and R₄₀ to R₄₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; R₂₀ and R₂₁ are independently an alkyl, an aryl or heteroaryl; and R₃₂ and R₃₃ are independently an alkyl, an aryl or a heteroaryl, provided that at least there is a reactive moiety and a water soluble moiety.

In another aspect of Formula VIII, B is H, an alkyl, or a phenyl; n is an integer from 1 to 3; R₁ to R₃ are H; R₁₀ to R₁₈ and R₄₀ to R₄₄ are independently H, a halogen, carboxy, substituted carboxy, an alkyl, a substituted alkyl, an alkoxy, a substituted alkoxy, an aryloxy, a substituted aryloxy, a sulfonate, thiol, an alkylthiol, an arylthiol, an aryl, a substituted aryl, a heteroaryl, or a substituted heteroaryl; R₂₀ and R₂₁ are independently an alkyl, a carboxylated alkyl or a sulfonated alkyl; and R₃₂ and R₃₃ are an alkyl, provided that at least there are 2 sulfonates and a reactive moiety.

In another aspect of Formula VIII, B is H, an alkyl, or a sulfonated alkyl; n is 1 or 2; R₁ to R₃ are H; R₁₀ to R₁₈ and R₄₀ to R₄₄ are independently H, a halogen, an alkyl, an alkoxy, a sulfonate, an aryl, or a heteroaryl; R₂₀ and R₂₁ are independently a carboxylated alkyl or a sulfonated alkyl; and R₃₂ and R₃₃ are methyl, provided that at least there are 2 sulfonates and a reactive moiety.

In yet another aspect of this disclosure, the compounds of the disclosure may be described by Formula IX:

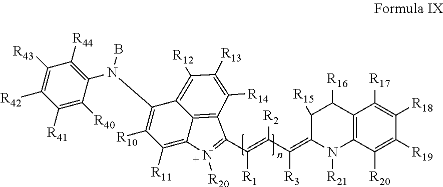

wherein B is H, an alkyl, an aryl or a heteroaryl; n is an integer from 0 to 3; R₁ to R₃ are independently H, an alkyl, an aryl, a halogen, a thiol, an amino or a heterocycle; R₁₀ to R₂₀ and R₄₀ to R₄₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; and R₂₀ and R₂₁ are independently an alkyl, an aryl or heteroaryl, provided that at least there is a reactive moiety.

In another aspect of Formula IX, B is H, an alkyl, or a phenyl; n is an integer from 1 to 3; R₁ to R₃ are H; R₁₀ to R₂₀ and R₄₀ to R₄₄ are independently H, a halogen, carboxy, substituted carboxy, an alkyl, a substituted alkyl, an alkoxy, a substituted alkoxy, an aryloxy, a substituted aryloxy, a sulfonate, thiol, an alkylthiol, an arylthiol, an aryl, a substituted aryl, a heteroaryl, or a substituted heteroaryl; and R₂₁ and R₂₂ are independently an alkyl, a carboxylated alkyl or a sulfonated alkyl, provided that at least there is a reactive moiety.

In another aspect of Formula IX, B is H, an alkyl, or a sulfonated alkyl; n is 1 or 2; R₁ to R₃ are H; R₁₀ to R₂₀ and R₄₀ to R₄₄ are independently H, a halogen, an alkyl, an alkoxy, a sulfonate, an aryl, or a heteroaryl; and R₂₁ and R₂₂ are independently a carboxylated alkyl or a sulfonated alkyl, provided that at least there are 2 sulfonates and a reactive moiety.

In yet another aspect of this disclosure, the compounds of the disclosure may be described by Formula X:

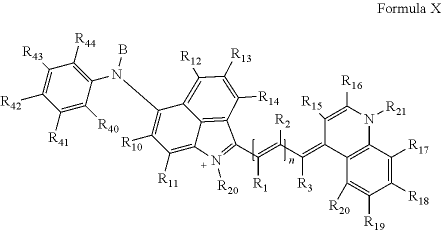

wherein B is H, an alkyl, an aryl or a heteroaryl; n is an integer from 0 to 3; R₁ to R₃ are independently H, an alkyl, an aryl, a halogen, a thiol, an amino or a heterocycle; R₁₀ to R₂₀ and R₄₀ to R₄₄ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, hydroxy, phosphonyl, sulfonyl, carbonyl, boronic acid, an aryl, a substituted aryl, a heteroaryl, or a substituted heteroaryl; and R₂₁ and R₂₂ are independently an alkyl, an aryl or a heteroaryl, provided that at least there is a reactive moiety.

In another aspect of Formula X, B is H, an alkyl, or a phenyl; n is an integer from 1 to 3; R₁ to R₃ are H; R₁₀ to R₂₀ and R₄₀ to R₄₄ are independently H, a halogen, carboxy, substituted carboxy, an alkyl, a substituted alkyl, an alkoxy, a substituted alkoxy, an aryloxy, a substituted aryloxy, a sulfonate, thiol, an alkylthiol, an arylthiol, an aryl, a substituted aryl, a heteroaryl, or a substituted heteroaryl; and R₂₁ and R₂₂ are independently an alkyl, a carboxylated alkyl or a sulfonated alkyl, provided that at least there are 2 sulfonates and a reactive moiety.

In another aspect of Formula X, B is H, an alkyl, or a sulfonated alkyl; n is 1 or 2; R₁ to R₃ are H; R₁₀ to R₂₀ and R₄₀ to R₄₄ are independently H, a halogen, an alkyl, an alkoxy, a sulfonate, an aryl, or a heteroaryl; and R₂₁ and R₂₂ are independently a carboxylated alkyl or a sulfonated alkyl.

In one aspect, the compounds of the disclosure may be described by Formula XI:

{X—[CR₁═CR₂]ₙ—CR₃═Y]—CEP Formula XI

wherein n is an integer from 0 to 3; X is independently an indolium, a pyrrolium, an oxazolium, a thiazolium, an imidazolium, a pyridinium, or a quinolinium moiety; Y is independently an indoline, a pyrrole, an oxazoline, a thiazoline, an imidazoline, a pyridine, or a quinoline moiety; R₁ to R₃ are independently H, an alkyl, an aryl, a halogen, a thiol, an amino or a heterocycle; RM is a chemically reactive moiety; and CEP is a phosphoramidite moiety, provided that at least one of X and Y contains an arylamino or a heteroarylamino group.

In another aspect of Formula XI, X is an indolium or a quinolinium moiety; Y is an indoline or a quinoline moiety; and R₁ to R₃ are H.

In another aspect of Formula XI, X is of Formula II

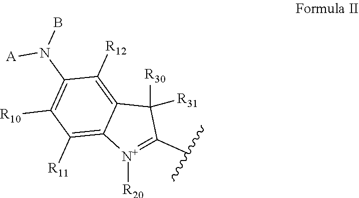

wherein A and B are independently H, an alkyl, an aryl or a heteroaryl; R₁₀ to R₁₂ are independently H, a halogen, carboxy, substituted carboxy, an alkyl, a substituted alkyl, an alkoxy, a substituted alkoxy, an aryloxy, a substituted aryloxy, thiol, an alkylthiol, an arylthiol, azido, cyano, an amino, a substituted amino, an aryl, a substituted aryl, a heteroaryl, or a substituted heteroaryl; R₂₀ is an alkyl, an aryl or a heteroaryl; and R₃₀ and R₃₁ are independently an alkyl, an aryl or a heteroaryl provided that at least one of A and B is an aryl or a heteroaryl.

In another aspect of Formula II, A and B are independently H, an alkyl, or a phenyl; R₁₀ to R₁₂ are independently H, a halogen, an alkyl, a substituted alkyl, an alkoxy, a substituted alkoxy, an aryloxy, a substituted aryloxy, an arylthiol, an aryl, a substituted aryl, a heteroaryl, or a substituted heteroaryl; R₂₀ is an alkyl, an aryl or a heteroaryl; and R₃₀ and R₃₁ are independently an alkyl or an aryl.

In another aspect of Formula XI, X has Formula III

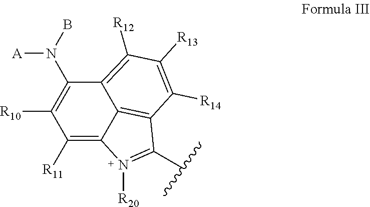

wherein A and B are independently H, an alkyl, an aryl or a heteroaryl; R₁₀ to R₁₄ are independently H, a halogen, an alkyl, a substituted alkyl, an alkoxy, a substituted alkoxy, an aryloxy, a substituted aryloxy, an alkylthiol, an arylthiol, azido, cyano, an amino, a substituted amino, an aryl, a substituted aryl, a heteroaryl, or a substituted heteroaryl; and R₂₀ is an alkyl, an aryl or a heteroaryl provided that at least one of A and B is an aryl or a heteroaryl.

In another aspect of Formula III, A and B are independently H, an alkyl, or a phenyl; R₁₀ to Ria are independently H, a halogen, an alkyl, a substituted alkyl, an alkoxy, a substituted alkoxy, an aryloxy, a substituted aryloxy, an alkylthiol, an arylthiol, an aryl, a substituted aryl, a heteroaryl, or a substituted heteroaryl; and R₂₀ is an alkyl, an aryl or a heteroaryl.

As used herein, the term “arylamino” is meant to contain the following structure:

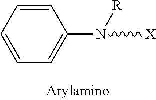

### or Y

As used herein, the term “heteroarylamino” is meant to contain the following structure:

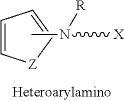

or Y (Z═CH═N, N, O, S, Se, etc)

As used herein, the term “indolium” is mean to contain the following structure:

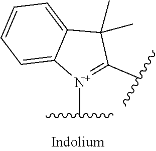 [SMILES: *C1=[N+](*)c2ccccc2C1(C)C]

As used herein, the term “pyrrolium” is meant to contain the following structure:

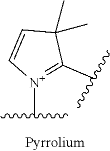 [SMILES: *C1=[N+](*)C=CC1(C)C]

As used herein, the term “oxazolium” is meant to contain the following structure:

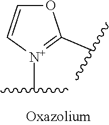 [SMILES: *c1occ[n+]1*]

As used herein, the term “thiazolium” is meant to contain the following structure:

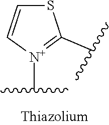 [SMILES: *c1scc[n+]1*]

As used herein, the term “imidazolium” is meant to contain the following structure:

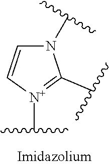 [SMILES: *c1n(*)cc[n+]1*]

As used herein, the term “pyridinium” is meant to contain the following structure:

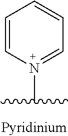

As used herein, the term “quinolinium” is meant to contain the following structure:

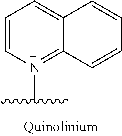

As used herein, the term “indoline” is meant to contain the following structure:

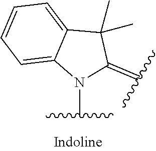 [SMILES: *N1C(=*)C(C)(C)c2ccccc21]

As used herein, the term “pyrrole” is meant to contain the following structure:

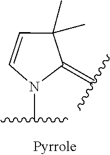 [SMILES: *N1C=CC(C)(C)C1=*]

As used herein, the term “oxazoline” is meant to contain the following structure:

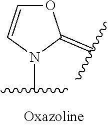 [SMILES: *N1C=COC1=*]

As used herein, the term “thiazoline” is meant to contain the following structure:

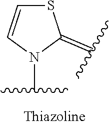 [SMILES: *N1C=CSC1=*]

As used herein, the term “imidazoline” is meant to contain the following structure:

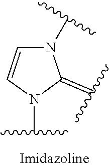 [SMILES: *N1C=CN(*)C1=*]

As used herein, the term “pyridine” is meant to contain the following structure:

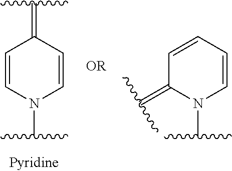 [SMILES: *N1C=CC(=*)C=C1 ; *N1C=CC=CC1=*]

As used herein, the term “quinoline” is meant to contain the following structure:

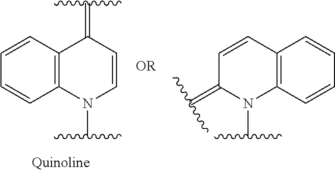 [SMILES: *N1C=CC(=*)c2ccccc21 ; *N1C(=*)C=Cc2ccccc21]

As used herein, by “sulfo” or “sulfonated” is meant sulfonic acid, or salt of sulfonic acid (sulfonate). Similarly, by “carboxy” is meant carboxylic acid or salt of carboxylic acid. “Sulfonated”, as used herein, is a moiety that contains a sulfo group. “Carboxylated”, as used herein, is a moiety that contains a carboxy group. “Phosphate”, as used herein, is an ester of phosphoric acid, and includes salts of phosphate. “Phosphonate”, as used herein, means phosphonic acid and includes salts of phosphonate. As used herein, unless otherwise specified, the alkyl portions of substituents such as alkyl, alkoxy, arylalkyl, alylamino, dialkylamino, trialkylammonium, or perfluoroalkyl are optionally saturated, unsaturated, linear or branched, and all alkyl, alkoxy, alkylamino, and dialkylamino substituents are themselves optionally further substituted by carboxy, sulfo, amino, or hydroxy.

As used herein, by “counterions” is meant the necessary ion to balance the charge of the cyanine compounds of the disclosure. Many embodiments of the compounds of the disclosure possess an overall electronic charge. It is to be understood that when such electronic charges are shown to be present, they are balanced by the presence of one or more appropriate counterions, which may or may not be explicitly identified. In some aspects, the appropriate counterion is a biologically compatible counterion. As used herein, a biologically compatible counterin is not toxic in biological applications and does not have a substantially deleterious effect on biomolecules. Where the compound of the disclosure is positively charged, the counterion is typically selected from, but not limited to, chloride, bromide, lodide, sulfate, alkanesulfonate, arylsulfonate, phosphate, perchlorate, tetrafluoroborate, tetraarylboride, nitrate, and anions of aromatic or aliphatic carboxylic acids. Where the compound of the disclosure is negatively charged, the counterion is typically selected from, but not limited to, alkali metal ions, alkaline earth metal ions, transition metal ions, ammonium or substituted ammonium ions, or pyridinium ions. Preferably, any necessary counterion is biologically compatible, is not toxic as used, and does not have a substantially deleterious effect on biomolecules. Counterions are readily changed by methods well known in the art, such as ion-exchange chromatography, or via selective precipitation.

The compounds of the disclosure may be represented by chemical formulae that represent one or another particular electronic resonance structures or another tautomerized structure. It is understood that every aspect of the description of the compounds of the disclosure applies equally to compounds that are related as formal resonance structures, as the electronic charge on the subject dyes are typically delocalized throughout the compound. Additionally, and without wishing to be bound by any particular theory, the distribution of pi electrons throughout an aromatic or molecule of extended conjugation may result in oscillations in conformations between cis and trans alkenes, formal assignments of aromaticity and formal charge assignments. Furthermore, those of skill in the art will recognize these features of multifarious chemical structure as necessary in the performance of compounds of the instant disclosure.

As used herein, by “reactive moiety (RM)” is a functional group that enables the cyanine compounds of the disclosure to conjugate with a biological substance. In one aspect, the compounds of the disclosure include at least one chemically reactive moiety (RM). The RM is chemically reactive functional group selected to cross-react with one or more types of functional groups to form a covalent bond or other linkage, typically creating a dye-conjugate.

The RM is attached to the compound of the disclosure by a covalent linkage L. L may be a single covalent bond, or the covalent linkage L may include multiple intervening atoms forming the covalent linkage. In one aspect, the covalent linkage L includes sufficient intervening atoms to serve as a spacer between the compound of the disclosure and the substance it is conjugated with. Typically, the conjugation reaction between the reactive compound and the substance to be conjugated results in one or more atoms of the reactive group RM becoming incorporated into a new linkage L′ attaching the compound to the conjugated substance.

Those compounds that include an RM are capable of labeling a wide variety of organic or inorganic substances that contain or are modified to contain functional groups with suitable reactivity, resulting in chemical attachment of the conjugated substance. Typically the reactive group is an electrophile or nucleophile that can form a covalent linkage through exposure to the corresponding functional group that is a nucleophile or electrophile, respectively. Alternatively, the reactive group is a photoactivatable group, and becomes chemically reactive only after illumination with light of an appropriate wavelength.

Selected examples of reactive groups and linkages are shown in Table 1, where the reaction of an electrophilic group and a nucleophilic group yields a covalent linkage.

TABLE 1
Examples of RM groups to useful covalent linkages

| Electrophilic Group | Nucleophilic Group | Resulting Conjugate |
|---|---|---|
| activated esters* | amines/anilines | carboxamides |
| acrylamides | thiols | thioethers |
| acyl azides** | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

*Activated esters, as understood in the art, generally have the formula —COW, where W is a good leaving group (e.g. succinimidyloxy (—OC₄H₄O₂) sulfosuccinimidyloxy (—OC₄H₃O₂—SO₃H), -1-oxybenzotriazolyl (—OC₆H₄N₃); or an aryloxy group or aryloxy substituted one or more times by electron withdrawing substituents such as nitro, fluoro, chloro, cyano, or trifluoromethyl, or combinations thereof, used to form activated aryl esters; or a carboxylic acid activated by a carbodiimide to form an anhydride or mixed anhydride —OCOAlk or —OCN(Alk₁)NH(Alk₂), where Alk₁ and Alk₂, which may be the same or different, are C₁-C₂₀ alkyl, C₁-C₂₀ perfluoroalkyl, or C₁-C₂₀ alkoxy; or cyclohexyl, 3-dimethylaminopropyl, or N-morpholinoethyl).
**Acyl azides can also rearrange to isocyanates.

The selection of a particular reactive group to attach the compound of the disclosure to the substance to be conjugated typically depends on the functional group present on the substance to be conjugated, and the type or length of covalent linkage desired. The types of functional groups typically present on the organic or inorganic substances include, but are not limited to, amines, amides, thiols, alcohols, phenols, aldehydes, ketones, phosphates, imidazoles, hydrazines, hydroxylamines, disubstituted amines, halides, epoxides, carboxylate esters, sulfonate esters, purines, pyrimidines, carboxylic acids, olefinic bonds, or a combination of these groups. A single type of reactive site may be available on the substance (typical for polysaccharides), or a variety of sites may be present (e.g. amines, thiols, alcohols, phenols), as is typical for proteins. A conjugated substance may be conjugated to more than one dye, which may be the same or different, or to a substance that is additionally modified by a hapten, such as biotin. Although some selectivity can be obtained by careful control of the reaction conditions, selectivity of labeling is best obtained by selection of an appropriate reactive dye.

Typically, the RM is selected to react with an amine, a thiol, an alcohol, an aldehyde or a ketone. Preferably RM is selected to react with an amine or a thiol functional group. In one embodiment, RM is an acrylamide, a reactive amine (including a cadaverine or ethylenediamine), an activated ester of a carboxylic acid (typically a succinimidyl ester of a carboxylic acid), an acyl azide, an acyl nitrile, an aldehyde, an alkyl halide, an anhydride, an aniline, an aryl halide, an azide, an aziridine, a boronate, a carboxylic acid, a diazoalkane, a haloacetamide, a halotriazine, a hydrazine (including hydrazides), an imido ester, an isocyanate, an isothiocyanate, a maleimide, a phosphoramidite, a reactive platinum complex, a sulfonyl halide, or a thiol group. By “reactive platinum complex” is particularly meant chemically reactive platinum complexes such as is described in U.S. Pat. Nos. 5,580,990; 5,714,327; and 5,985,566 (all hereby incorporated by reference).

Where the reactive moiety RM is a photoactivatable group, such as an azide, diazirinyl, azidoaryl, or psoralen derivative among others, the substituted dye becomes chemically reactive only after illumination with light of an appropriate wavelength. Where RM is an activated ester of a carboxylic acid, the reactive dye is particularly useful for preparing dye-conjugates of proteins, nucleotides, oligonucleotides, or haptens. Where RM is a maleimide or haloacetamide the reactive dye is particularly useful for conjugation to thiol-containing substances. Where RM is a hydrazide, the reactive dye is particularly useful for conjugation to periodate-oxidized carbohydrates and glycoproteins, and in addition is an aldehyde-fixable polar tracer for cell microinjection. Preferably, RM is a carboxylic acid, a succinimidyl ester of a carboxylic acid, a haloacetamide, a hydrazine, an isothiocyanate, a maleimide group, an aliphatic amine, a perfluorobenzamido, an azidoperfluorobenzamido group, or a psoralen. More preferably, RM is a succinimidyl ester of a carboxylic acid, a maleimide, an iodoacetamide, or a reactive platinum complex.

As used herein, by “water soluble moiety (WSM)” is meant a group that significantly increases the solubility of a cyanine compound of the disclosure. In one aspect, the compounds of the disclosure include at least one water soluble moiety (WSM). The typical WSM includes sulfonate, phosphonate, polyhydroxy, polyethylene glycol (PEG), ammonium, and pyridinium etc. WSM is used to increase the water solubility of the disclosed compounds.

As used herein, by “core pore glass moiety (CPG)” is meant a core pore glass solid support. In one aspect, CPG is a controlled pore glass as solid support for oligo nucleotide synthesis. Other equivalent solid supports may also be used.

### Phosphoramidite Moieties (CEP)

As used herein, by “phosphoramidite moiety (CEP)” is meant a hydroxy-reactive group used for oligo nucleotide synthesis. In one aspect, it typically has the following structure. Other equivalent phosphoramidite groups may also be used.

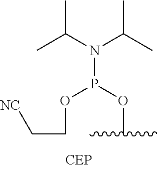 [SMILES: *OP(OCCC#N)N(C(C)C)C(C)C]

As used herein, by “conjugated substance” or a “conjugate” is an adduct of a cyanine of the disclosure bonded to a biological substance. Preparation of a conjugate of a dye of the disclosure confers the advantageous properties of the dye onto the resulting dye-conjugate. These advantageous properties make the resulting dye-conjugates useful for a wide variety of applications.

Preparation of the desired dye-conjugate begins with the selection of an appropriate reactive compound of the disclosure for the preparation of the desired dye-conjugate. Particularly useful dye-conjugates include, among others, conjugates of peptides, nucleotides, antigens, steroids, vitamins, drugs, haptens, metabolites, toxins, environmental pollutants, amino acids, proteins, nucleic acids, carbohydrates, lipids, ion-complexing moieties, or glass, plastic or other non-biological substances. Alternatively, the conjugated substance is a cell, cellular system, cellular fragment, or subcellular particle, e.g. inter alia, a virus particle, bacterial particle, virus component, biological cell (such as animal cell, plant cell, bacteria, yeast, or protist), or cellular component. Reactive dyes typically label functional groups at the cell surface, in cell membranes, organelles, or cytoplasm.

Typically the conjugated substance is an amino acid, peptide, protein, tyramine, polysaccharide, ion-complexing moiety, nucleoside, nucleotide, oligonucleotide, nucleic acid, hapten, psoralen, drug, hormone, lipid, lipid assembly, polymer, polymeric microparticle, biological cell or virus. More typically, the conjugated substance is a peptide, a protein, a nucleotide, an oligonucleotide, or a nucleic acid.

In another embodiment, the conjugated substance is a biological polymer such as a peptide, protein, oligonucleotide, or nucleic acid polymer that is also labeled with at least a second fluorescent dye (optionally an additional dye of the present disclosure), to form an energy-transfer pair. In some aspects, the labeled conjugate functions as an enzyme substrate, and enzymatic hydrolysis of the substrate disrupts energy transfer between the first and second dyes. Alternatively, the conjugated substance itself is a fluorescent dye (such as green fluorescent proteins and Phycobiliproteins).

In another embodiment, the conjugated substance is an amino acid (including those that are protected or are substituted by phosphates, carbohydrates, or C₁ to Cas carboxylic acids), or is a polymer of amino acids such as a peptide or protein. Preferred conjugates of peptides contain at least five amino acids, more preferably 5 to 36 amino acids. Preferred peptides include, but are not limited to, neuropeptides, cytokines, toxins, protease substrates, and protein kinase substrates. Preferred protein conjugates include enzymes, antibodies, lectins, glycoproteins, histones, albumins, lipoproteins, avidin, streptavidin, protein A, protein G, phycobiliproteins and other fluorescent proteins, hormones, toxins, chemokines and growth factors. In one preferred aspect, the conjugated protein is a phycobiliprotein, such as allophycocyanin, phycocyanin, phycoerythrin and allophycocyanin B (for example, see U.S. Pat. No. 5,714,386 to Roederer (1998); hereby incorporated by reference). Particularly preferred are conjugates of R-phycoerythrin and of allophycocyanin with selected dyes of the disclosure that serve as excited-state energy acceptors or donors. In these conjugates, excited state energy transfer results in long wavelength fluorescence emission when excited at relatively short wavelengths.

In yet another embodiment, the conjugated substance is a nucleic acid base, nucleoside, nucleotide or a nucleic acid polymer, including those that are modified to possess an additional linker or spacer for attachment of the compounds of the disclosure, such as an alkynyl linkage (U.S. Pat. No. 5,047,519), an aminoallyl linkage (U.S. Pat. No. 4,711,955), a heteroatom-substituted linker (U.S. Pat. No. 5,684,142), or other linkage (all hereby incorporated by reference). In another embodiment, the conjugated substance is a nucleoside or nucleotide analog that links a purine or pyrimidine base to a phosphate or polyphosphate moiety through a noncyclic spacer. In another embodiment, the dye is conjugated to the carbohydrate portion of a nucleotide or nucleoside, typically through a hydroxyl group but additionally through a thiol or amino group (U.S. Pat. Nos. 5,659,025, 5,668,268, 5,679,785). Typically, the conjugated nucleotide is a nucleoside triphosphate or a deoxynucleoside triphosphate or a dideoxynucleoside triphosphate. Incorporation of methylene moieties or nitrogen or sulfur heteroatoms into the phosphate or polyphosphate moiety is also useful. Nonpurine and nonpyrimidine bases such as 7-deazapurines (U.S. Pat. No. 6,150,510, incorporated by reference) and nucleic acids containing such bases can also be coupled to dyes of the disclosure. Nucleic acid adducts prepared by reaction of depurinated nucleic acids with amine, hydrazide or hydroxylamine derivatives provide an additional means of labeling and detecting nucleic acids, e.g. “A method for detecting abasic sites in living cells: age-dependent changes in base excision repair.” Atamna H, Cheung I, Ames B N. Proc Natl Acad Sci U.S. Pat. 97, 686-691 (2000).

Preferred nucleic acid polymer conjugates are labeled, single- or multi-stranded, natural or synthetic DNA or RNA, DNA or RNA oligonucleotides, or DNA/RNA hybrids, or incorporate an unusual linker such as morpholine derivatized phosphates, or peptide nucleic acids such as N-(2-aminoethyl)glycine units. When the nucleic acid is a synthetic oligonucleotide, it typically contains fewer than 50 nucleotides, more typically fewer than 25 nucleotides. Conjugates of peptide nucleic acids (PNA) (Nielsen et al U.S. Pat. No. 5,539,082) may be preferred for some applications because of their generally faster hybridization rates.

In another embodiment, the conjugated oligonucleotides of the disclosure are aptamers for a particular target molecule, such as a metabolite, dye, hapten, or protein. That is, the oligonucleotides have been selected to bind preferentially to the target molecule. Methods of preparing and screening aptamers for a given target molecule have been previously described and are known in the art (for example, U.S. Pat. No. 5,567,588 to Gold (1996); hereby incorporated by reference).

In one embodiment, conjugates of biological polymers such as peptides, proteins, oligonucleotides, nucleic acid polymers are also labeled with at least a second fluorescent dye, which is optionally an additional dye of the present disclosure, to form an energy-transfer pair. In some aspects of the disclosure, the labeled conjugate functions as an enzyme substrate, and enzymatic hydrolysis disrupts the energy transfer. In another embodiment of the disclosure, the energy-transfer pair that incorporates a dye of the disclosure is conjugated to an oligonucleotide that displays efficient fluorescence quenching in its hairpin conformation [the so-called “molecular beacons” of Tyagi et al., NATURE BIOTECHNOLOGY 16, 49 (1998)] or fluorescence energy transfer.

### Selected Embodiments

Selected examples of the compounds of this disclosure are provided in Table 2.

TABLE 2
Selected embodiment of the compounds of the disclosure. It is understood that
any of the structures shown may be present in a salt form thereof.

| Cpd. | |
|---|---|
| No. | Chemical Structure |
| 100 | 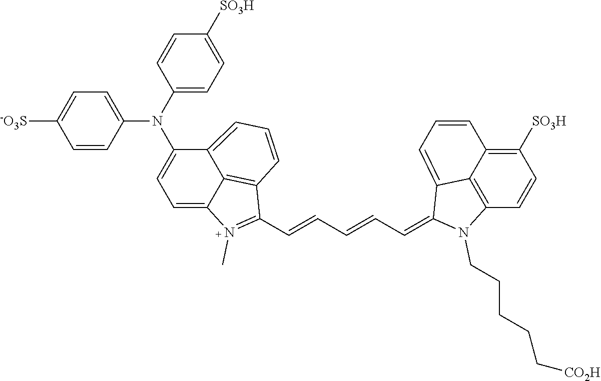 |
| 101 | 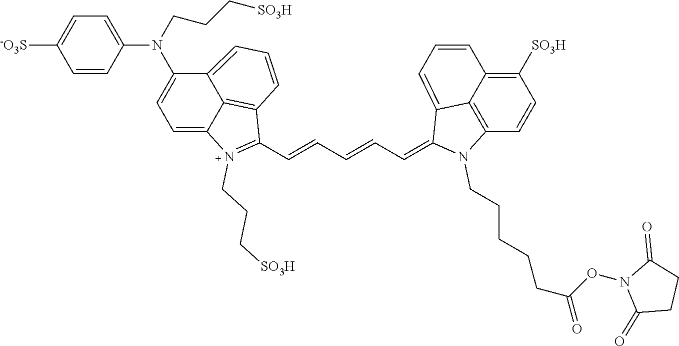 |
| 102 | 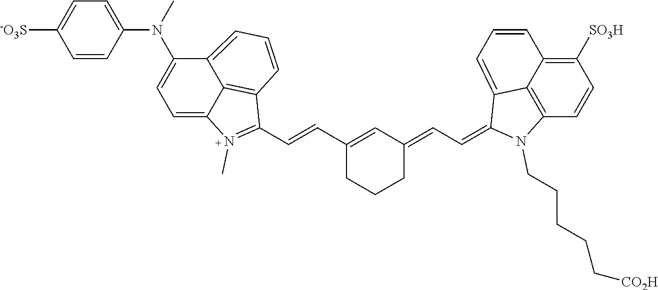 |
| 103 | 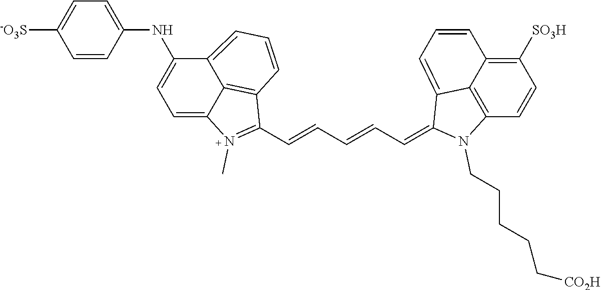 |
| 104 | 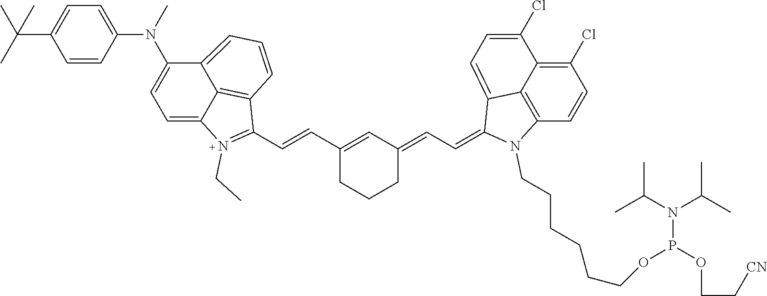 |
| 105 | 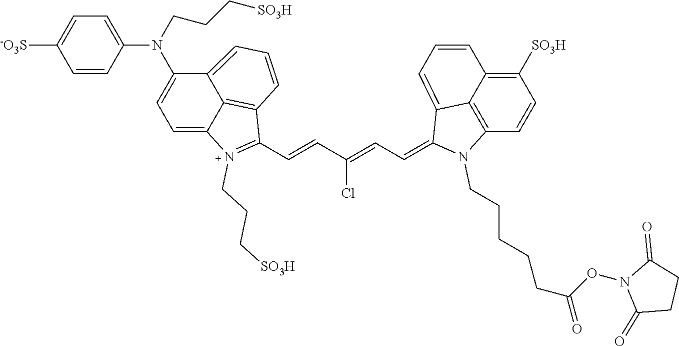 |
| 106 | 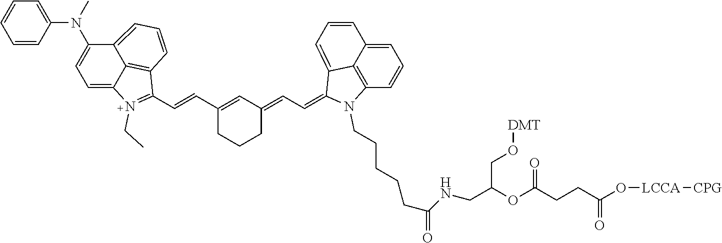 |
| 107 | 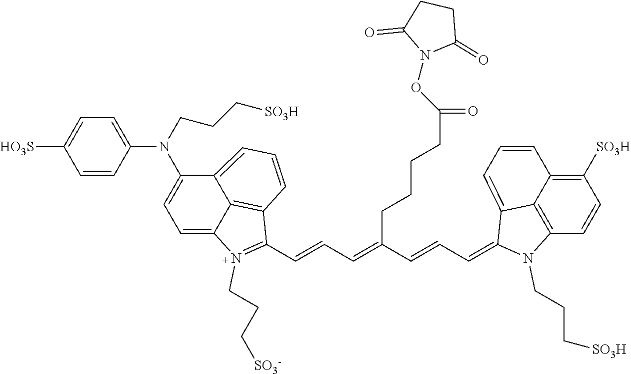 |
| 108 | 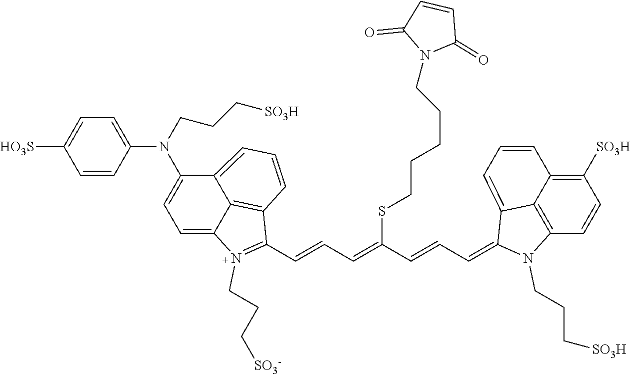 |
| 109 | 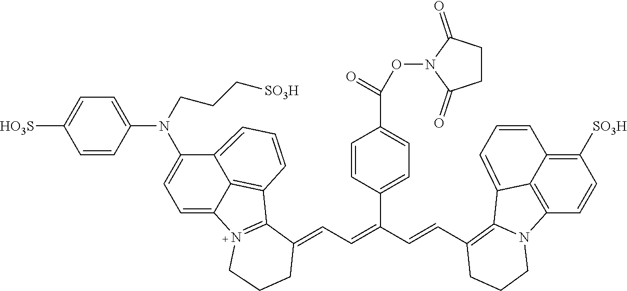 |
| 110 | 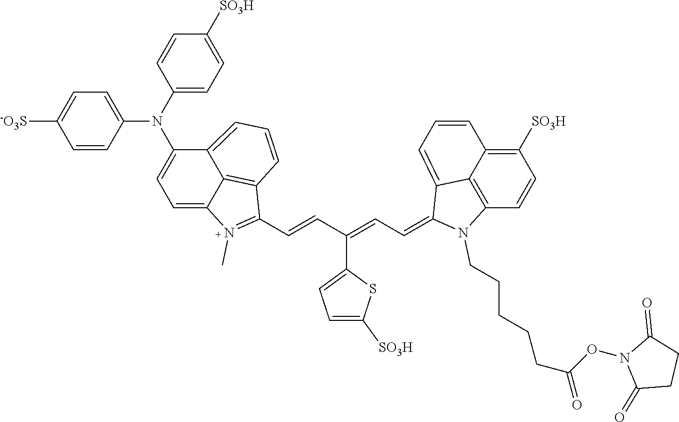 |
| 111 | 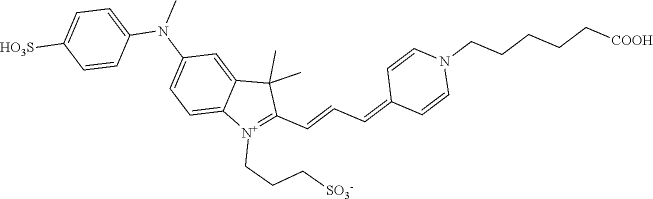 |
| 112 | 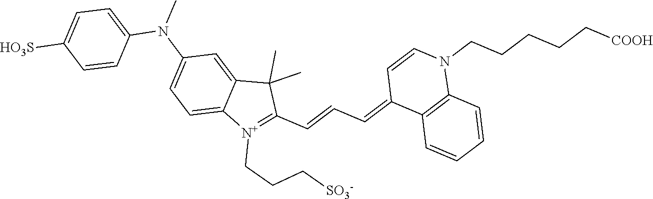 |
| 113 | 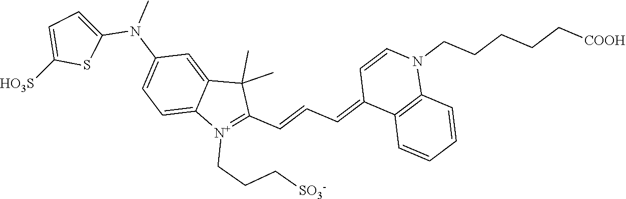 |
| 114 | 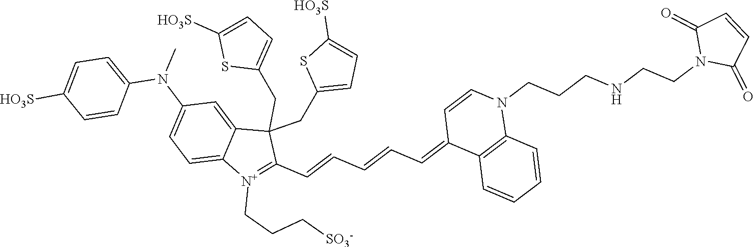 |
| 115 | 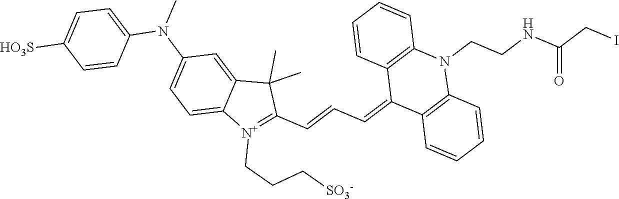 |
| 116 | 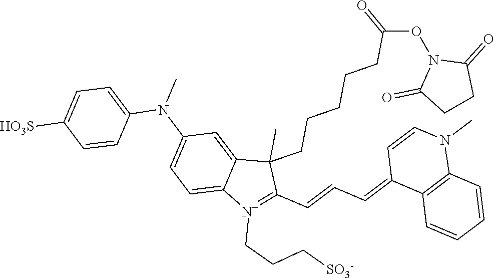 |
| 117 | 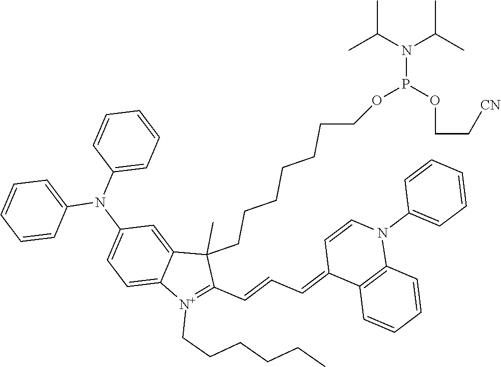 [SMILES: CCCCCC[N+]1=C(/C=C/C=C2\C=CN(c3ccccc3)c3ccccc32)C(C)(CCCCCCCOP(OCCC#N)N(C(C)C)C(C)C)c2cc(N(c3ccccc3)c3ccccc3)ccc21] |
| 118 | 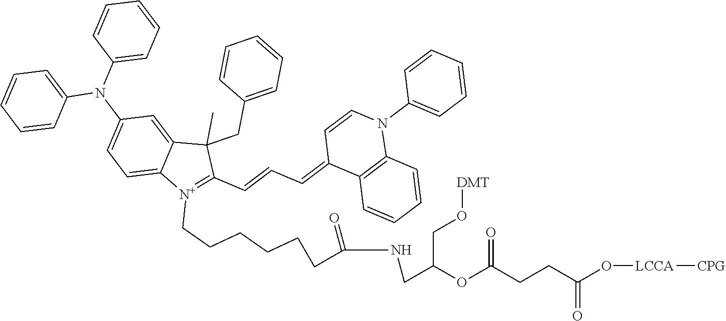 |
| 119 | 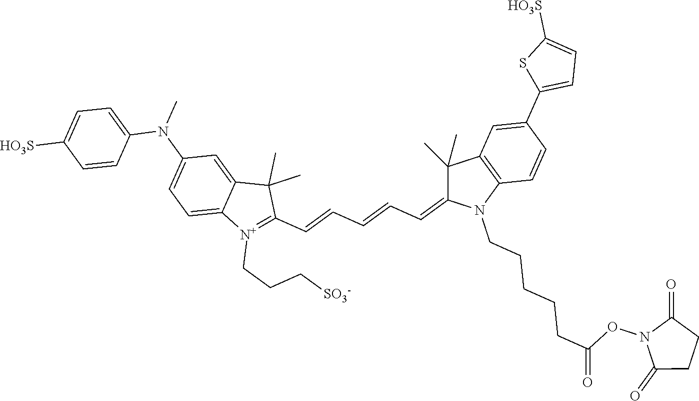 |
| 120 | 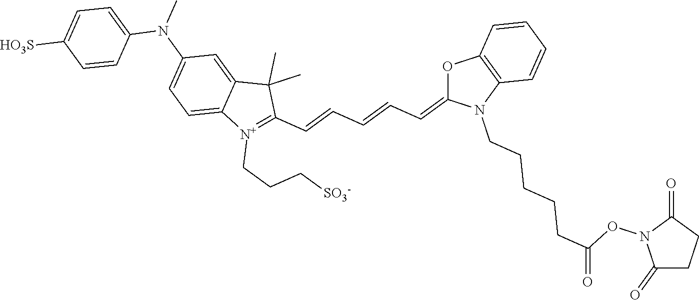 |
| 121 | 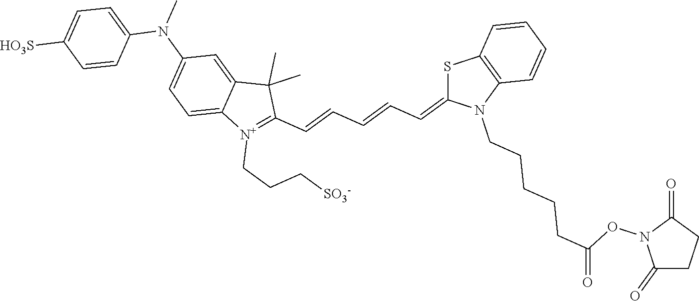 |
| 122 | 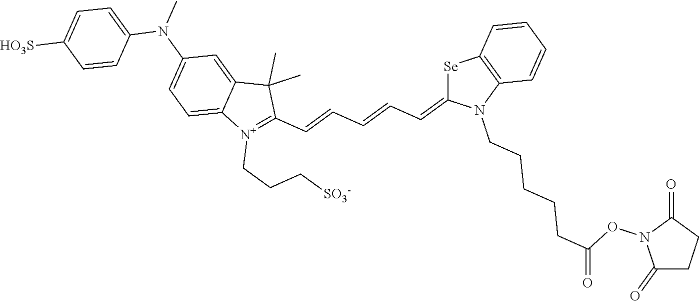 |
| 123 | 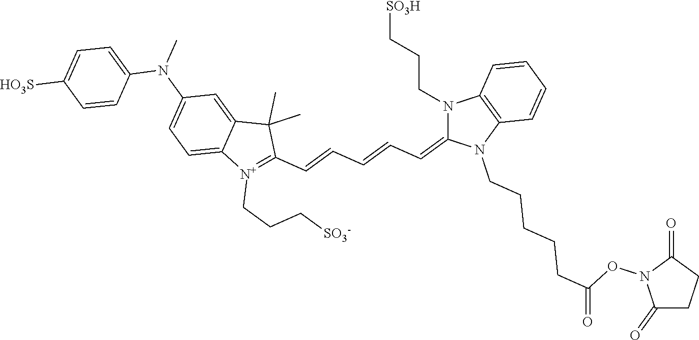 |
| 124 | 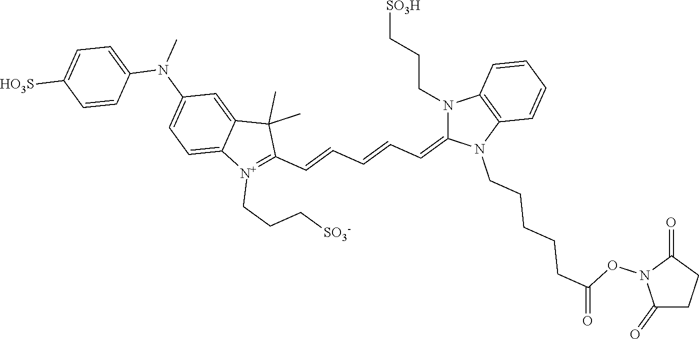 |
| 125 | 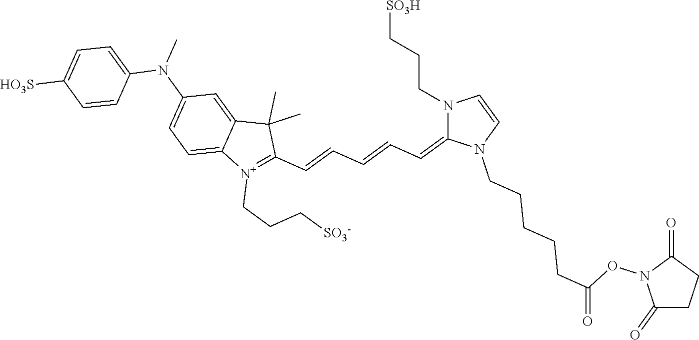 |
| 126 | 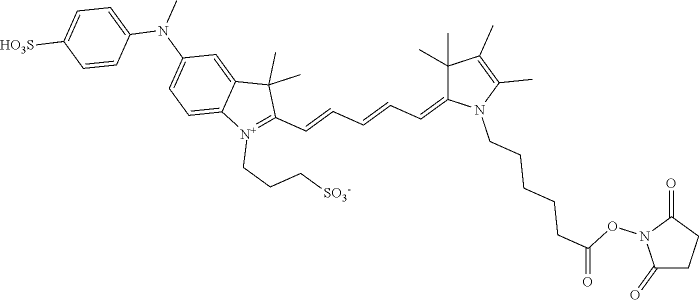 |
| 127 | 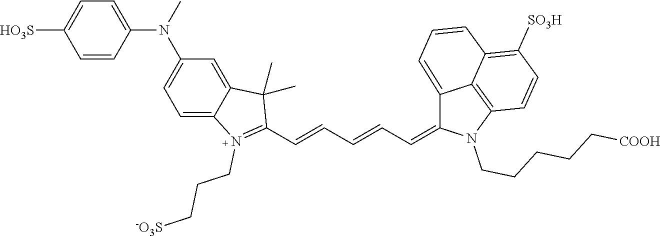 |
| 128 | 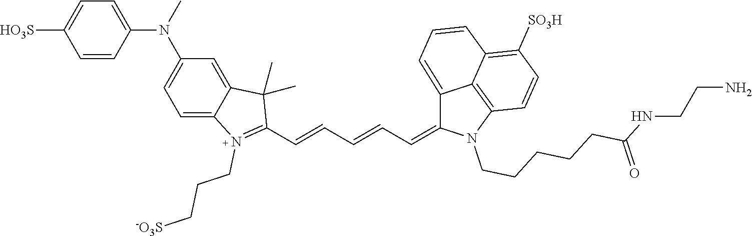 |
| 129 | 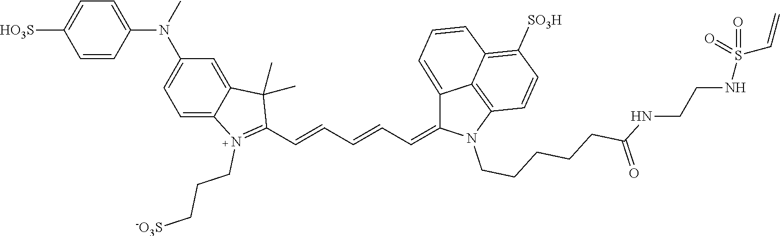 |
| 130 | 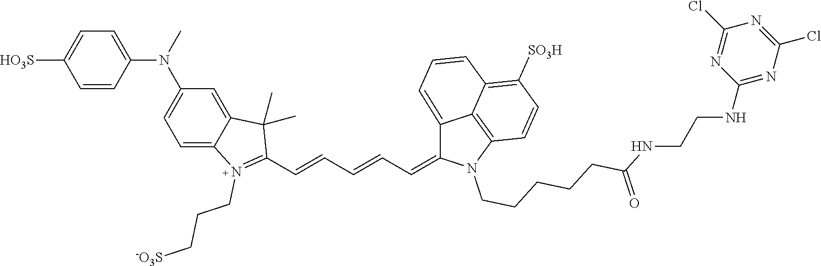 |
| 131 | 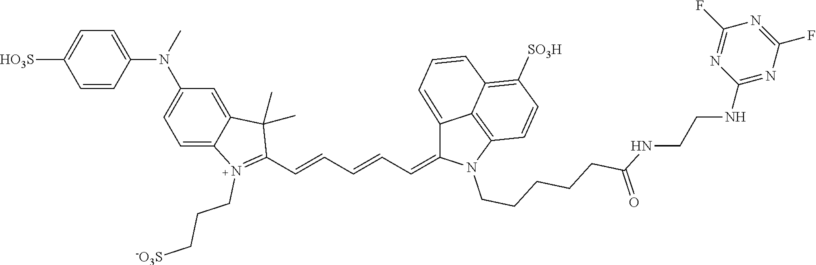 |
| 132 | 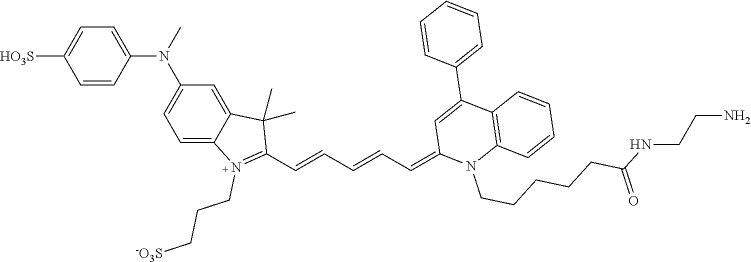 |
| 133 | 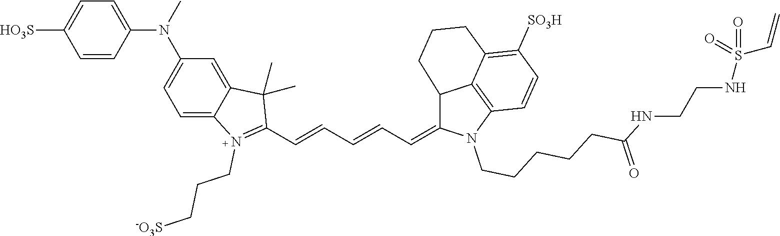 |
| 134 | 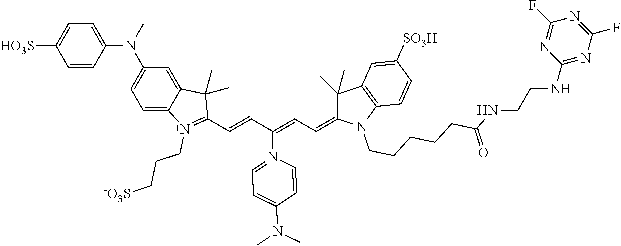 |
| 135 | 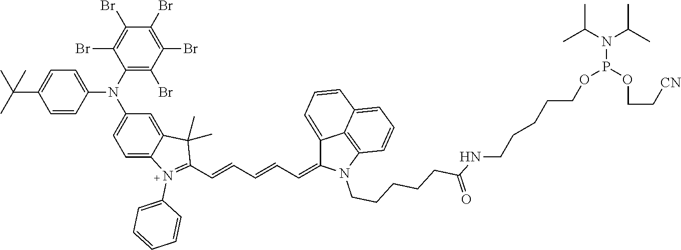 |
| 136 | 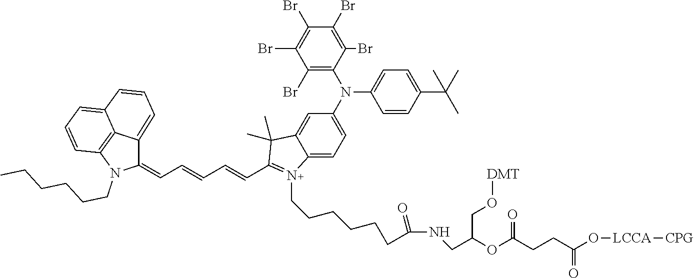 |
| 137 | 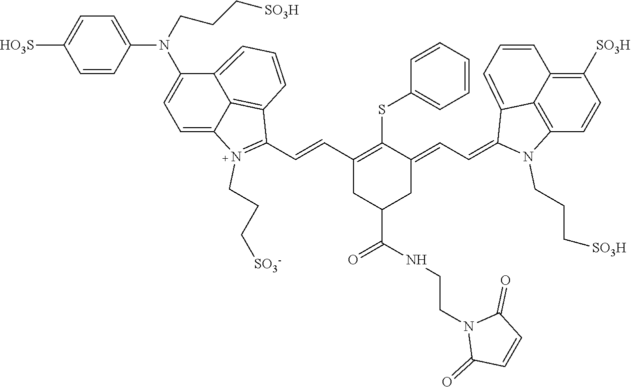 |

The preparation of dye conjugates using chemically reactive dyes is well documented (see HANDBOOK OF FLUORESCENT PROBES AND RESEARCH CHEMICALS, Chapters 1-5, thermofisher.com/us/en/home/references/molecular-probes-the-handbook; Brinkley, BIOCONJUGATE CHEM., 3, 2 (1992). Conjugates typically result from mixing appropriate reactive dyes and the substance to be conjugated in a suitable solvent, i.e. a solvent in which both components are soluble. The majority of the dyes of the disclosure are readily soluble in aqueous solutions, facilitating conjugation reactions with most biological materials. For those reactive dyes that are photoactivated, conjugation requires illumination of the reaction mixture to activate the reactive dye.

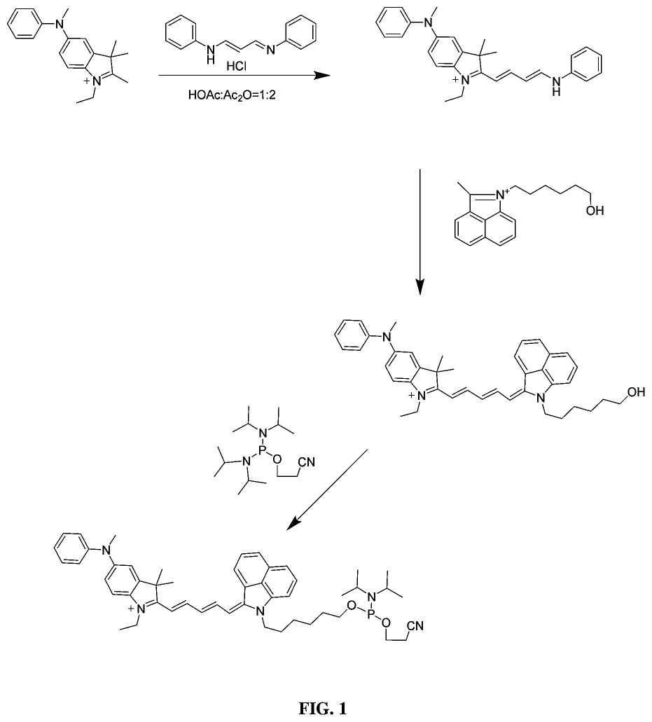

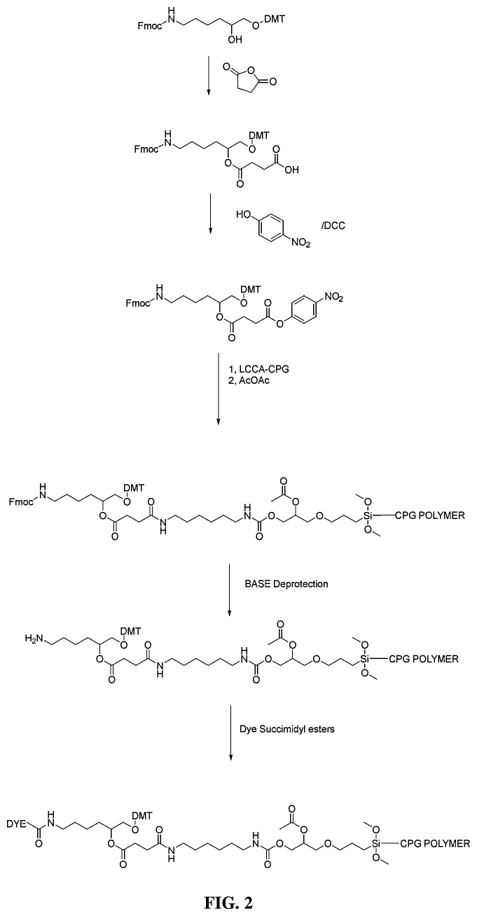

The preparation of cyanine dyes has been generally described in the literature (see Waggoner, A. et al., U.S. Pat. Nos. 5,627,027; 6,048,982; 6,207,464; Sasaki et al., U.S. Pat. No. 6,716,993; Pandey, R. K., et al., U.S. Pat. No. 9,821,062; NARAYANAN, N, et al., WO 2019/067180). Cyanine dyes may be prepared by the condensation of proper 2-methyl-substituted indolium, pyrrolium, oxazolium, thiazolium, imidazolium, pyridinium, or a quinolinium with a proper structure of X═CH—(CH═CH)ₙ—Y. X and Y can be O, Ph-NH, indoline, pyrrole, oxazoline, thiazoline, imidazoline, pyridine, or quinoline etc. These basic cyanine structures are optionally further substituted, during or after synthesis, to give the corresponding dye substituents as defined above (see Lee, L. G. et al., U.S. Pat. No. 6,348,596; Yano, T. et al., U.S. Pat. No. 8,958,165; Yoon, J., et al., U.S. Pat. No. 9,151,735; Chung, I. W. et al, U.S. Pat. No. 9,610,370). FIGS. 1 and 2 illustrate the typical synthetic routes. It is recognized that there are many possible variations that may yield an equivalent result.

Dyes which incorporate a reactive functional group, such as those described in Table 1 among others, may be prepared using using methods adapted from those documented in the literature. Particularly useful examples are amine-reactive dyes such as activated esters of carboxylic acids, which are typically synthesized by coupling a carboxylic acid to a relatively acidic leaving group. Other preferred amine-reactive groups include sulfonyl halides, which are prepared from sulfonic acids using a halogenating agent such as PCIs or POCl₃; halotriazines, which are prepared by the reaction of cyanuric halides with amines; isocyanates or isothiocyanates, which are prepared from amines and phosgene or thiophosgene; and phosphoramidites, which are prepared from alcohols or phenols respectively.

Dyes containing amines and hydrazides are particularly useful for conjugation to carboxylic acids, aldehydes and ketones. Most often these are synthesized by reaction of an activated ester of a carboxylic acid or a sulfonyl halide with a diamine, such as cadaverine, or with a hydrazine. Alternatively, aromatic amines are commonly synthesized by chemical reduction of a nitroaromatic compound. Amines and hydrazines are particularly useful precursors for synthesis of thiol-reactive haloacetamides or maleimides by standard methods.

Dye building blocks containing orthogonally protected amino acids such as FMOC-Lys (Dye)-OH, FMOC-Asp (Dye)-OH and FMOC-Glu (Dye) are readily used for the preparation of peptides that contain the compounds of the disclosure using the standard FMOC chemistry in an automated set up [E. Atherton, Sheppard, R. C. Solid Phase peptide synthesis: a practical approach. Oxford, England: IRL Press (1989); J. M. Stewart and J. D. Young, Solid phase peptide synthesis, 2nd ed, Rockford: Pierce Chemical Company (1984).J. Alternatively dye building blocks containing amino acids such as BOC-Lys (Dye)-OH, BOC-Asp (Dye)-OH and BOC-Glu (Dye) are readily used for the preparation of peptides that contain the compounds of the disclosure using the standard BOC chemistry or using liquid phase chemistry [N. Benoiton, Chemistry of Peptide Sythesis (2005); G. Jung, Combinatorial peptide and nonpeptide libraries, A Handbook, VCH: New York (1996)]. It is understood that quencher and/or fluorophore dyes can be installed (e.g., covalently connected or conjugated) at a variety of positions in a target peptide sequence, e.g., at the N-terminal, C-terminal, and/or connected to a sidechain of an amino acid residue. The installation can be achieved during peptide synthesis or via conjugation after synthesis, e.g., via a lysine reactive chemistry, a cysteine reactive chemistry, or the like.

Nucleosides and nucleotides labeled with dyes of the disclosure may be particularly useful for some applications of nucleic acid labeling. The use of dye phosphoramidites for labeling nucleotides and nucleosides have been previously described [U.S. Pat. Nos. 7,019,129 and 7,019,312 to Cook et al. (2006); U.S. Pat. No. 5,986,086 to Bruch et al. (1999); U.S. Pat. No. 5,808,044 to Brush et al. (1998); U.S. Pat. No. 5,556,959 to Brush et al. (1996) all hereby incorporated by reference].

Multiple dyes might be conjugated to a single large polymer such as an antibody, a protein, a carbohydrate, a lipid, a nucleic acid or a latex bead using standard bioconjugation techniques (see HANDBOOK OF FLUORESCENT PROBES AND RESEARCH CHEMICALS, Chapters 1-5, thermofisher.com/us/en/home/references/molecular-probes-the-handbook;

Brinkley, BIOCONJUGATE CHEM., 1992, 3, 2). Conjugates typically result from mixing appropriate reactive dyes and the substance to be conjugated in a suitable solvent in which both are soluble. The majority of the dyes of the disclosure are readily soluble in aqueous solutions, facilitating conjugation reactions with most biological materials. For those reactive dyes that are photoactivated, conjugation requires illumination of the reaction mixture to activate the reactive dye.

Examples of some synthetic strategies for selected dyes of the disclosure, as well as their characterization, synthetic precursors, conjugates and method of use are provided in the examples below. Further modifications and permutations will be obvious to one skilled in the art.

The compounds of the disclosure are useful as components in a variety of biological applications. For example, the dyes of the disclosure may be used as colorimetric labels for a conjugated substance. The compounds of the disclosure typically exhibit large extinction coefficients, and thereby permit the detection of the quenching compound-conjugated substance by virtue of visible light absorption of the conjugated compound.

The compounds of the disclosure are typically able to accept energy transfer from a wide variety of fluorophores, in this application, the compounds of the disclosure may be referred to as quenching compounds. Energy transfer may occur provided that the quenching compound and the fluorophore are in sufficiently close proximity for quenching to occur, and that at least some spectral overlap occurs between the emission wavelengths of the fluorophore and the absorption band of the quenching compound. This overlap may occur with emission of the donor occurring at a lower or even higher wavelength emission maximum than the maximal absorbance wavelength of the quenching compound, provided that sufficient spectral overlap exists. Energy transfer may also occur through transfer of emission of the donor to higher electronic states of the acceptor, such as from tryptophan residues of proteins to the weaker absorption bands between 300 and 350 nm typical of the dyes in the ultraviolet region. Preferably, the quenching compound of the disclosure is only dimly fluorescent, or substantially nonfluorescent, so that energy transfer results in little or no fluorescence emission. In one aspect of the disclosure, the quenching compound of the disclosure has a fluorescence quantum yield of less than about 0.1. In another aspect of the disclosure, the quenching compound has a fluorescence quantum yield of less than about 0.05. In yet another aspect of the disclosure, the quenching compound has a fluorescence quantum yield of less than about 0.01.

Typically, such fluorescence quenching occurs through Fluorescence Resonance Energy Transfer (FRET) between a donor and a quenching compound of the disclosure acting as an energy acceptor. The degree of FRET exhibited by a donor-acceptor pair can be represented by the equations [1] to [3]:

The efficiency (E %) and rate (kr) of FRET are respectively expressed as follows:

$$E \% = {k}_{T} / ( {τ}_{D}^{- 1} + {k}_{T} ) \qquad [ 1 ]$$

$${k}_{T} = {R}_{o}^{6} {γ}^{- 6} {τ}_{D}^{- 1} \qquad [ 2 ]$$

Where to is the decay time of the donor in the absence of acceptor; y is the donor-acceptor (D-A)distance; R, is the Förster distance where FRET has 50% efficiency, is typically in the range of 20-60 Å. R. is determined by the following equation:

$${R}_{o}^{6} = 8.79 \times {10}^{23} [ {k}^{2} {n}^{- 4} {Φ}_{D} J ( λ ) ] \qquad [ 3 ]$$

Where k² is dipole-dipole orientation factor (ranging from 0 to 4, k²=2/3 for randomly oriented donors and acceptors); n is refractive index [The refractive index is generally known from solvent composition or estimated for macromolecules such as proteins and nucleic acids. n is often assumed to be that of water (n=1.33) for aqueous solutions, or to be that of small organic molecules (n=1.39) for organic solutions]. @p is the fluorescence quantum yield of donor in the absence of acceptor. J (2) is FRET spectral overlap integral as determined by the following equation:

$$J ( λ ) = \int {F}_{D} ( λ ) {ε}_{A} ( λ ) {λ}^{4} d ( λ ) \qquad [ 4 ]$$

Where F_{D} (λ) is the corrected fluorescence intensity of the donor in the wavelength range λ to λ+Δλ with the total intensity (area under the curve) normalized unity; &A is extinction coefficient of the acceptor at λ.

From the above equations, it is easily concluded that for the most efficient FRET, the donor-acceptor pair should satisfy the following criteria:
Distance between donor and acceptor: Donor and acceptor molecules must be in close proximity (typically 10-100 Å).
Spectral overlap: The absorption spectrum of the acceptor must overlap fluorescence emission spectrum of the donor.
Dipole orientation: Donor and acceptor transition dipole orientations must be approximately parallel.

It should be readily appreciated that the degree of energy transfer during FRET, and therefore quenching, is highly dependent upon the separation distance between the fluorophore and the quenching compound. In molecular systems, a change in fluorescence quenching typically correlates well with a change in the separation distance between the fluorophore molecules and the quenching compound molecules. Assays that detect such changes in fluorescence are therefore useful for the detection of a great many structural changes, such as changes in molecular conformation, assembly of structures, or degradation of structures.

Any fluorophore with sufficient spectral overlap with a quenching compound of the disclosure, as calculated above, is a suitable donor for FRET applications, other factors being equal. The greater the degree of overlap, the greater the overall quenching observed. While fluorescent dyes are preferred for energy transfer applications, any emission that generates light having sufficient spectral overlap with the quenching compounds of the disclosure is also useful, such as chemifluorescence, or phosphorescence, whether by FRET or by triplet state to singlet state transfer.

While FRET is the most common mechanism for quenching of fluorescence to occur, any combination of molecular orientation and spectral coincidence that results in quenching of fluorescence is a useful mechanism for quenching by the quenching compounds of the disclosure, as described herein. For example, efficient quenching can occur even in the absence of spectral overlap if the fluorophore and the quenching compound are sufficiently close together to form a ground-state complex (as described in Tyagi et al., NATURE BIOTECHNOLOGY 16,49 (1998)).

Typically, the donor-acceptor includes a quenching compound of the disclosure and a fluorophore that is a fluorescent aromatic or heteroaromatic compound that is a carbocyanine, a porphyrin, a BODIPY, an oxazine or a benzoxazine, a rhodamine (see Kemnitzer et al., U.S. Pat. No. 11,180,657), a carbazine (see Corey et al., U.S. Pat. Nos. 4,810,636; 9,651,490), a silyl rhodamine (see Luke et al., U.S. Pat Appl. 2017/0045501), a fluorescent protein, conjugated polymer (see Gaylord et al., U.S. Pat. Nos. 10,288,620; 10,302,648; 10,485,989) or a nanocrystal (Adam, E. W. et al., U.S. Pat. No. 6,649,138). The donor dye is optionally an organic molecule that is a fluorophore, or a fluorescent protein such as a phycobiliprotein, a conjugated polymer, or a nanocrystal. Preferably, the donor dye is a carbazine, a rhodamine, an oxazine, a fluorescent cyanine. As used herein, oxazines include resorufins, aminooxazinones, diaminooxazines, and their benzo-substituted analogs. Preferred chemifluorescent dyes include luminol, isoluminol, luciferin, acridinium ester, or a dioxetane.

A FRET probe may be developed with a cyanine of the disclosure as the dark receptor where the fluorophore donor is a fluorescent cyanine (see Waggoner, A. et al., U.S. Pat. Nos. 5,627,027; 6,048,982; 6,207,464; Pandey, R. K., et al., U.S. Pat. No. 9,821,062). In another embodiment, the synthetic dye is optionally a fluorescein, a rhodol (see Haugland, et al., U.S. Pat. No. 5,227,487), or a rhodamine (see Kemnitzer et al., U.S. Pat. No. 11,180,657). As used herein, fluorescein includes benzo- or dibenzofluoresceins, seminaphthofluoresceins, or naphthofluoresceins. Similarly, as used herein rhodol includes seminaphthorhodafluors (see Haugland, et al., U.S. Pat. No. 4,945,171).

The quenching compounds of the disclosure are useful in any application where energy transfer from a fluorescent donor to a non-fluorescent acceptor has previously been described, provided that some spectral overlap exists between the emission of the donor dye and the absorbance of the quenching compound of the disclosure. Typically, the quenching compounds are used in combination with a fluorophore in a method that detects a change in separation distance between the fluorophore and the quenching compound.

The donor fluorophores and quenching compounds used in the instant methods are useful in any medium in which they are sufficiently soluble. For example, selected embodiments of the instant quenching compounds that are substituted by highly non-polar substituents may be useful in organic solvents, or on or in non-polar matrices, such as polymeric microspheres. For biological applications, the quenching compounds of the disclosure and their conjugates are typically used in an aqueous, mostly aqueous or aqueous-miscible solution prepared according to methods generally known in the art.

Chemically reactive compounds of the disclosure may covalently attach to a corresponding functional group on a wide variety of materials, forming conjugates as described above. Photoreactive compounds of the disclosure can be used similarly to photolabel nucleic acids, or components of the outer membrane of biological cells, or as photo-fixable polar tracers for cells.

The quenching compounds of the disclosure are generally utilized by labeling a substance or sample of interest under conditions selected so that illumination of the sample with an appropriate wavelength of light results in a detectable optical response. In one embodiment, the quenching compounds of the disclosure are utilized as colorimetric labels, such that the detectable optical response is an absorption of illumination energy. In another embodiment the quenching compound accepts energy from a donor, such that the detectable optical response is quenching of the fluorescence of the donor.

In most applications of the instant compounds, the labeled substance is utilized in a homogenous solution assay, where specific spatial resolution is not required. In these embodiments of the disclosure the loss of, or restoration of, fluorescence in the sample is detected. In another embodiment, the quenching compound forms a covalent or non-covalent association or complex with an element of the sample where a fluorescent component is present or is subsequently added. In this embodiment, illumination of the sample reveals either a fluorescence response if quenching is not occurring or the degree of quenching may be observed and correlated with a characteristic of the sample. Such correlation typically occurs by comparison with a standard or a calibration curve. Typically, a stained sample is illuminated and observed in order to determine a specified characteristic of the sample by comparing the degree of quenching exhibited to a fluorescence standard of determined intensity. The fluorescence standard may be a fluorescent dye such as the fluorophore used to prepare the quenching compound-fluorophore labeled substance, a fluorescent particle (including fluorescent microspheres), a calibration curve prepared by assaying the doubly labeled substance with a known amount of enzyme or degradation activity, or any other standard that can be used to calibrate fluorescence signal intensity as well known in the art.

Typically, the compounds of the disclosure are used in applications that yield information as to the separation distance between one or more fluorophore donors and quenching compound acceptors. These applications typically include the steps of illuminating the sample under study; detecting the fluorescence response of the system; exposing the sample to an environmental condition sufficient to change the separation distance, or thought to be sufficient to change the separation distance, illuminating the molecular system again, detecting the fluorescence response of the molecular system again, and comparing the first detected fluorescence response to the second detected fluorescence response. The difference in the detected fluorescence before and after the exposure to the selected environmental condition can then be correlated with any change that has occurred in the separation distance between the fluorophores and the quenching compounds, and thereby correlated with an effect of the selected environmental condition.

In one embodiment, the compounds of the disclosure are used in an application that includes preparing a sample that contains a dye-conjugate having the formula

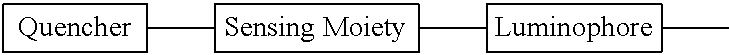

where the Quencher portion of the dye-conjugate is derived from a compound according to any one of Formulas I to XI; the Luminophore is a luminescent dye, including a fluorophore; and the Sensing Moiety is a substance selected to be capable of responding to a preselected environmental condition by changing the separation distance between the Quencher and the Luminophore (including fluorophore). Luminophore and fluorophore may be interchangeable.

The application or assay then includes detecting a first luminescence or fluorescence response of the sample; exposing the sample to an experimental environmental condition that is sufficient to change, or thought to be sufficient to change, the separation distance between the quencher and the luminophore/fluorophore; detecting a second luminescence or fluorescence response of said sample; determining a difference between the first and second luminescence or fluorescence responses; correlating the difference to a change in the separation distance between the quencher and the luminophore/fluorophore; and correlating the change in the separation distance between said quencher and said luminophore/fluorophore with the experimental environmental condition.

As discussed in greater detail below, the environmental conditions under which the instant method may be practiced may include the presence or absence of a particular enzyme, the presence or absence of a complementary specific binding pair member, a change in pH, or a change in sample temperature, among other conditions.

Typically, changes in fluorescence quenching may be detected by methods known in the art for standard fluorescence assays. The fluorescence of a sample, if present, is typically detected by illumination of the sample with a light source capable of producing light that is absorbed at or near the wavelength of maximum absorption of the donor dye, and fluorescence is detected at a wavelength longer than the excitation wavelength, typically near the emission maximum. Such illumination sources include, but are not limited to, hand-held ultraviolet lamps, mercury-arc lamps, xenon lamps, lasers and laser diodes. These illumination sources are optionally integrated into laser scanners, fluorescence microplate readers, standard or minifluorometers, or chromatographic detectors.

The optical response is optionally detected by visual inspection, or by use of instrumentation, including CCD cameras, video cameras, photographic film, laser-scanning devices, fluorometers, photodiodes, quantum counters, epifluorescence microscopes, scanning microscopes, flow cytometers, fluorescence microplate readers, among others. The optical response may be detected directly, or by virtue of a means of amplifying the signal, such as a photomultiplier tube. Where the sample is examined using a flow cytometer, examination of the sample optionally includes sorting portions of the sample according to their fluorescence response.

In the case of a sample in which the labeled substance is immobilized or partially immobilized on a solid or semi-solid support or in a matrix such as agar, sample fluorescence is typically detected using a transilluminator, an epi-illuminator, a laser scanner, a microscope or a similar apparatus that permits observation of the matrix.

Fluorescence occurring within a cell is typically detected using instrumentation that is capable of detecting fluorescent emission in single cells, such as a microscope or a flow cytometer (optionally further being followed by sorting of fluorescent cells). Alternatively, multiple cells may be suspended and fluorescence changes measured as for an assay done in true solution. As described above, the method of the instant disclosure is typically useful for detection of changes in separation distance between a fluorophore donor and a quenching compound acceptor. For example, the assay can provide for detection of an analyte by inducing a change (e.g., upon binding or other action of the analyte on the labelled substance) from a first separation distance to a second separation distance between the fluorophore donor and the quenching compound acceptor in the labelled substance or product thereof.

Any assay that relies upon the measurement of the proximity of fluorophores and quenching compounds in a system may be carried out using the method of the instant disclosure. The method of the instant disclosure is typically utilized to detect and/or quantify the convergence or divergence of the fluorophore donor and quenching compound acceptor. By convergence is meant a decrease in the average separation distance between the fluorophore and the quenching compound. By divergence is meant an increase in the average separation distance between the fluorophore and the quenching compound.

In one embodiment, the method of the instant disclosure is utilized to detect molecular or structural assembly (convergence). In another embodiment, the method of the disclosure is utilized to detect molecular or structural disassembly (divergence). In yet another embodiment, the method of the disclosure is utilized to detect a conformation change in a molecule, macromolecule or structure (optionally convergence or divergence). In yet another embodiment, the method of the instant disclosure incorporates aspects of the detection of assembly, disassembly, and/or conformation changes.

In one embodiment, the fluorescence of a fluorophore becomes quenched upon being placed in close proximity to a quenching compound of the disclosure (thereby decreasing the separation distance). The following systems, among others, can be analyzed using energy transfer pairs to detect and/or quantify structural assembly by measuring convergence of the donor and acceptor:
a) protein subunit assembly
b) enzyme-mediated protein assembly
c) molecular dimensions of proteins
d) membrane-protein interactions
e) protein-protein interactions
f) protein-protein-nucleic acid complex assembly
g) receptor/ligand interactions
h) immunoassays
i) nucleic acid hybridization
k) quantitative detection of specific DNA sequence amplification detection of DNA duplex winding
m) nucleic acid-protein interactions
n) nucleic acid-drug interactions
o) primer extension assays for mutation detection
p) reverse transcriptase assay
q) strand exchange in DNA recombination reactions
r) membrane fusion assays
s) transmembrane potential sensing
t) ligation assays

In particular, specific binding pair members labeled with a quenching compound are typically used as probes for the complementary member of that specific binding pair, by methods known in the art. The complementary member is typically labeled with a fluorescent label, and association of the two members of the specific binding pair results in fluorescence quenching. This assay is particularly useful in nucleic acid hybridization assays, evaluation of protein-nucleic acid interaction, and in selected standard immunoassays. In one embodiment, a loss of fluorescence indicates the association of an enzyme with an enzyme substrate, agonist or antagonist, such that the fluorophore on one is brought into close proximity to a quenching compound on the other. Selected preferred specific binding pair members are proteins that bind non-covalently to low molecular weight ligands (including biotin), oligonucleotides, and drug-haptens. Representative specific binding pairs are shown in Table 3.

TABLE 3
Representative Specific Binding Pairs

| antigen | Antibody |
|---|---|
| biotin | avidin, streptavidin, anti-biotin |
| folate | folate-binding protein |
| IgG* | protein A or protein G |
| drug | drug receptor |
| toxin | toxin receptor |
| carbohydrate | lectin or carbohydrate receptor |
| peptide | peptide receptor |
| protein | protein receptor |
| peptide nucleic acid | complementary strand |
| enzyme substrate | Enzyme |
| DNA (RNA) | CDNA (cRNA) |
| hormone | hormone receptor |
| ion | Chelator |

*IgG is an immunoglobulin; cDNA and cRNA are complementary strands used for hybridization

Alternatively, a monomer, labeled with a quenching compound, is incorporated into a polymer labeled with a fluorophore, resulting in quenching of fluorescence. In particular, a quenching compound-labeled nucleotide can be incorporated via the polymerase chain reaction into a double stranded DNA molecular that is labeled with a fluorophore.

In another embodiment of the method of the disclosure, the disassembly, cleavage or other degradation of a molecular structure is detected by observing the partial or complete restoration of fluorescence of a fluorophore donor. Typically, the initially quenched fluorescence of a fluorophore associated with the structure becomes dequenched upon being released from the constraint of being in close proximity to a quenching compound of the disclosure. The quenching compound is optionally associated with the same molecular structure as the fluorophore, or the donor and acceptor are associated with adjacent but distinct subunits of the structure. The following systems, among others, can be analyzed using energy transfer pairs to detect and/or quantify structural disassembly:
a). detection of protease activity using fluorogenic substrates (for example MMP protease assays)
b). detection of enzyme-mediated protein modification (e.g. cleavage of carbohydrates/fatty acids, phosphates, prosthetic groups)
c). immunoassays (via displacement/competitive assays)
d). detection of DNA duplex unwinding (e.g. helicase/topoisomerase/gyrase assays)
e). nucleic acid strand displacement
f). dsDNA melting
g). nuclease activity
h). lipid distribution and transport
i). TAQMAN assays

Structure disassembly is typically detected by observing the partial or complete restoration of fluorescence, as a conjugated substance is exposed to a degradation conditions of interest for a period of time sufficient for degradation to occur. A restoration of fluorescence indicates an increase in separation distance between the fluorophore and quenching compound, and therefore a degradation of the conjugated substance. If the detectable difference in fluorescence is detected as the degradation proceeds, the assay is a continuous assay. Since most enzymes show some selectivity among substrates, and as that selectivity can be demonstrated by determining the kinetic differences in their hydrolytic rates, rapid testing for the presence and activity of the target enzyme is provided by the enhancement of fluorescence of the labeled substrate following separation from the quenching compound.

In another embodiment of the disclosure, a single-stranded oligonucleotide signal primer is labeled with both a quenching compound and a fluorescent donor dye, and incorporates a restriction endonuclease recognition site located between the donor dye and the quenching compound. The single-stranded oligonucleotide is not cleavable by a restriction endonuclease enzyme, but upon binding to a complementary (target) nucleic acid, the resulting double stranded nucleic acid is cleaved by the enzyme and the decreased quenching is used to detect the presence of the complementary nucleic acid (see Nadeau et al., U.S. Pat. No. 5,846,726).

A single nucleotide polymorphism (SNP) can be detected through the use of sequence specific primers, by detection of melt temperatures of the double stranded nucleic acid. In this aspect, the complementary or substantially complementary strands are labeled with a quenching compound and a fluorophore donor, respectively, and dissociation of the two strands (melting) is detected by the restoration of fluorescence of the donor.

In yet another example of a divergence assay, the rupture of a vesicle containing a highly concentrated solution of fluorophores and quenching compounds is readily detected by the restoration of fluorescence after the vesicle contents have been diluted sufficiently to minimize quenching.

In this embodiment, the quenching compound and the fluorescent donor are present on the same or different substances, and a change in the three-dimensional structural conformation of one or more components of the assay results in either fluorescence quenching or restoration of fluorescence, typically by substantially decreasing or increasing the separation distance between the quenching compound and a fluorophore. The following systems, among others, can be analyzed using energy transfer pairs to detect and/or quantify conformation changes:
a). protein conformational changes
b). protein folding
c). structure and conformation of nucleic acids
d). drug delivery
e). antisense oligonucleotides
f). cell-cell fusion (e.g. via the diffusion apart of an initial donor-quenching compound pair)

By conformation change is meant, for example, a change in conformation for an oligonucleotide upon binding to a complementary nucleic acid strand. In one such assay, labeled oligonucleotides are substantially quenched when in solution, but upon binding to a complementary strand of nucleic acid become highly fluorescent [so-called “Molecular Beacons”, as described in European patent application EP 0 745 690, by Tyagi et al (1996)]. Another example detects the change in conformation when an oligonucleotide that has been labeled at its ends with a quenching compound and a fluorophore, respectively, loses its G-quartet conformation upon hybridization to a complementary sequence, resulting in decreased fluorescence quenching (see Pitner et al., U.S. Pat. No. 5,691,145). Alternatively, the binding of an enzyme substrate within the active site of a labeled enzyme may result in a change in tertiary or quaternary structure of the enzyme, with restoration or quenching of fluorescence.

When used in complex systems, especially in biological cells, the assays of the instant disclosure are optionally combined with the use of one or more additional detection reagents, such as an antibody, or a stain for another component of the system such as a nucleic acid stain, an organelle stain, a metal ion indicator, or a probe to assess viability of the cell. The additional detection reagent is optionally a fluorescent reagent exhibiting a color that contrasts with the donor dye present in the assay, or is a label that is detectable by other optical or non-optical properties.

One aspect of the instant disclosure is the formulation of kits that facilitate the practice of the methods of the disclosure, as described above. The kits of the disclosure comprises a quenching compound of the disclosure, or colorless quenching compound precursor of the disclosure, typically present conjugated to a nucleotide, oligonucleotide, nucleic acid polymer, peptide, or protein. Typically, the kits further comprise one or more buffering agents, typically present as an aqueous solution. The kits of the disclosure optionally further comprise additional detection reagents, a purification medium for purifying the resulting labeled substance, fluorescence standards, enzymes, enzyme inhibitors, organic solvent, or instructions for carrying out an assay of the disclosure.

In one embodiment, the kits comprise a quenching compound of the disclosure and a fluorescent donor. The quenching compound and fluorescent donor are optionally each attached to a conjugated substance, or present in solution as free compounds. Such a kit would be useful for the detection of cell-cell fusion, as fusion of a cell containing the quenching compound with a cell containing a fluorescent donor would result in quenching of fluorescence. Conjugation of either the quenching compound or the fluorescent donor or both to biomolecules, such as polysaccharides, would help retain the reagents in their respective cells until cell fusion occurred.

In another embodiment, the kits comprise a quenching compound and a fluorescent donor, each conjugated to a complementary member of a specific binding pair. In this aspect of the disclosure, binding of the two specific binding pair members results in quenching of fluorescence, and the kit is useful for the detection of competitive binding to one or the other specific binding pair members, or for the detection of an environmental condition or component that either facilitates or inhibits binding of the specific binding pair members.

In another embodiment, the kits comprise a conjugate of a quenching compound and a conjugate of a fluorescent donor, wherein the conjugates are selected such that the action of a particular enzyme results in covalent or noncovalent association of the two conjugates, resulting in quenching of fluorescence. Where the conjugated substances are nucleotides, oligonucleotides or nucleic acid polymers, the kit is useful for the detection of, for example, ligase, telomerase, helicase, topoisomerase, gyrase, DNA/RNA polymerase, or reverse transcriptase enzymes.

In another embodiment, the kits comprise a biomolecule that is covalently labeled by both a quenching compound of the disclosure and a fluorescent donor. In one aspect, the labeled biomolecule exhibits fluorescence until a specified environmental condition (such as the presence of a complementary specific binding pair) causes a conformation change in the biomolecule, resulting in the quenching of fluorescence.

Alternatively, the biomolecule is initially quenched, and a specified environmental condition (such as the presence of an appropriate enzyme or chemical compound) results in degradation of the biomolecule and restoration of fluorescence. Such a kit would be useful for the detection of complementary oligonucleotide sequences (as for MOLECULAR BEACONS), or for the detection of enzymes such as nuclease, lipase, protease, or cellulase.

### Enumerated Embodiments

Aspects of the disclosure are described in the following additional numbered embodiments.

Embodiment 1. A compound having Formula I:

WSM−{X—[CR₁═CR₂]ₙ—CR₃═Y]-RM Formula I

wherein:
n is an integer from 0 to 3;
X is an optionally substituted moiety selected from indolium, pyrrolium, oxazolium, a thiazolium, imidazolium, pyridinium, and quinolinium;
Y is an optionally substituted moiety selected from indoline, a pyrrole, oxazoline, a thiazoline, imidazoline, pyridine, and quinoline;
R₁ is H, halogen, or an optionally substituted moiety selected from alkyl, aryl, thiol, amino, and heterocycle;
R₂ is H, halogen, or an optionally substituted moiety selected from alkyl, aryl, thiol, amino, and heterocycle;
R₃ is H, halogen, or an optionally substituted moiety selected from alkyl, aryl, thiol, amino, and heterocycle;
RM is a chemically reactive moiety; and
WSM is a water-soluble moiety;
   provided that at least one of X and Y contain an optionally substituted arylamino or heteroarylamino group.

Embodiment 2. The compound of embodiment 1, wherein:
X is an optionally substituted indolium or quinolinium moiety;
Y is an optionally substituted indoline or quinoline moiety;
each of R₁, R₂, and R₃ are H;
RM is an activated ester; and
WSM is an optionally substituted sulfonate or phosphonate.

Embodiment 3. The compound of embodiment 1, wherein X is of Formula II:

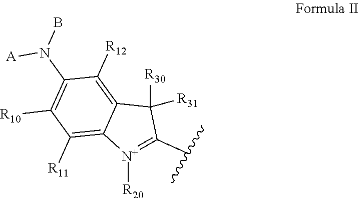

wherein:
A and B are each independently H or an optionally substituted alkyl, alkyl, aryl, aryl, heteroaryl, or heteroaryl;
R₁₀ to R₁₂ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boronic acid, aryl, and heteroaryl;
R₂₀ is an optionally substituted alkyl, aryl, or heteroaryl; and
R₃₀ and R₃₁ are each independently an optionally substituted moiety selected from alkyl, aryl, and heteroaryl;
   provided that at least one of A or B is aryl or heteroaryl.

Embodiment 4. The compound of embodiment 3, wherein:
A and B are independently H, or an optionally substituted alkyl or phenyl;
R₁₀ to R₁₂ are independently H, halogen, or an optionally substituted moiety selected from carboxy, alkyl, alkoxy, aryloxy, thiol, alkylthiol, arylthiol, aryl, and heteroaryl;
R₂₀ is an optionally substituted alkyl, aryl or heteroaryl; and
R₃₀ and R₃₁ are independently optionally substituted alkyl or aryl.

Embodiment 5. The compound of embodiment 4, wherein:
A and B are each independently H, or an optionally substituted sulfonated alkyl, or a sulfonated phenyl;
R₁₀ to R₁₂ are each independently H, halogen, or an optionally substituted moiety selected from carboxy, alkyl, alkoxy, aryloxy, aryl, and heteroaryl;
R₂₀ is a moiety selected from an optionally substituted alkyl, sulfonated alkyl and carboxylated alkyl; and
R₃₀ and R₃₁ are independently alkyl.

Embodiment 6. The compound of embodiment 3, wherein at least one of A and B is an optionally substituted sulfonated aryl or sulfonated heteroaryl.

Embodiment 7. The compound of embodiment 3, wherein at least one of A and B is sulfonated phenyl.

Embodiment 8. The compound of embodiment 3, wherein at least one of A and B is

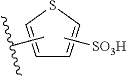

or a salt thereof.
Embodiment 9. The compound of embodiment 1, wherein X is of Formula III

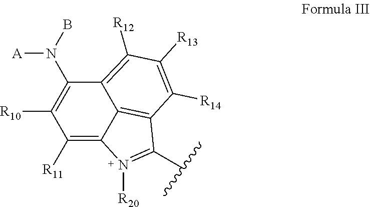

wherein:
A and B are each independently H, or an optionally substituted moiety selected from alkyl, aryl, and heteroaryl;
R₁₀ to R₁₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl; and
R₂₀ is an optionally substituted alkyl, aryl or heteroaryl;
   provided that at least one of A and B is an aryl or a heteroaryl.

Embodiment 10. The compound of embodiment 9, wherein:
A and B are independently H, or an optionally substituted moiety selected from alkyl and phenyl;
R₁₀ to R₁₄ are each independently H, halogen, or an optionally substituted moiety selected from carboxy, alkyl, alkoxy, aryloxy, thiol, alkylthiol, arylthiol, aryl, and heteroaryl; and
R₂₀ is an optionally substituted alkyl, aryl, or heteroaryl.

Embodiment 11. The compound of embodiment 9, wherein:
A and B are independently H, or an optionally substituted moiety selected from sulfonated alkyl and sulfonated phenyl; R₁₀ to R₁₄ are each independently H, halogen, or an optionally substituted moiety selected from carboxy, alkyl, alkoxy, aryloxy, aryl, and heteroaryl; R₂₀ is an optionally substituted alkyl, sulfonated alkyl or carboxylated alkyl.

Embodiment 12. A compound having Formula IV:

{X—[CR₁═CR₂]ₙ—CR₃═Y}—CPG Formula IV

wherein:
n is an integer from 0 to 3;
X is an optionally substituted moiety selected from indolium, pyrrolium, oxazolium, a thiazolium, imidazolium, pyridinium, and quinolinium;
Y is an optionally substituted moiety selected from indoline, pyrrole, oxazoline, thiazoline, imidazoline, pyridine, and quinoline;
R₁ to R₃ are each independently H, halogen, or an optionally substituted moiety selected from alkyl, aryl, thiol, amino, and heterocycle; and
CPG is a controlled pore glass as solid support for oligo nucleotide synthesis; provided that at least one of X and Y is an arylamino or a heteroarylamino group.

Embodiment 13. The compound of embodiment 12, wherein:
X is an optionally substituted indolium or quinolinium moiety;
Y is an optionally substituted indoline or quinoline moiety; and
R₁ to R₃ are each H.

Embodiment 14. The compound of embodiment 12, wherein X is of Formula II

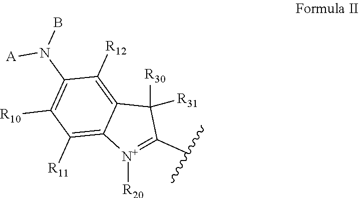

wherein:
A and B are each independently H, or an optionally substituted moiety selected from alkyl, aryl, and heteroaryl;
R₁₀ to R₁₂ are each independently H, halogen, or an optionally substituted carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ is an optionally substituted alkyl, aryl, or heteroaryl;
R₃₀ and R₃₁ are each independently optionally substituted alkyl, aryl, or heteroaryl provided that at least one of A and B is aryl or heteroaryl.

Embodiment 15. The compound of embodiment 14, wherein
A and B are each independently H, or an optionally substituted alkyl or phenyl;
R₁₀ to R₁₂ are each independently H, halogen, or an optionally substituted moiety selected from carboxy, alkyl, alkoxy, aryloxy, thiol, alkylthiol, arylthiol, aryl, and heteroaryl;
R₂₀ is an optionally substituted alkyl, aryl, or heteroaryl; and
R₃₀ and R₃₁ are each independently optionally substituted alkyl or aryl.

Embodiment 16. The compound of embodiment 12, wherein X is of Formula III

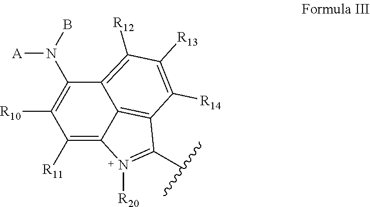

wherein:
A and B are each independently H, or an optionally substituted moiety selected from alkyl, aryl, and heteroaryl;
R₁₀ to R₁₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ is an optionally substituted alkyl, aryl, and heteroaryl;
   provided that at least one of A and B is aryl or heteroaryl.

Embodiment 17. The compound of embodiment 16, wherein:
A and B are each independently H, or an optionally substituted alkyl or phenyl;
R₁₀ to R₁₄ are each independently H, halogen, or an optionally substituted moiety selected from carboxy, alkyl, alkoxy, aryloxy, thiol, alkylthiol, arylthiol, aryl, and heteroaryl; and
R₂₀ is an optionally substituted alkyl, aryl or heteroaryl.

Embodiment 18. A compound having Formula V:

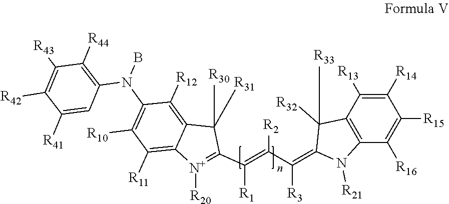

wherein:
B is H, or an optionally substituted alkyl, aryl, or heteroaryl;
n is an integer from 0 to 3;
R₁ to R₃ are each independently H, halogen, or an optionally substituted moiety selected from alkyl, aryl, thiol, amino, and heterocycle;
R₁₀ to R₁₆ and R₄₀ to R₃₃ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ and R₂₁ are each independently an optionally substituted alkyl, aryl or heteroaryl; and
R₃₀ to R₃₃ are each independently an optionally substituted alkyl, aryl, or heteroaryl;
   provided that at least there is a water soluble moiety and a reactive moiety.

Embodiment 19. The compound of embodiment 18, wherein:
B is H, or an optionally substituted alkyl or phenyl;
n is an integer from 1 to 3;
R₁ to R₃ are H;
R₁₀ to R₁₈ and R₄₀ to R₄₄ are each independently H, halogen, or an optionally substituted moiety selected from carboxy, alkyl, alkoxy, aryloxy, thiol, alkylthiol, arylthiol, sulfonate, aryl, and heteroaryl;
R₂₀ and R₂₁ are each independently an optionally substituted alkyl, carboxylated alkyl or sulfonated alkyl; and
R₃₀ to R₃₃ are an optionally substituted alkyl;
   provided that at least there are two sulfonates and a reactive moiety.

Embodiment 20. The compound of embodiment 18, wherein:
B is H, or an optionally substituted alkyl or sulfonated alkyl;
n is 1 or 2;
R₁ to R₃ are H;
R₁₀ to R₁₆ and R₄₀ to R₄₄ are each independently H, halogen, or an optionally substituted moiety selected from alkyl, alkoxy, sulfonate, aryl, and heteroaryl;
R₂₀ and R₂₁ are each independently optionally substituted moiety selected from alkyl, carboxylated alkyl, and sulfonated alkyl; and
R₃₀ to R₃₃ are each optionally substituted methyl;
   provided that at least there are two sulfonates and a reactive moiety.

Embodiment 21. A compound having Formula VI:

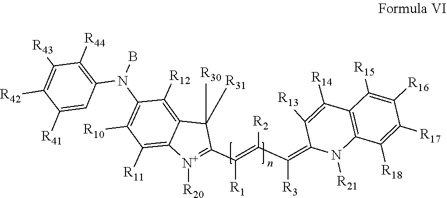

wherein:
B is H, or an optionally substituted alkyl, aryl or heteroaryl;
n is an integer from 0 to 3;
R₁ to R₃ are each independently H, halogen, or an optionally substituted moiety selected from alkyl, aryl, thiol, amino, and heterocycle;
R₁₀ to R₁₈ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ and R₂₁ are each independently optionally substituted alkyl, aryl, or heteroaryl;
R₃₀ and R₃₁ are each independently an optionally substituted alkyl, aryl or heteroaryl;
   provided that at least there is a reactive moiety and a water soluble moiety.

Embodiment 22. The compound of embodiment 21, wherein:
B is H, an optionally substituted alkyl or phenyl;
n is an integer from 1 to 3;
R₁ to R₃ are H;
R₁₀ to R₁₈ and R₄₀ to R₄₄ are each independently H, halogen, or an optionally substituted moiety selected from carboxy, alkyl, alkoxy, aryloxy, sulfonate, thiol, alkylthiol, arylthiol, aryl, and heteroaryl;
R₂₀ and R₂₁ are each independently optionally substituted alkyl, carboxylated alkyl or sulfonated alkyl; and
R₃₀ and R₃₁ are optionally substituted alkyl, provided that at least there are two sulfonates and a reactive moiety.

Embodiment 23. The compound of embodiment 21, wherein:
B is H, optionally substituted alkyl, or sulfonated alkyl;
n is 1 or 2;
R₁ to R₃ are H;
R₁₀ to R₁₈ and R₄₀ to R₄₄ are each independently H, halogen, or an optionally substituted moiety selected from alkyl, alkoxy, sulfonate, aryl, and heteroaryl;
R₂₀ and R₂₁ are each independently optionally substituted alkyl, carboxylated alkyl or sulfonated alkyl; and
R₃₀ and R₃₁ are each optionally substituted methyl;
   provided that at least there are two sulfonates and a reactive moiety.

Embodiment 24. A compound having Formula VII:

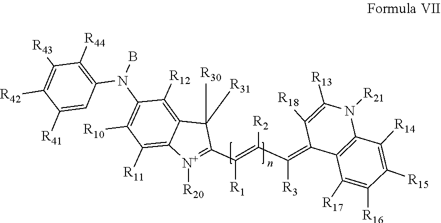

wherein:
B is H, or an optionally substituted moiety selected from alkyl, aryl and heteroaryl;
n is an integer from 0 to 3;
R₁ to R₃ are each independently H, halogen, or an optionally substituted moiety selected from alkyl, aryl, thiol, amino, and heterocycle;
R₁₀ to R₁₈ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ and R₂₁ are each independently optionally substituted alkyl, aryl, or heteroaryl; and
R₃₀ and R₃₁ are each independently optionally substituted alkyl, aryl, or heteroaryl;
   provided that at least there is a reactive moiety and a water soluble moiety.

Embodiment 25. The compound of embodiment 24, wherein:
B is H, optionally substituted alkyl or phenyl;
n is an integer from 1 to 3;
R₁ to R₃ are H;
R₁₀ to R₁₈ and R₄₀ to R₄₄ are each independently H, halogen, or an optionally substituted moiety selected from carboxy, alkyl, alkoxy, alkoxy, aryloxy, sulfonate, thiol, alkylthiol, arylthiol, aryl, and heteroaryl;
R₂₀ and R₂₁ are each independently an optionally substituted alkyl, a carboxylated alkyl, or sulfonated alkyl; and
R₃₀ and R₃₁ are each optionally substituted alkyl;
   provided that at least there are two sulfonates and a reactive moiety.

Embodiment 26. The compound of embodiment 24, wherein:
B is H, optionally substituted alkyl or sulfonated alkyl;
n is 1 or 2;
R₁ to R₃ are H;
R₁₀ to R₁₈ and R₄₀ to R₄₄ are each independently H, halogen, or an optionally substituted moiety selected from alkyl, alkoxy, sulfonate, aryl, and heteroaryl;
R₂₀ and R₂₁ are each independently optionally substituted alkyl, carboxylated alkyl, or sulfonated alkyl; and
R₃₀ and R₃₁ are each optionally substituted methyl;
   provided that at least there are 2 sulfonates and a reactive moiety.

Embodiment 27. A compound having Formula VIII:

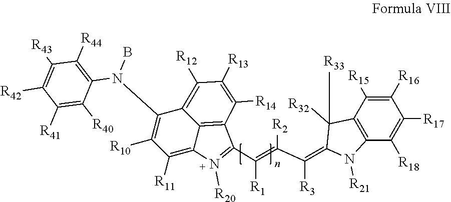

wherein:
B is H, or an optionally substituted alkyl, aryl, or heteroaryl;
n is an integer from 0 to 3;
R₁ to R₃ are each independently H, halogen, or an optionally substituted moiety selected from alkyl, aryl, thiol, amino, and heterocycle;
R₁₀ to R₁₈ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ and R₂₁ are each, independently an optionally substituted alkyl, aryl, or heteroaryl; and
R₃₂ and R₃₃ are each independently an optionally substituted alkyl, aryl, or heteroaryl;
   provided that at least there is a reactive moiety and a water soluble moiety.

Embodiment 28. The compound of embodiment 27, wherein:
B is H, or an optionally substituted alkyl or phenyl;
n is an integer from 1 to 3;
R₁ to R₃ are H;
R₁₀ to R₁₈ and R₄₀ to R₄₄ are each independently H, halogen, or an optionally substituted moiety selected from carboxy, alkyl, alkoxy, aryloxy, sulfonate, thiol, alkylthiol, arylthiol, aryl, and heteroaryl;
R₂₀ and R₂₁ are each independently optionally substituted alkyl, carboxylated alkyl or sulfonated alkyl; and
R₃₂ and R₃₃ are an optionally substituted alkyl;
   provided that at least there are two sulfonates and a reactive moiety.

Embodiment 29. The compound of embodiment 27, wherein:
B is H, or an optionally substituted alkyl or sulfonated alkyl;
n is 1 or 2;
R₁ to R₃ are H;
R₁₀ to R₁₈ and Rao to R₄₄ are each independently H, halogen, or an optionally substituted moiety selected from alkyl, alkoxy, sulfonate, aryl, and heteroaryl;
R₂₀ and R₂₁ are each independently an optionally substituted carboxylated alkyl or sulfonated alkyl; and
R₃₂ and R₃₃ are each an optionally substituted methyl;
   provided that at least there are two sulfonates and a reactive moiety.

Embodiment 30. A compound having Formula IX:

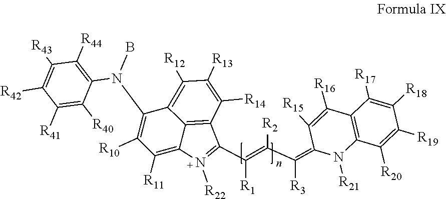

wherein:
B is H, optionally substituted alkyl, aryl, or heteroaryl;
n is an integer from 0 to 3;
R₁ to R₃ are each independently H, halogen, optionally substituted alkyl, aryl, thiol, amino, or heterocycle;
R₁₀ to R₂₀ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl; and
R₂₁ and R₂₂ are each independently optionally substituted alkyl, aryl, or heteroaryl;
   provided that at least there is a reactive moiety.

Embodiment 31. The compound of embodiment 30, wherein:
B is H, optionally substituted alkyl or phenyl;
n is an integer from 1 to 3;
R₁ to R₃ are H;
R₁₀ to R₂₀ and R₄₀ to R₄₄ are each independently H, halogen, or an optionally substituted moiety selected from carboxy, alkyl, alkoxy, aryloxy, aryloxy, sulfonate, thiol, alkylthiol, arylthiol, aryl, and heteroaryl; and
R₂₁ and R₂₂ are each independently optionally substituted alkyl, carboxylated alkyl, or sulfonated alkyl;
   provided that at least there is a reactive moiety.

Embodiment 32. The compound of embodiment 30, wherein:
B is H, optionally substituted alkyl or sulfonated alkyl;
n is 1 or 2;
R₁ to R₃ are H;
R₁₀ to R₂₀ and R₄₀ to R₄₄ are each independently H, halogen, or an optionally substituted alkyl, alkoxy, sulfonate, aryl, or heteroaryl; and
R₂₁ and R₂₂ are each independently optionally substituted carboxylated alkyl or sulfonated alkyl;
   provided that at least there are two sulfonates and a reactive moiety.

Embodiment 33. A compound having Formula X:

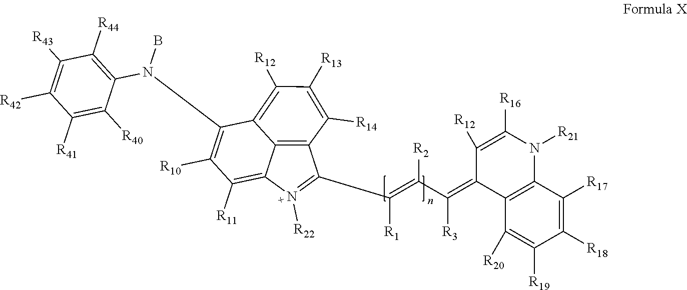

wherein:
B is H, optionally substituted alkyl, aryl, or heteroaryl;
n is an integer from 0 to 3;
R₁ to R₃ are each independently H, halogen, or an optionally substituted moiety selected from alkyl, aryl, thiol, amino, and heterocycle;
R₁₀ to R₂₀ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl; and
R₂₁ and R₂₂ are each independently optionally substituted alkyl, aryl, or heteroaryl;
   provided that at least there is a reactive moiety.

Embodiment 34. The compound of embodiment 33, wherein:
B is H, an optionally substituted alkyl or phenyl;
n is an integer from 1 to 3;
R₁ to R₃ are H;
R₁₀ to R₂₀ and R₄₀ to R₄₄ are each independently H, halogen, or an optionally substituted moiety selected from carboxy, alkyl, alkoxy, aryloxy, sulfonate, thiol, alkylthiol, arylthiol, aryl, and heteroaryl; and
R₂₁ and R₂₂ are each independently an optionally substituted alkyl, carboxylated alkyl, or sulfonated alkyl;
   provided that at least there are two sulfonates and a reactive moiety.

Embodiment 35. The compound of embodiment 33, wherein:
B is H, an optionally substituted alkyl or sulfonated alkyl;
n is 1 or 2;
R₁ to R₃ are H;
R₁₀ to R₂₀ and R₄₀ to R₄₄ are each independently H, halogen, or an optionally substituted moiety selected from alkyl, alkoxy, sulfonate, aryl, and heteroaryl; and
R₂₁ and R₂₂ are each independently carboxylated alkyl or sulfonated alkyl.

Embodiment 36. A compound having Formula XI:

{X—[CR₁═CR₂]ₙ—CR₃═Y}—CEP Formula XI

wherein:
n is an integer from 0 to 3;
X is an optionally substituted moiety selected from indolium, pyrrolium, oxazolium, thiazolium, imidazolium, pyridinium, and quinolinium;
Y is an optionally substituted moiety selected from indoline, pyrrole, oxazoline, thiazoline, imidazoline, pyridine, and quinoline;
R₁ to R₃ are each independently H, halogen, or an optionally substituted moiety selected from alkyl, aryl, thiol, amino and heterocycle; and
CEP is a phosphoramidite moiety;
   provided that at least one of X and Y contains an optionally substituted arylamino or heteroarylamino group.

Embodiment 37. The compound of embodiment 36, X is an optionally substituted moiety selected from indolium and quinolinium;
Y is an optionally substituted moiety selected from indoline and quinoline;
R₁ to R₃ are H.

Embodiment 38. The compound of embodiment 36, wherein X has Formula II

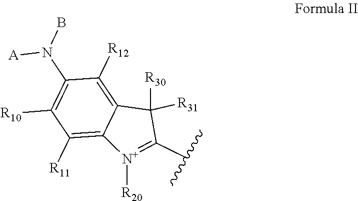

wherein:
A and B are each independently H, or an optionally substituted moiety selected from alkyl, aryl, and heteroaryl;
R₁₀ to R₁₂ are each independently H, halogen, or an optionally substituted carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ is an optionally substituted alkyl, aryl, or heteroaryl;
R₃₀ and R₃₁ are each independently optionally substituted alkyl, aryl, or heteroaryl provided that at least one of A and B is aryl or heteroaryl.

Embodiment 39. The compound of embodiment 38, wherein:
A and B are each independently H, or an optionally substituted alkyl or phenyl;
R₁₀ to R₁₂ are each independently H, halogen, or an optionally substituted moiety selected from carboxy, alkyl, alkoxy, aryloxy, thiol, alkylthiol, arylthiol, aryl, and heteroaryl;
R₂₀ is an optionally substituted alkyl, aryl, or heteroaryl; and
R₃₀ and R₃₁ are each independently optionally substituted alkyl or aryl.

Embodiment 40. The compound of embodiment 36, wherein X has Formula III

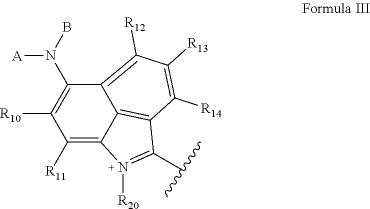

wherein:
A and B are each independently H, or an optionally substituted moiety selected from alkyl, aryl, and heteroaryl;
R₁₀ to R₁₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ is an optionally substituted alkyl, aryl, and heteroaryl;
   provided that at least one of A and B is aryl or heteroaryl.

Embodiment 41. The compound of embodiment 40, wherein:
A and B are each independently H, or an optionally substituted alkyl or phenyl;
R₁₀ to R₁₄ are each independently H, halogen, or an optionally substituted moiety selected from carboxy, alkyl, alkoxy, aryloxy, thiol, alkylthiol, arylthiol, aryl, and heteroaryl; and
R₂₀ is an optionally substituted alkyl, aryl or heteroaryl.

Embodiment 42. A dye-conjugate having Formula XII:

[Quencher]−[Sensing Moiety]−[Fluorophore] Formula XII

wherein:
Quencher is a quenching compound prepared from a compound of any one of embodiments 1-41;
Sensing Moiety is a molecule capable of specifically binding to, or being cleaved by, an analyte of interest; and
Fluorophore is a fluorescent dye;
   wherein the Sensing Moiety is bound or covalently linked to both the Quencher and the Fluorophore.

Embodiment 43. The dye-conjugate according to embodiment 42, wherein the Sensing Moiety comprises a peptide, a nucleotide, a protein, a nucleic acid or a carbohydrate.

Embodiment 44. The dye-conjugate according to embodiment 42, wherein the Fluorophore is a rhodamine dye, a cyanine dye, an oxazine dye, a BODIPY dye, a lanthanide complex dye, or a ruthenium complex dye.

Embodiment 45. The dye-conjugate according to claim 42, wherein the analyte of interest is a protease enzyme and the Sensing Moiety is a substrate for the enzyme.

Embodiment 46. The dye-conjugate according to claim 42, wherein the analyte of interest is a nucleotide capable of binding to the Sensing Moiety.

Embodiment 47. A method of detecting an analyte, comprising:
a) providing a sample that comprises a dye-conjugate having Formula XII

[Quencher]−[Sensing Moiety]−[Fluorophore] Formula XII

wherein:
Quencher is a quenching compound prepared from a compound of any one of claims 1-41;
Sensing Moiety is a molecule capable of binding to, or being cleaved by, an analyte of interest;
Fluorophore is a fluorescent dye;
   wherein:
the Sensing Moiety is configured to respond to a preselected environmental condition via changing a first separation distance between the Quencher and the Luminophore; and
the Sensing Moiety is bound or covalently linked to both the Quencher and the Fluorophore whereby FRET occurs between the Quencher and the Fluorophore at the first separation distance;
b) detecting a first luminescence response of said sample;
c) exposing the sample to an experimental environmental condition that is sufficient to change, or suspected to be sufficient to change, the first separation distance between the quencher and the Luminophore;
d) detecting a second luminescence response of said sample;
e) determining a difference between said first and second luminescence responses;
f) correlating said difference to a change in the separation distance between said Quencher and said Fluorophore; and
g) correlating said change in the separation distance between said Quencher and said Fluorophore with said experimental environmental condition.

Embodiment 48. An assay kit, comprising:
a) a dye-conjugate according to any one of embodiments 42-46; and
b) a biological buffer.

Embodiment 49. A compound selected from Table 2.

Embodiment 50. An assay kit comprising a compound selected from Table 2.

Embodiment 51. A compound selected from Table 4.

Embodiment 52. An assay kit comprising a compound selected from Table 4.

Embodiment 53. A method of labeling a peptide, oligopeptide, or protein, the method comprising:
contacting a peptide, oligopeptide, or protein comprising a nucleophilic amino acid residue with a compound of any one of embodiments 1 to 41 to produce a labeled peptide, oligopeptide, or protein.

Embodiment 54. The method of embodiment 53, further comprising assessing (e.g., measuring) HIV protease activity, wherein the labeled peptide, oligopeptide, or protein comprises an enzyme-cleavable site.

Embodiment 55. A method of labeling a nucleic acid oligomer or polymer, the method comprising:
contacting a nucleic acid oligomer or polymer comprising a nucleophilic nucleic acid residue with a compound of any one of embodiments 1 to 41 to produce a labeled nucleic acid oligomer or polymer.

Embodiment 56. The method of embodiment 55, further comprising assessing (e.g., measuring) reverse transcriptase activity.

Embodiment 57. The method of embodiment 55, further comprising assessing (e.g., measuring) nuclease activity.

Embodiment 58. The method of embodiment 55, further comprising assessing (e.g., measuring) ligase activity.

Embodiment 59. The method of embodiment 55, further comprising assessing (e.g., measuring) topoisomerase activity.

Embodiment 60. The method of embodiment 55, further comprising assessing (e.g., measuring) metalloproteinase activity.

The examples provided below are given so as to illustrate the practice of this disclosure. They are not intended to limit or define the scope of the disclosure.

In some embodiments, the compound which finds use in the compounds, conjugates and compositions of this disclosure is of Table 4.

TABLE 4
It is understood that any of the structures shown may be present in a salt form thereof.

| Cpd. | |
|---|---|
| No. | Chemical Structure |
| 3 | 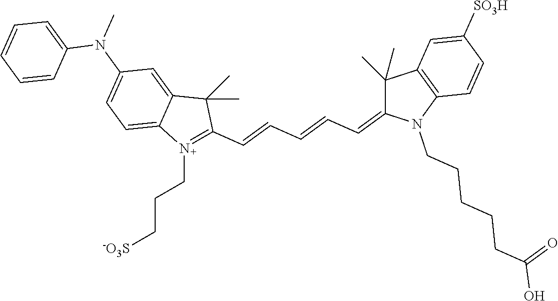 |
| 4 | 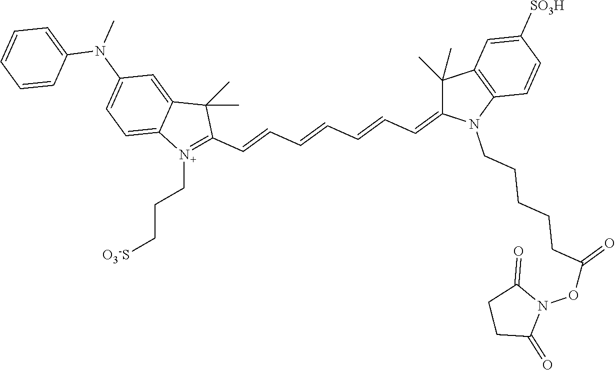 |
| 10 | 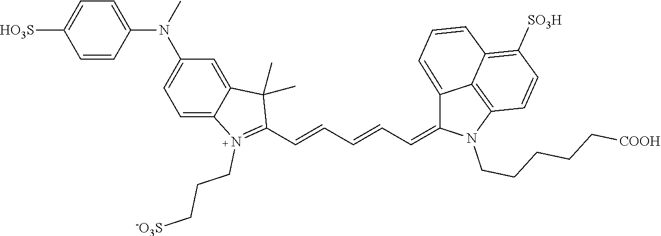 |
| 11 | 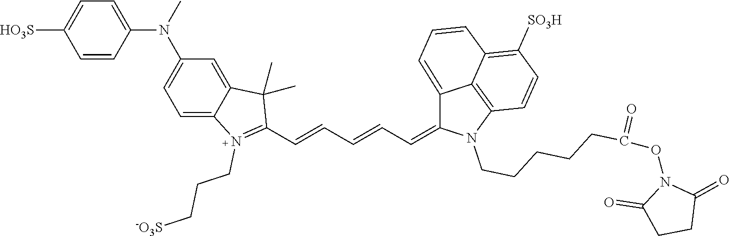 |
| 12 | 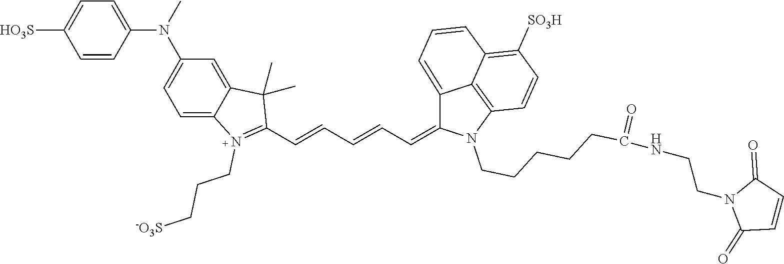 |
| 13 | 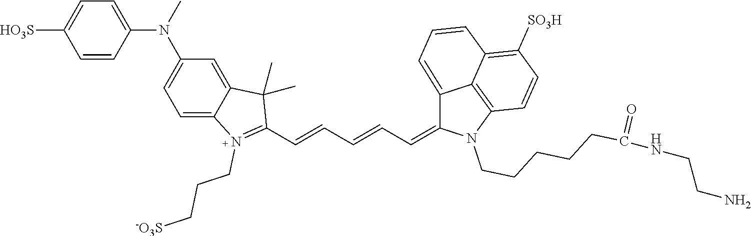 |
| 14 | 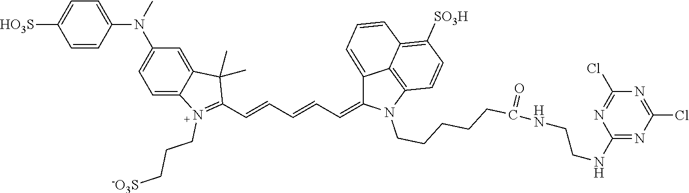 |
| 15 | 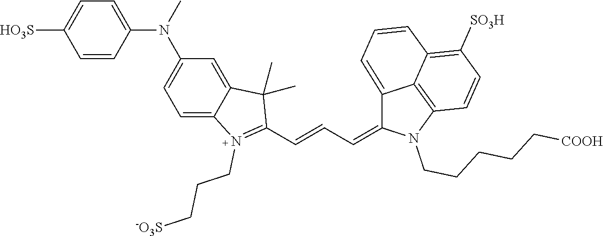 |
| 21 | 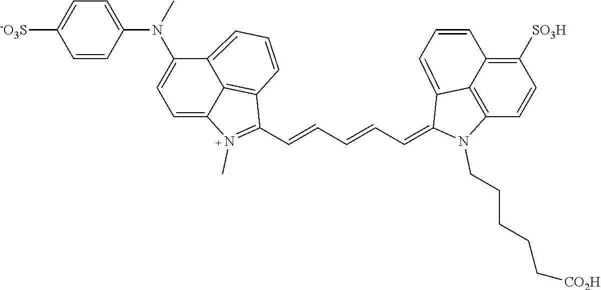 |
| 22 | 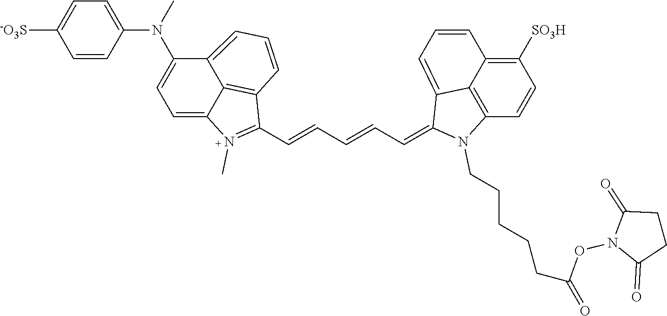 |
| 24 | 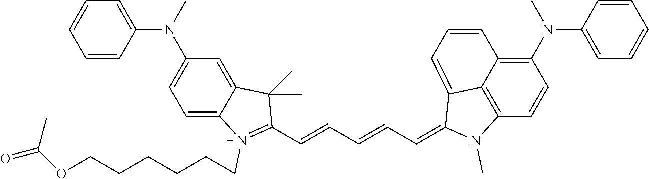 |
| 25 | 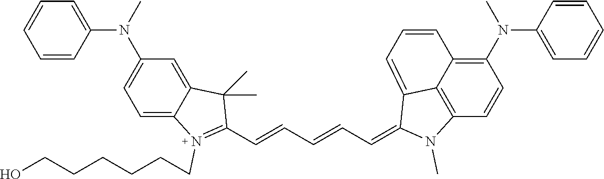 |
| 26 | 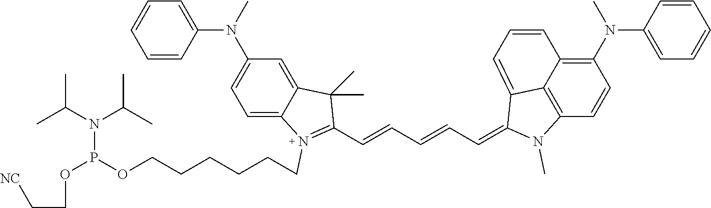 |
| 28 | 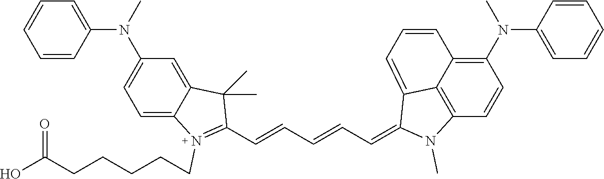 |
| 29 | 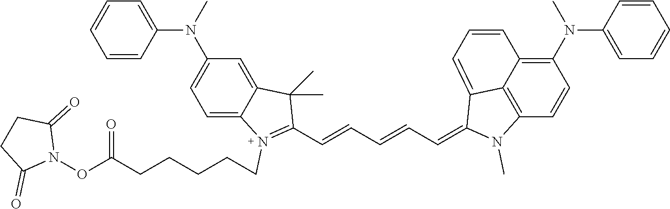 |
| 30 | 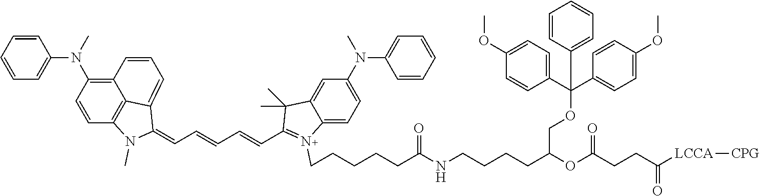 |
| 31 | 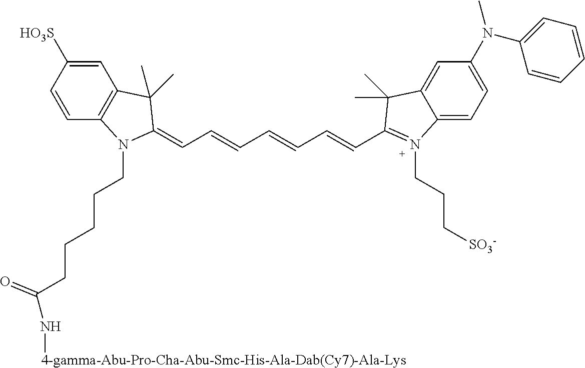 |
| | (SEQ ID NO: 1) |
| | See Example 31 |
| 32 | Arg-Glu(iFluor 750)-Val-Ser-Phe-Asn-Phe-Pro-Gln-Ile-Thr- |
| | Lys(Compound 4)-Arg |
| | (SEQ ID NO: 2) |
| | See example 46 |

### Example 1. Preparation of Compound 1

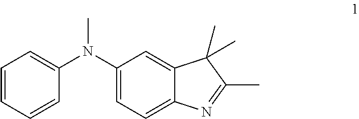 [SMILES: CC1=Nc2ccc(N(C)c3ccccc3)cc2C1(C)C]

Under the dried argon gas protection to a solution of 5-chloro-2,3,3-trimethyl-3H-indole (4 g) in DMF (50 mL), N-methylaniline (4 g) is added, followed by adding Pd(OAc)₂ (0.2 g), JohnPhos (0.6 g) and sodium tert-butoxide (4 g). After 3 hours at 100° C., water (100 mL) is added to quench the reaction at room temperature. The crude product is further purified by HPLC to give the desired Compound 1 as dark brown oil.

### Example 2. Preparation of Compound 2

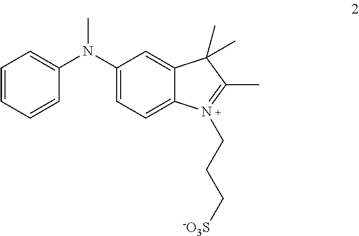

To a solution of Compound 1 (3.6 g) in DMF (50 mL), 1,3-propane sultone (6.6 g) is added. The reaction mixture is heated at 110° C. for 12 hours. The reaction mixture is poured into tetrahydrofuran (200 mL), and the precipitate is collected by filtration, washed with ethyl acetate and dried to give Compound 2 as yellow solid.

### Example 3. Preparation of Compound 3

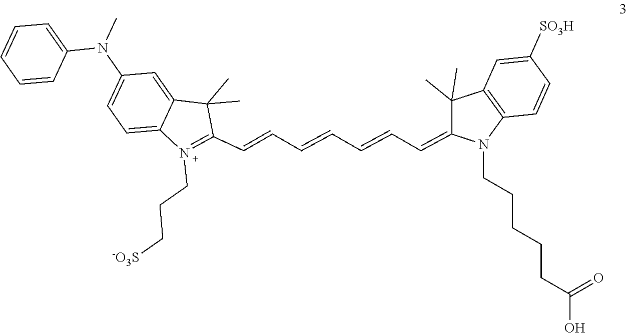

To acetic anhydride (10 mL), 1-(5-carboxypentyl)-2,3,3-trimethyl-3H-indol-1-ium-5-sulfonate (1 g) and N-((1E,3E,5E)-5-(phenylimino) penta-1,3-dien-1-yl) aniline hydrochloride (0.7 g) are added while stirring. The stirred solution is heated to 110° C. and kept at this temperature for 30 minutes. The cooled reaction mixture is poured into ether (100 mL) while stirring. The dark red precipitate is collected by filtration and washed with ether (100 mL). The solid is dried under a high vacuum for several hours. At room temperature, the intermediate is dissolved in DMF (15 mL) and Compound 2 (1 g) is added, followed by adding triethylamine (2.4 mL). The solution is stirred at room temperature overnight. The reaction mixture is poured into ether (50 mL) while stirring. The deep green precipitate is collected by filtration and washed with ether (50 mL). The crude Compound 3 is purified by HPLC to yield the desired pure Compound 3 as a dark green solid.

### Example 4. Preparation of Compound 4

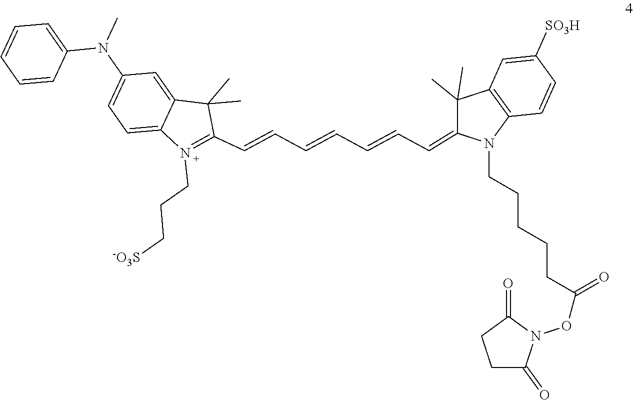

To the solution of compound 3 (0.3 g) in DMF (2 mL), EtN (0.32 mL) and N,N,N′,N′-tetramethyl-O-(N-succinimidyl) uronium tetrafluoroborate (0.12 g) are added. The reaction mixture is stirred at room temperature for 30-60 minutes and poured into ether (50 mL). The deep green precipitate is collected by filtration and washed with ether (50 mL) and dried to give Compound 4 as dark green solid.

### Example 5. Preparation of Compound 5

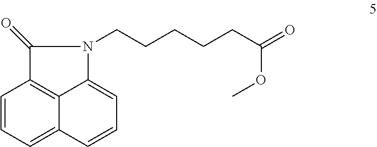 [SMILES: COC(=O)CCCCCN1C(=O)c2cccc3cccc1c23]

To a solution of benzo[cd]indol-2 (1H)-one (5 g) in DMF (50 mL) at 0° C. is slowly added NaH (60% dispersion in mineral, 3.6 g) in portions. The mixture is stirred at 0° C. for 10 min. and followed by the addition of methyl 6-bromohexanoate (7.0 mL). The reaction mixture is stirred from 0° C. to room temperature and kept at room temperature for 2-3 hours. The reaction mixture is quenched with cold saturated NH₄Cl solution, extracted with ethyl acetate, washed with brine and dried with Na₂SO₄. The organic phase is concentrated and purified on a silica gel column (with a gradient of (hexane/ethyl acetate from 9:1 to 8:2) to give pure product Compound 5 as yellow oil.

### Example 6. Preparation of Compound 6

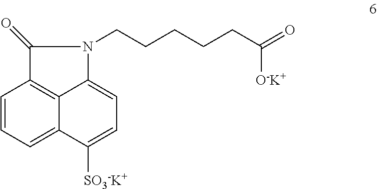

To the solution of Compound 5 (100 mg) in dichloromethane (5 mL) at room temperature is dropwise added chlorosulfonic acid (0.223 mL). The reaction mixture is allowed to stir at room temperature for 1-2 h and quenched with ice water. The quenched reaction mixture is concentrated on a rotavap to remove dichloromethane. To the residue KOH is added to adjust pH to >12. The reaction mixture is stirred at room temperature for a few hours to convert the product to its potassium salt. The crude product is purified on a C18 column with a gradient of 25 mM triethylammonium bicarbonate buffer/MeCN (0-15%) to give Compound 6 as yellow solid.

### Example 7. Preparation of Compound 7

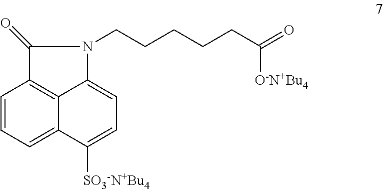

Compound 7 is analogously prepared according to the procedure of Angew. Chem. Int. Ed. 2018, 57, 7483-7487.

### Example 8. Preparation of Compound 8

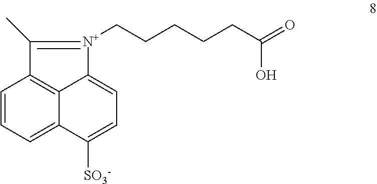

To the solution of Compound 7 (1.0 g) in tetrahydrofuran at 0° C. is slowly added methyl magnesium bromide (3 M in 4 mL ether). The mixture is stirred from 0° C. to room temperature, and heated to 55-60° C. for 1-2 hours. The reaction mixture is quenched with 1 M cold HCl solution. The organic solvent is removed on a rotavap. The aqueous residue is purified on C₁₈ column with a gradient of 0.1% trifluoroacetic acid buffer/MeCN (0-25% MeCN/H₂O) to give the desired Compound 8 as a green solid.

### Example 9. Preparation of Compound 9

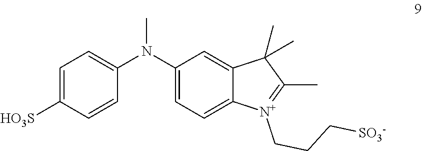

Compound 2 (100 mg) in dichloromethane (3 mL) at 0° C. is added acetic anhydride (0.442 mL) and chlorosulfonic acid (0.052 mL). The mixture is allowed to stir from 0° C. to room temperature and kept at room temperature for 1-2 hours. The reaction mixture is quenched with ice water and concentrated on a rotavap to remove organic solvent. The product is purified by C18 column with a gradient of 0.1% trifluoroacetic acid buffer/MeCN (0-20%) to give desired Compound 9 as green-yellow solid.

### Example 10. Preparation of Compound 10

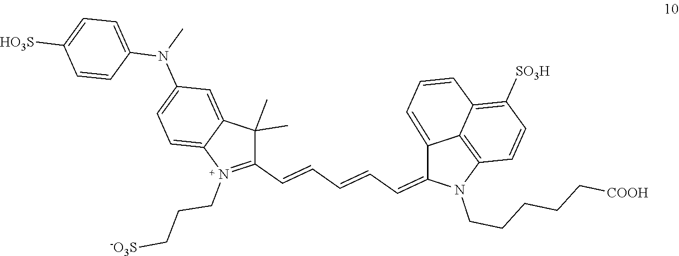

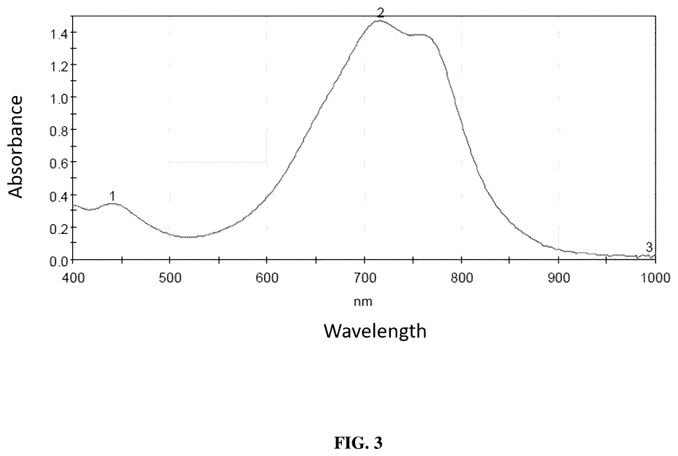

Compound 9 (47 mg) is dissolved in 2 mL of HOAc/AC₂O (1/2, v/v) and heated to 110-115° C. in an oil bath. To the solution, malonaldehyde bis(phenylimine) monohydrochloride (39 mg) is added in portions. The reaction solution is stirred at 110-115° C. for 1-2 hours. The resulting mixture is concentrated under a high vacuum, and the residue is dissolved in DMF. To the DMF solution is added trimethylamine (0.28 ml) and Compound 8 (36 mg). The reaction mixture is stirred at room temperature overnight and concentrated to remove DMF. The residue is treated with ethyl acetate and the precipitate collected. The crude product is dissolved in water and adjusted pH to 8-9. The solution is loaded on a C18 column and eluted with a gradient of 25 mM TEAB buffer/MeCN (0-25%) to give the desired Compound 10. See FIG. 3 for absorbance spectrum.

### Example 11. Preparation of Compound 11

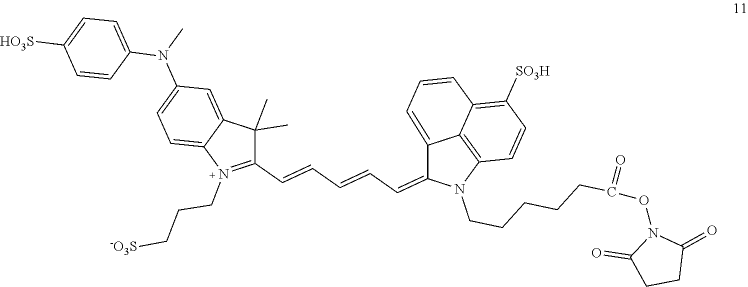

Compound 11 is analogously prepared from Compound 10 according to the procedure of Compound 4 (see Example 4).

### Example 12. Preparation of Compound 12

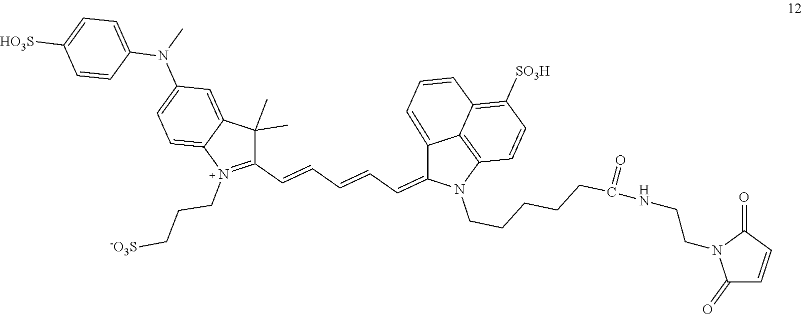

To Compound 11 (10 mg) in DMF (0.2 ml) at room temperature is added triethylamine (0.1 mL) and N-(2-aminoethyl) maleimide, trifluoroacetic acid salt (50 mg, Sigma-Aldrich). The mixture is stirred at ambient temperature for 60 minutes. The DMF solution is poured into ether, and resulted suspension is centrifuged to collect the solid that is air-dried. The crude product is further purified on a C18 column with a gradient of 0.1% trifluoroacetic acid buffer/MeCN (0-20%) to give desired Compound 12 as a blue solid.

### Example 13. Preparation of Compound 13

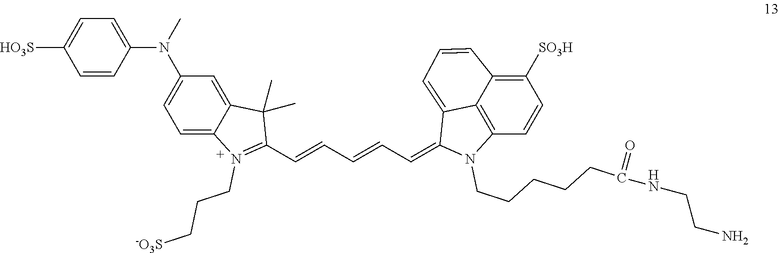

To ethylenediamine (100 mg in 1 ml DMF) is added Compound 11 (10 mg in 0.5 mL DMF) at room temperature. The mixture is stirred at ambient temperature for 60 minutes. The DMF solution is poured into acetone, and resulted suspension is centrifuged to collect the solid that is air-dried. The crude product is further purified on a C18 column with a gradient of 0.1% trifluoroacetic acid buffer/MeCN (0-20%) to give the desired Compound 13 as a blue solid.

### Example 14. Preparation of Compound 14

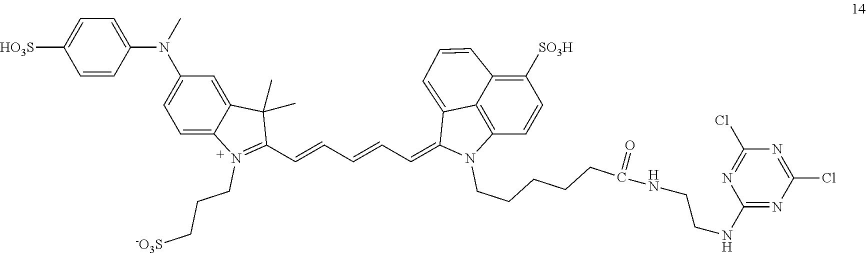

To cyanuric chloride (100 mg in 1 ml DMF) is added Compound 13 (10 mg in 1 mL DMF/pyridine, 1:1) at room temperature. The mixture is stirred at ambient temperature for 60 minutes. The DMF solution is poured into acetone, and resulted suspension is centrifuged to collect the solid that is air-dried. The crude product is further purified on a C18 column with a gradient of 0.1% trifluoroacetic acid buffer/MeCN (0-20%) to give desired Compound 14 as a blue solid.

### Example 15. Preparation of Compound 15

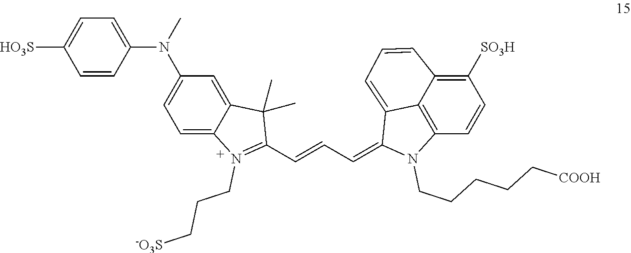

Compound 15 is analogously prepared from Compounds 9 and 8 with the replacement of malonaldehyde bis(phenylimine) monohydrochloride by N,N′-diphenylformamidine according to the procedure of Compound 10 (see Example 10).

### Example 16. Preparation of Compound 16

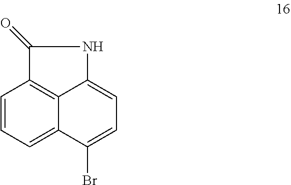 [SMILES: O=C1Nc2ccc(Br)c3cccc1c23]

To the solution of 6-bromobenzo[cd]indol-2 (1H)-one (5.0 g) in chloroform (100 mL) at room temperature is dropwise added bromine (2.3 mL) with a syringe. The reaction mixture is allowed to stir at room temperature for several hours. The reaction mixture is poured into saturated Na₂S₂O₃ solution. The precipitate is collected and washed with water. The residue is dried under a high vacuum to give product the desired Compound 16 as a yellow solid.

### Example 17 Preparation of Compound 17

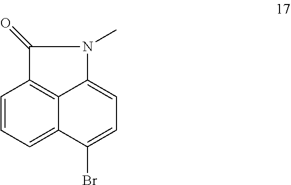

Compound 16 (2.0 g in 30 mL DMF) is added NaH (60% dispersion in mineral, 972 mg) at 0° C. in portions. The resulting DMF solution is stirred from 0° C. to room temperature and followed by the addition of methyl iodide (1.0 mL). The reaction mixture is stirred at room temperature for 1-2 hours and quenched with saturated NH₄Cl solution. The worked-up reaction mixture is extracted with ethyl acetate and washed with brine and dried over anhydrous Na₂SO₄. The ethyl acetate solution is concentrated, and the residue is purified on a silica gel column with a gradient of ethyl acetate/hexanes (from 8:2 to 7:3) to give the desired Compound 17 as a yellow solid.

### Example 18. Preparation of Compound 18

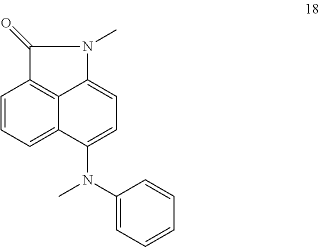 [SMILES: CN1C(=O)c2cccc3c(N(C)c4ccccc4)ccc1c23]

In a Schlenk flask to anhydrous toluene (10 mL) Compound 17 (100 mg), Pd(OAc) 2 (8.5 mg), Xantphos (116 mg) and NaOtBu (73 mg) are added under dry argon. The mixture is degassed and back-filled with argon three times and followed by adding N-methyl aniline (0.081 mL) via a syringe. The reaction mixture is degassed and backfilled with argon three times. Schlenk flask is immersed in a 100° C. oil bath for 4-5 hours. The reaction mixture is concentrated, and the residue is extracted with ethyl acetate and washed with NaHCO₃, brine and dried over anhydrous Na₂SO₄. The ethyl acetate solution is concentrated, and the residue is purified on a silica gel column a gradient of ethyl acetate/hexanes (from 0-25%) to give the desired Compound 18.

### Example 19. Preparation of Compound 19

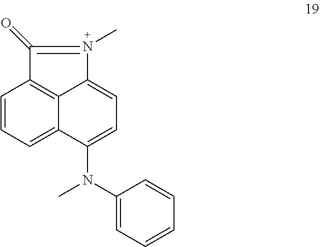

Compound 19 is analogously prepared from Compound 18 according to the procedure of Compound 8 (see Example 8).

### Example 20. Preparation of Compound 20

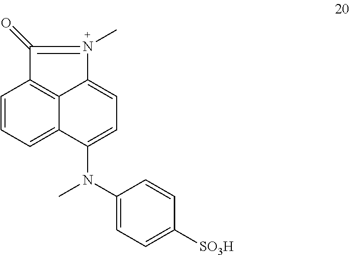

To Compound 19 (100 mg in 2 mL dichloromethane) is added acetic anhydride (0.595 mL) at room temperature. The mixture is cooled to 0° C. and followed by adding chlorosulfonic acid (0.07 mL). The reaction mixture is stirred at 0° C. for 5-10 min and quenched with ice-water. The reaction mixture is concentrated on a rotavap and the residue is purified on a C18 column with a gradient of 0.1% trifluoroacetic acid buffer/MeCN (0-25%) to give the desired Compound 20 as a dark blue solid.

### Example 21 Preparation of Compound 21

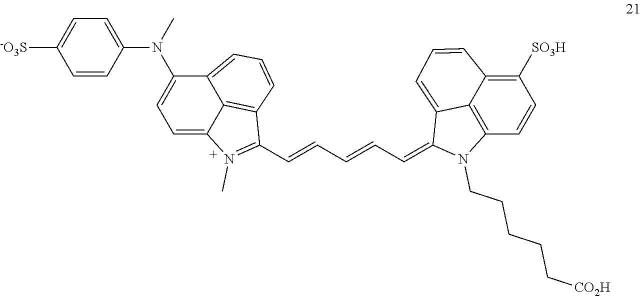

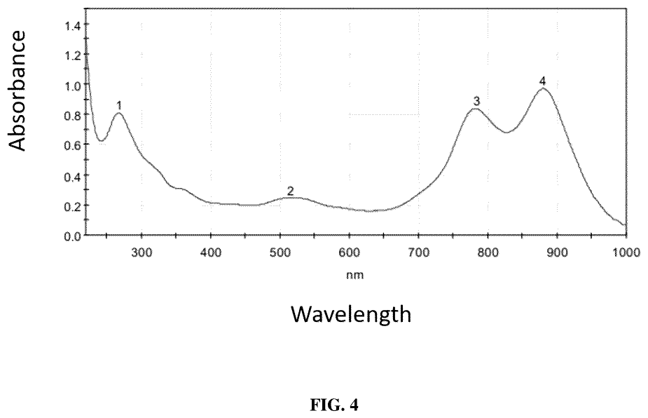

Compound 21 is analogously prepared from Compounds 8 and 20 according to the procedure of Compound 10 (see Example 10). See FIG. 4 for absorption spectrum.

### Example 22. Preparation of Compound 22

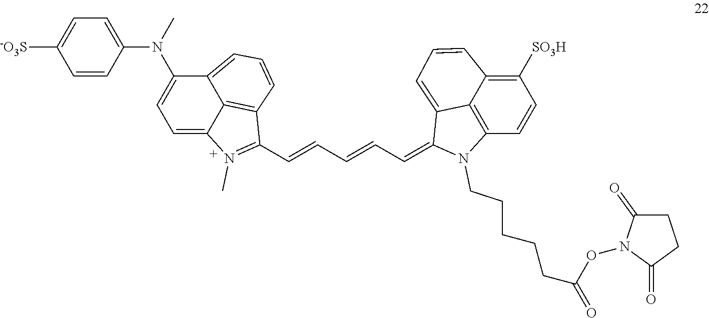

Compound 22 is analogously prepared from Compounds 21 according to the procedure of Compound 4 (see Example 4).

### Example 23. Preparation of Compound 23

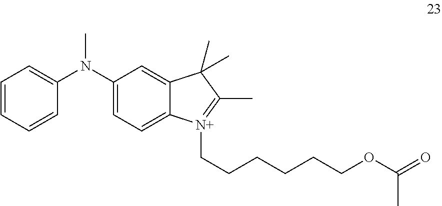 [SMILES: CC(=O)OCCCCCC[N+]1=C(C)C(C)(C)c2cc(N(C)c3ccccc3)ccc21]

Compound 23 is analogously prepared from the quaternization of Compound 1 by 6-bromohexanol acetate according to the procedure of Compound 2 (see Example 2).

### Example 24. Preparation of Compound 24

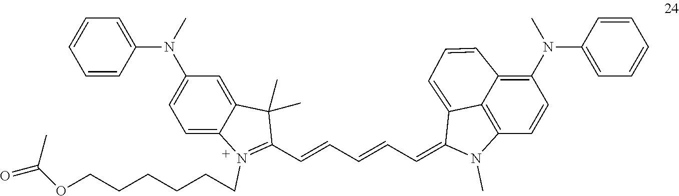

Compound 24 is analogously prepared from the condensation of Compound 19, 23 and malonaldehyde bis(phenylimine) hydrochloride according to the procedure of Compound 13 (see Example 13).

### Example 25. Preparation of Compound 25

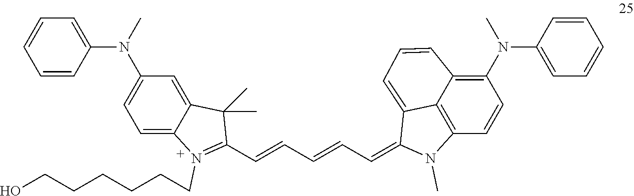

To a solution of Compound 24 (1 g) in MeOH (100 mL), 1M NaOH (5 mL) is added. The reaction mixture is stirred at room temperature for 12 hours. The reaction mixture is neutralized with concentrated HCl. The resulting precipitate is collected by filtration, washed with water and dried to give Compound 25 as a blue solid.

### Example 26. Preparation of Compound 26

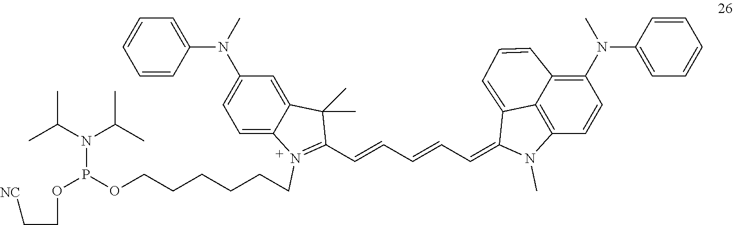

See FIG. 1 for an example phosphoramidite synthesis. To Compound 25 (0.5 g in10 mL dichloromethane) are added tetrazole diisopropylamine (0.3 g) and 2-cyanoethyl N,N,N′,N′-tetraisopropylphosphorodiamidite (0.5 mL). The reaction mixture is stirred at room temperature for 1 hour and diluted with dichloromethane. The mixture is washed with saturated sodium bicarbonate solution and brine. The organic layer is collected, dried with sodium sulfate, filtered and concentrated. The residue is purified by flash chromatography (a gradient of 20-70% hexane-EtOAc with 1% EtaN) to give compound 26 as a blue solid.

### Example 27. Preparation of Compound 27

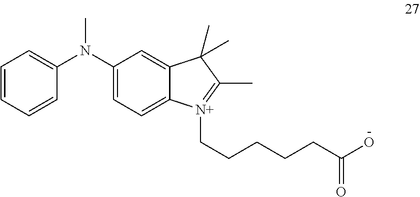 [SMILES: CC1=[N+](CCCCCC(=O)O)c2ccc(N(C)c3ccccc3)cc2C1(C)C]

Compound 27 is analogously prepared from the quaternization of Compound 1 by 6-bromohexanoic acid according to the procedure of Compound 2 (see Example 2).

### Example 28. Preparation of Compound 28

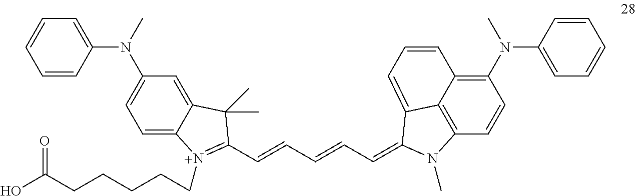

Compound 28 is analogously prepared from the condensation of Compound 19, 27 and malonaldehyde bis(phenylimine) hydrochloride according to the procedure of Compound 13 (see Example 13).

### Example 29. Preparation of Compound 29

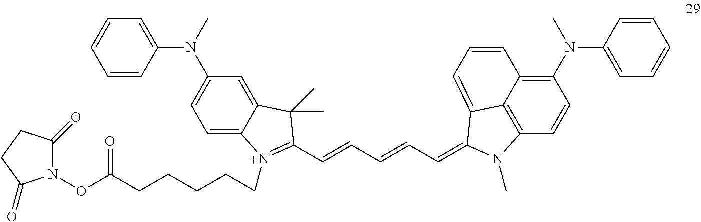

Compound 29 is analogously prepared from Compound 28 according to the procedure of Compound 4 (see Example 4).

### Example 30. Preparation of Compound 30

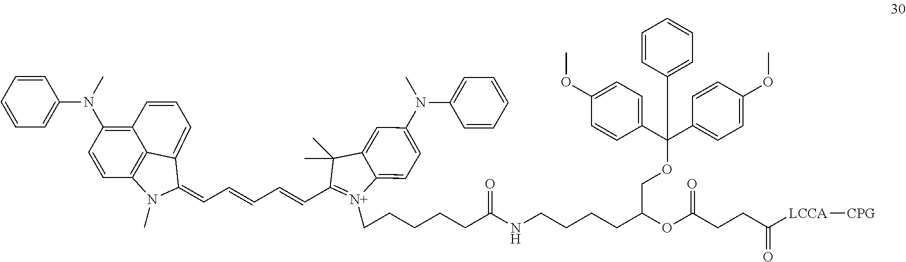

See FIG. 2 for a general schematic preparation. To the solution of N-FMOC-O-DMT-6-amino-1,2-hexanediol (3 g, Xian Biolite Ltd.) and 4-dimethylaminopyridine (200 mg) in anhydrous pyridine (15 mL) is added succinic anhydride (300 mg). The reaction is stirred at room temperature overnight. The consumption of starting material is followed by TLC. The mixture is diluted in ethyl acetate (100 mL), washed with 0.5 M sodium chloride (3×100 mL) and saturated sodium chloride (100 mL), and dried over anhydrous sodium sulfate. After concentrating by rotary evaporation and drying under high vacuum, a yellow solid is obtained. The yellow solid is dissolved in dry dioxane (10 mL) containing anhydrous pyridine (0.5 mL) and p-nitrophenol (350 mg), dicyclohexylcarbodiimide (1.0 g) is added and the mixture is stirred at room temperature. The reaction is monitored by TLC. Upon completion (˜3 hours), the formed dicyclohexylurea is removed by filtration to give the solution of the activated p-nitrophenol ester of N-FMOC-O-DMT-6-amino-1,2-hexanediol-COCH₂CH₂COOH. To the filtrate of the p-nitrophenyl ester derivative, long chain alkylamine CPG (5.0 g, ChemGenes Corp.) and triethylamine (1.0 mL) are added. The suspension is shaken overnight at room temperature. The derivatized support is copiously washed with dimethylformamide, methanol, and diethyl ester and dried in vacuo. Before capping the unreacted alkylamine groups, the loading capacity of the DMT-containing CPG is assayed by determining the amount of dimethoxytrityl cation released upon treatment with perchloric acid according to published procedures (Oligonucleotide Synthesis: A Practical Approach, M. J. Gait (ed.), IRL Press, Oxford, 1984). Finally, the DMT-containing CPG is achieved by treatment with acetic anhydride-pyridine-DMAP (10:90:1. v/v/w) for one hour. The support is thoroughly washed with methanol and diethyl ether and dried under high vacuum to give the desired DMT-containing CPG. The capped CPG gives a negative ninhydrin test. The FMOC group of the capped CPG is cleaved as described in the art (U.S. Pat. No. 5,401,837 to P. S. Paul et al.). The FMOC-cleaved long chain alkylamine (LCCA)-controlled pore glass (CPG) (5.0 g) is suspended in 10 ml of DMF solution containing Compound 29 (500 mg) and N,N-diisopropylethylamine (1.0 ml), and the mixture is shaken overnight at room temperature. The derivatized support is copiously washed with dimethylformamide, methanol, and diethyl ether and dried in vacuo. Before capping the unreacted alkylamine groups, the loading capacity of the dye-labeled CPG is assayed by determining the amount of dimethoxytrityl cation released upon treatment with perchloric acid according to published procedures (Oligonucleotide Synthesis: A Practical Approach, M. J. Gait (ed.), IRL Press, Oxford, 1984). Finally, capping of the dye-labeled CPG is achieved by treatment with acetic anhydride-pyridine-DMAP (10:90:1. v/v/w) for one hour. The support is thoroughly washed with methanol and diethyl ether and dried under high vacuum to give the dye-labeled CPG that give a negative ninhydrin test.

### Example 31. Synthesis of a Cyanine Dye-Labeled FRET Peptide

gamma-Abu-Pro-Cha-Abu-Smc-His-Ala-Dab (Cy7)-Ala-Lys-NH₂ (SEQ ID NO: 3) is synthesized by the standard FMOC solid phase synthesis as described in Standard practices for Fmoc-Based Solid-Phase Peptide Synthesis (Version 1.7.2), Adam G. Kreutzer, A. G. et al.

(chem.uci.edu/˜jsnowick/groupweb/files/Standard_practices_for_Fmoc_based_solid_ph ase_peptide_synthesis_in_the_Nowick_Laboratory_V_1.7.2.pdf). To gamma-Abu-Pro-Cha-Abu-Smc-His-Ala-Dab (Cy7)-Ala-Lys-NH₂ (SEQ ID NO: 3) (10 mg in 1 ml DMF) is added NHS ester Compound 4 (10 mg in 0.5 mL DMF) at room temperature. The mixture is stirred at ambient temperature for 60 minutes. The DMF solution is concentrated under high vacuum, and the residue purified on a C₁₈ column with a gradient of 0.1% trifluoroacetic acid buffer (0-20% MeCN/H₂O) to give the desired peptide Compound 4-gamma-Abu-Pro-Cha-Abu-Smc-His-Ala-Dab (Cy7)-Ala-Lys (Compound 4)-NH₂ (SEQ ID NO: 4) as a blue solid. A variety of labelled peptides can be prepared using solid phase synthesis methods and where needed, orthogonal sidechain protecting groups and methods to install quenchers and dyes at particular positions of a target polypeptide sequence.

### Example 32. Synthesis of a Cyanine Dye-Labeled Oligonucleotide Using a Cyanine Dye Phosphoramidite

Oligonucleotide synthesis is performed using an automated DNA synthesizer according to manufacturer's instructions. Compound 26 is used to label oligonucleotides. In each final coupling cycle, the Trityl ON configuration is used. After assembly, the oligonucleotides are cleaved from the support using concentrated ammonia using the manufacturer's end procedure cycle. The residue is dissolved in acetic acid/water (8:2) and the mixture is evaporated to dryness after 20 minutes at room temperature. To the residue is added water (0.5 ml) and the resultant suspension filtered. The aqueous solution now contains the deprotected oligonucleotide ready for purification. Compound 26 (100 mg) is dissolved in 1 mL of dry acetonitrile and placed on the DNA synthesizer. Following the procedure suggested by the manufacturer, 50 μL of the solution of Compound 26 is delivered to the reaction column with 100 μL of a 0.5 M tetrazole activator solution. The mixture is cycled over the support containing the 5′-OH oligonucleotide for a few minutes. Following the removal of excess Compound 26, the typical coupling cycle is completed by oxidiation, capping, and detritylation. Labeled oligonucleotides (5 mer to 10 mer lengths) are released from the solid support and deprotected by treating with concentrated ammonium hydroxide for 20 minutes at 60° C. The cyanine dye-labeled oligonucleotide are purified and analyzed by TLC (Kieselgel 60 F254 in 55:10:35 isopropanol: water: ammonia) or by reverse phase HPLC (gradient of 10-40% A in B over 30 minutes; A=acetonitrile, B=0.1M triethylammonium acetate, pH 7) or by polyacrylamide gel electrophoresis according to standard procedures (Sambrook et al., Molecular Cloning: A Laboratory Manual, 2nd Edition, Cold Spring Harbor Laboratory Press, 1989).

### Example 33. Synthesis of a Cyanine Dye-Labeled Oligonucleotide Using a Dye CPG Polymer

Oligonucleotide synthesis is performed using an automated DNA synthesizer according to manufacturer's instructions. Compound 30 is used to label oligonucleotides. Cyanine dye controlled pore glass (CPG) supports (such as Compound 30) are used for attachment of quencher dyes to the 3′ terminus of nucleic acids. 5′ fluorophore labeling is accomplished using fluorophore phosphoramidites. Cleavage and deprotection of cyanine dye oligos is carried out in ammonia at 60° C. Following deprotection, dual labeled probes are purified by HPLC. Purified probes are analyzed by both anion exchange and reverse phase HPLC. The dual labeled FRET oligonucleotides are analyzed and used as Melecular Beacons as described in the art (D. P. Bratu (2006), Methods Mol Biol 319, 1-14; S. A. E. Marras (2006), Methods Mol Biol 335, 3-16; S. A. E. Marras, S. Tyagi, and F. R. Kramer (2006), Clin Chim Acta 363, 48-60; A. P. Silverman and E. T. Kool (2005), Trends Biotechnol 23, 225-230).

### Example 34. Preparation of Oligonucleotide Conjugates Using a Quenching Compound

Eighteen-base oligonucleotide conjugates of quencher dyes are prepared using standard methods. Typically, a 6-(N-trifluoroacetylamino) hexyl is synthetically incorporated on the 5′ end of the oligonucleotide of interest as a phosphoramidite, and the trifluoroacetic acid protection group is then removed under basic conditions. The resulting conjugate is subsequently reacted with a succinimidyl ester derivative of a quenching compound of the disclosure. Specifically, the succinimidyl ester derivative of a cyanine dye (such as Compounds 4, 11, 22 and 29) is dissolved in DMSO at a concentration of about 12.5 mg/mL. The amine-modified oligonucleotide is dissolved in water at a concentration of 25 mg/mL. A fresh solution of 0.1 M sodium borate, pH 8.5 is prepared. In a microfuge tube, 4 μL of the oligonucleotide solution is combined with 200 μg of the quenching compound solution and 100 μL sodium borate buffer. Samples are incubated 4 hours to overnight at room temperature, and the nucleic acids are precipitated by addition of 0.1 volume 0.3 M NaCl and 2.5 volumes cold absolute ethanol. Samples are incubated for 30 minutes at −20° C. and centrifuged in a microfuge for 30 minutes. The supernatant fluid is decanted, and the pellet dried under vacuum. Alternatively, the oligonucleotide conjugate may be prepared by reaction of a maleimide derivative of a quenching compound of the disclosure with an oligonucleotide that has been derivatized by a thiol that has been incorporated via a phosphoramidite. Conjugates may be purified by reverse phase HPLC, using a C₁₈ reverse phase column and a gradient of 5-95% acetonitrile in 0.1 M TEAA, pH 7. Absorbance and fluorescence emission spectra are determined in 10 mM Tris-HCl, 1 mM EDTA, pH 7.5. Oligonucleotide conjugates of quenching compounds may be weakly fluorescent or substantially non-fluorescent.

### Example 35. Preparation of an Oligonucleotide Conjugate, Substituted with Both a Fluorophore and a Quenching Compound

Oligonucleotides conjugated to a fluorophore at one terminus and a quenching compound of the disclosure at the other terminus are prepared using a cyanine quencher phosphoramidite and an amine modifier at the other terminus of the oligonucleotide, followed by labeling with a succinimidyl ester derivatives of the disclosure, or by synthesis of oligonucleotides containing an amino modifier on one terminus and a thiol at the other terminus, followed by sequential reaction with a maleimide derivative and succinimidyl ester derivative of the fluorophore and quenching compound, or vice versa. The fluorescence of the resulting conjugates is measured at equal conjugate concentration. Selected compounds of the instant disclosure quench the fluorescence of longer wavelength fluorophores much more efficiently than DABCYL and other existing quenchers.

### Example 36. Hybridization of Doubly-Labeled Oligonucleotide Conjugates to Unlabeled Complementary Oligonucleotides

Solutions are prepared containing 1 μg/mL 18-base oligonucleotide conjugates of a quenching compound of the disclosure attached to the 5′ terminus, as well as a fluorophore on the 3′ terminus. The oligonucleotide conjugates are hybridized with 40 μg/ml reverse complement oligonucleotide in TE buffer at pH 9.0. The samples are heated for 10 minutes at 65° C., allowed to cool slowly to room temperature, and are then incubated at room temperature for 60 minutes, protected from light. A portion of each sample is transferred to a microplate well and the fluorescence emission of the sample is determined. The fluorescence is compared to the fluorescence of a buffer solution alone. The conjugates of the disclosure exhibit an increase in fluorescence upon hybridization. Because the quenched oligonucleotides initially exhibited extremely low fluorescence, they show larger increases upon hybridization, and therefore the conjugates that are the most efficiently quenched prior to hybridization typically exhibit the largest increase in fluorescence. This property may be utilized to formulate a homogenous assay method to detect the presence of the specific complementary DNA sequences in a sample. Several of the compounds of the disclosure quench IR fluorescence more efficiently than DABCYL other existing quenchers in this application. Similarly, doubly labeled oligonucleotides that form structures that enhance quenching, such as hairpin or stem loop structures, as in BEACON probes, can also be used in this application.

### Example 37. Hybridizing Oligonucleotide Conjugates of Quenching Compounds with Fluorophore Labeled Oligonucleotides

Oligonucleotides conjugated to a quenching compound at one terminus quench the fluorescence of fluorophore labeled nucleotides upon hybridization. Labeled oligonucleotides are prepared as described above (Examples 32 and 33), and hybridized with their reverse complements. Samples containing 2 μg/mL quenching compound-labeled 18 base oligonucleotides and 200 ng/ml Cy3-labeled reverse complement oligonucleotides in 10 mM Tris-HCl, 1 mM EDTA, pH 9.0, are hybridized and their fluorescence is determined as described above (Example 34). The quenching compound oligonucleotides efficiently quench the fluorescence of the fluorophore that is localized at the same end of hybridized oligonucleotides, but quench the fluorescence of distant fluorophores more poorly.

### Example 38. Quenching Fluorescence of Nucleotides Added Enzymatically to the 3′ End of a Primer

An eighteen-base oligonucleotide is labeled with Compound 4 on its 5′ terminus, as described in Example 33. The resulting conjugate is incubated with terminal deoxynucleotidyl transferase under standard assay conditions for 3′ end elongation, in the presence of iFluor 750-labeled dUTP conjugates, as follows: The oligonucleotide conjugate (650 ng) is incubated with 1 μL of 25 mM iFluor 750-labeled nucleotide, 0.5 mM CoCl₂, and 0.2 M potassium cacodylate, 25 mM Tris-HCl, pH 6.6, 2 mM DTT, and 250 μg/mL bovine serum albumin for 60 minutes at 37° C. A one-fifth volume of a solution containing 50% glycerol and 0.01% bromophenol blue is added to each reaction, and the samples are separated by electrophoresis on a 20% polyacrylamide/8 M urea minigel in TBE buffer (45 mM Tris-borate, 1 mM EDTA), under conditions that resolve single nucleotide additions to the oligonucleotide. Samples containing oligonucleotides that are lacking the quenching compound are processed in parallel, for use as size standards. Gels are visualized using a 300-nm UV transilluminator combined with Polaroid black and white photography, or using a laser scanner. The gels are post-stained with a fluorescent nucleic acid stain, such as ethidium bromide, and band fluorescence is visualized in the same way. The size of the oligonucleotides is determined based on comparisons of electrophoretic migration with the unlabeled standard. Quenching is detected as lack of fluorescence or visibility of a band of a particular size from the pattern visible in the standard. Where the fluorophore is iFluor 750 dye, the label fluorescence is readily quenched by the 5′-bound quenching compound. This technique is useful as a gel-based method for quantitating terminal transferase activity. Enzyme activity in an unknown sample is determined by comparison of the number of added nucleotides per template or the number of templates with added nucleotides of a certain length with the numbers obtained using a standard amount of enzyme activity following a standard reaction time interval.

### Example 39. Quenching of a Fluorescent Oligonucleotide by Enzymatic Incorporation of a Quenching Compound Conjugate of Nucleotide Triphosphate Via Primer Extension

A short oligonucleotide, having 6 to about 20 bases, is labeled with a fluorophore such as iFluor 647 dye, on its 5′ terminus, and then purified via HPLC. For template-driven reactions, the oligonucleotide is hybridized to an appropriate template, and incubated with Compound 29-labeled nucleotide or deoxynucleotide in an appropriate buffered solution, in the presence of samples thought to contain an appropriate DNA or RNA polymerase. Enzyme activity is determined by measuring the rate of fluorescence loss from the solution, versus the rate of loss observed from solutions containing known amounts of enzyme activity. Terminal deoxynucleotidyltransferase activity is assayed by determining the rate of fluorescence loss from the solution upon incubation with samples thought to contain terminal deoxynucleotidyltransferase activity. For measurement of terminal deoxynucleotidyl transferase activity, fluorophore-labeled templates are incubated with quenching compound-labeled nucleotides for a set time interval, and fluorescence is measured in a fluorescence microplate reader or fluorometer.

### Example 40. Measuring Reverse Transcriptase Activity Using a Quencher-Labeled Nucleotide

To measure reverse transcriptase activity, 2 μg mRNA is combined with 5 μg iFluor 647 labeled poly dT (16) oligomer in 10 mM Tris-HCl, pH. 8.0, 1 mM EDTA; the mixture is heated to 70° C. for 10 minutes and then chilled on ice. A solution containing 2 μL reverse transcriptase (200 units/μL for the standard, or unknown amounts), 500 uM dATP, 500 UM dCTP, 500 uM dGTP, 200 uM dTTP, and 60 uM Compound 29-labeled dUTP is prepared and added to the RNA. The reaction is allowed to proceed for 2 hours at 42° C. The fluorescence of the solution is measured in a fluorescence microplate reader or fluorometer versus a standard. The decrease in fluorescence in comparison to samples lacking enzyme activity is directly related to the activity of the enzyme in the reaction.

### Example 41. Measuring Polymerase Activity Using a Quencher-Labeled Nucleotide

To measure Klenow DNA polymerase activity, 1 μg random sequence 9-mer oligonucleotides labeled with iFluor 647 are combined with 2.5 μg genomic DNA in 10 mM Tris-HCl, pH 8.0, 1 mM EDTA. The mixture is boiled for 2 minutes and chilled on ice. A reaction mixture containing 25 uM dATP, 25 uM dCTP, 25 uM dGTP and 10 UM dTTP, plus 40 uM Compound 29-labeled dUTP in 1 mM Tris-HCl, pH 7.5, 5 mM NaCl, 0.01 mM EDTA, pH 8.0, 5 mM dithiothreitol is combined with samples thought to contain DNA polymerase. The reaction mixture is combined with the DNA mixture and incubated at 37° C. for 2 hours.

### Example 42. Using Quenching Compounds to Measure Nuclease Activity

Oligonucleotide conjugates labeled with both a quenching compound at one terminus and a fluorophore at the other terminus are prepared as described in Examples 17 and 18. For measuring single-stranded nuclease activity, the conjugates are incubated in the presence of samples thought to contain nuclease activity in the presence of an appropriate buffer and the resulting fluorescence increase in the sample is compared to that obtained using standards of known nuclease concentration. To measure double-stranded nuclease activity, double-stranded templates are prepared by hybridizing two oligonucleotides to one another, or by chemically modifying a double-stranded template using reagents such as platinum complexes of fluorophores and quenchers (as described in U.S. Pat. Nos. 5,714,327 and 6,133,038, incorporated by reference), or by using an enzyme such as a terminal transferase to add nucleotides to the end of a template as described in Examples 32 and 33. Samples thought to contain nuclease activity are incubated with such templates in the presence of appropriate buffers and the increase in fluorescence compared to a standard, as described in Example 34.

### Example 43. Using Quenching Compounds to Measure Ligase Activity

Oligonucleotide hexamers labeled at the 5′ terminus with a quenching compound are prepared as described in Example 32 and 33. Oligonucleotide hexamers labeled with a fluorophore at the 3′ terminus and phosphate at the 5′ terminus are analogously prepared except that the phosphate is alternatively applied by standard methods using a phosphoramidite or by enzymatic means, such as T4 polynucleotide kinase. A reaction mixture is prepared that contains about 5 μg of each oligonucleotide conjugate, 0.5 mM ATP, and samples thought to contain ligase activity, in 1 mM MgCl2, 2 mM dithiothreitol, 5 μg/ml bovine serum albumin, and 5 mM Tris-HCl, pH 7.7, in a volume of 20 μl. The reaction mixtures are incubated for 2 hours to overnight at 22° C., and the sample fluorescence is measured. As the quenching compound-labeled oligonucleotides do not contain a free 5′ phosphate, they cannot ligate to one another, and as the fluorophore-labeled oligonucleotides do not contain a free 3′ hydroxyl, they cannot ligate to one another. Thus the only products of ligation will be a dimer of the two oligonucleotides and the fluorescence decrease observed during the course of the reaction is a measure of ligase activity. Alternatively, RNA oligonucleotides are used as templates to measure RNA ligase activity or splicing activity.

### Example 44. Using Quenching Compounds to Assay Topoisomerase Activity

Quenched DNA is prepared as described above, using a circular single stranded DNA template, such as an M13 or ØX174 phage DNA genome, and a quenching compound platinum complex (prepared as described in U.S. Pat. No. 5,714,327). A fluorophore-labeled oligonucleotide is then hybridized to the quenched DNA. Samples thought to contain topoisomerase activity are combined with the template under optimal reaction conditions for the enzyme, and the reaction is allowed to proceed for an appropriate period of time. Enzyme activity is measured as fluorescence increase for the solution, using a fluorescence microplate reader or fluorometer.

### Example 45. Detection of Matrix Metalloproteinase Activities

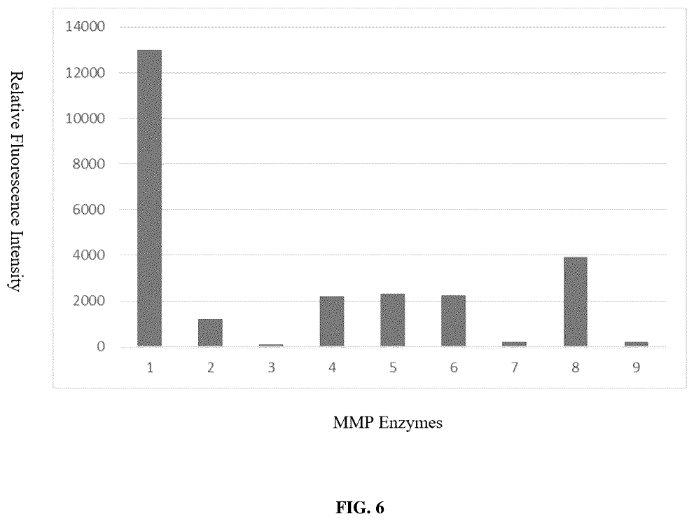

The matrix metalloproteinases (MMPs) constitute a family of zinc-dependent endopeptidases that function within the extracellular matrix. These enzymes are responsible for the breakdown of connective tissues and are important in bone remodeling, the menstrual cycle and repair of tissue damage. While the exact contribution of MMPs to certain pathological processes is difficult to assess, MMPs appear to have a key role in the development of arthritis as well as in the invasion and metastasis of cancer. 50 AM the FRET peptide is incubated with 4 nM MMPs or without MMPs (control) at room temperature. The pro-MMPs are activated with 2 mM APMA. The activate MMPs is incubated with the FRET substrate (see Example 31) at a desired temperature for the enzyme reaction (e.g. 25° C. or 37° C.) for 10-15 minutes. The fluorescence intensities are recorded with a fluorescence plate reader at Ex/Em=750/780 nm. The recording is started as soon as the enzymatic reaction is initiated. The result is shown in FIG. 6.

### Example 46. Detection of HIV Protease Activities Using FRET Peptides

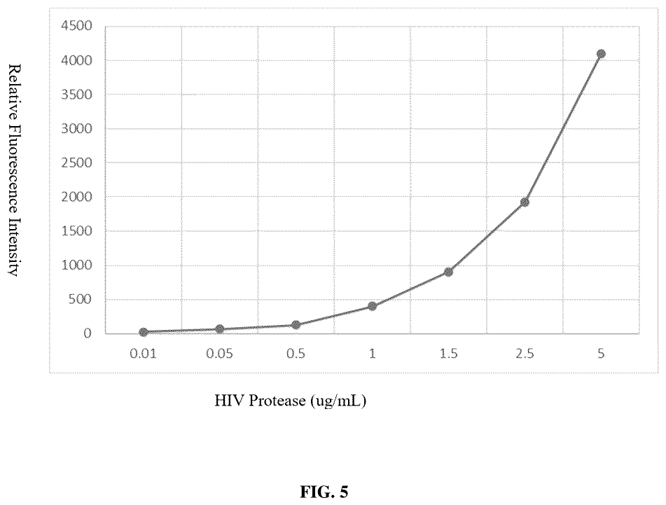

Inhibition of HIV-1 protease represents an important avenue for AIDS therapy. Currently combination chemotherapy of reverse transcriptase inhibitors and protease inhibitors have shown to suppress the replication of HIV-1 and extend the life expectancy of HIV-1-infected individuals. Arg-Glu (iFluor 750)-Val-Ser-Phe-Asn-Phe-Pro-Gin-Ile-Thr-Lys (Compound 4)-Arg, (SEQ ID NO: 2) a FRET peptide of the instant disclosure, is used as a fluorogenic HIV protease substrate for detecting HIV protease activity. As shown in FIG. 5. The FRET peptide demonstrates excellent response to HIV-1 protease.

### Equivalents and Incorporation by Reference

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

All references to issued patents and patent applications as well as non-patent documents cited within the body of the instant specification are hereby incorporated by reference in their entirety for all purposes.

1. A compound having Formula I:
WSM−{X—[CR₁═CR₂]ₙ—CR₃═Y}-RM Formula I
wherein:
n is an integer from 0 to 3;
X is an optionally substituted moiety selected from indolium, pyrrolium, oxazolium, a thiazolium, imidazolium, pyridinium, and quinolinium;
Y is an optionally substituted moiety selected from indoline, a pyrrole, oxazoline, a thiazoline, imidazoline, pyridine, and quinoline;
R₁ is H, halogen, or an optionally substituted moiety selected from alkyl, aryl, thiol, amino, and heterocycle;
R₂ is H, halogen, or an optionally substituted moiety selected from alkyl, aryl, thiol, amino, and heterocycle;
R₃ is H, halogen, or an optionally substituted moiety selected from alkyl, aryl, thiol, amino, and heterocycle;
RM is a chemically reactive moiety; and
WSM is a water-soluble moiety;
provided that at least one of X and Y contain an optionally substituted arylamino or heteroarylamino group.

2. The compound of claim 1, wherein:
X is an optionally substituted indolium or quinolinium moiety;
Y is an optionally substituted indoline or quinoline moiety;
each of R₁, R₂, and R₃ are H;
RM is an activated ester; and
WSM is an optionally substituted sulfonate or phosphonate.

3. The compound of claim 1, wherein X is of Formula II: 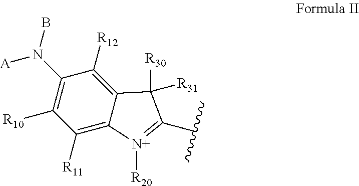
wherein:
A and B are each independently H or an optionally substituted alkyl, alkyl, aryl, aryl, heteroaryl, or heteroaryl;
R₁₀ to R₁₂ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boronic acid, aryl, and heteroaryl;
R₂₀ is an optionally substituted alkyl, aryl, or heteroaryl; and
R₃₀ and R₃₁ are each independently an optionally substituted moiety selected from alkyl, aryl, and heteroaryl;
provided that at least one of A or B is aryl or heteroaryl.

4. The compound of claim 3, wherein:
A and B are independently H, or an optionally substituted alkyl or phenyl;
R₁₀ to R₁₂ are independently H, halogen, or an optionally substituted moiety selected from carboxy, alkyl, alkoxy, aryloxy, thiol, alkylthiol, arylthiol, aryl, and heteroaryl;
R₂₀ is an optionally substituted alkyl, aryl or heteroaryl; and
R₃₀ and R₃₁ are independently optionally substituted alkyl or aryl.

5. The compound of claim 4, wherein:
A and B are each independently H, or an optionally substituted sulfonated alkyl, or a sulfonated phenyl;
R₁₀ to R₁₂ are each independently H, halogen, or an optionally substituted moiety selected from carboxy, alkyl, alkoxy, aryloxy, aryl, and heteroaryl;
R₂₀ is a moiety selected from an optionally substituted alkyl, sulfonated alkyl and carboxylated alkyl; and
R₃₀ and R₃₁ are independently alkyl.

6-8. (canceled)

9. The compound of claim 1, wherein X is of Formula III 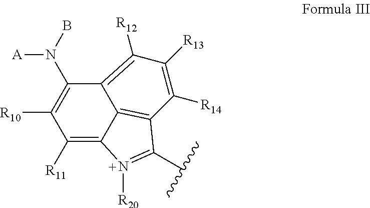
wherein:
A and B are each independently H, or an optionally substituted moiety selected from alkyl, aryl, and heteroaryl;
R₁₀ to R₁₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl; and
R₂₀ is an optionally substituted alkyl, aryl or heteroaryl;
provided that at least one of A and B is an aryl or a heteroaryl.

10. The compound of claim 9, wherein:
A and B are independently H, or an optionally substituted moiety selected from alkyl and phenyl;
R₁₀ to R₁₄ are each independently H, halogen, or an optionally substituted moiety selected from carboxy, alkyl, alkoxy, aryloxy, thiol, alkylthiol, arylthiol, aryl, and heteroaryl; and
R₂₀ is an optionally substituted alkyl, aryl, or heteroaryl.

11. (canceled)

12. A compound having Formula IV:
{X—[CR₁═CR₂]ₙ—CR₃═Y}—CPG Formula IV
wherein:
n is an integer from 0 to 3;
X is an optionally substituted moiety selected from indolium, pyrrolium, oxazolium, a thiazolium, imidazolium, pyridinium, and quinolinium;
Y is an optionally substituted moiety selected from indoline, pyrrole, oxazoline, thiazoline, imidazoline, pyridine, and quinoline;
R₁ to R₃ are each independently H, halogen, or an optionally substituted moiety selected from alkyl, aryl, thiol, amino, and heterocycle; and
CPG is a controlled pore glass as solid support for oligo nucleotide synthesis;
provided that at least one of X and Y is an arylamino or a heteroarylamino group.

13. The compound of claim 12, wherein:
X is an optionally substituted indolium or quinolinium moiety;
Y is an optionally substituted indoline or quinoline moiety; and
R₁ to R₃ are each H.

14. The compound of claim 12, wherein X is of Formula II 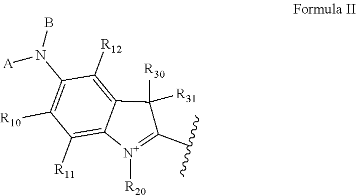
wherein:
A and B are each independently H, or an optionally substituted moiety selected from alkyl, aryl, and heteroaryl;
R₁₀ to R₁₂ are each independently H, halogen, or an optionally substituted carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ is an optionally substituted alkyl, aryl, or heteroaryl;
R₃₀ and R₃₁ are each independently optionally substituted alkyl, aryl, or heteroaryl provided that at least one of A and B is aryl or heteroaryl.

15. (canceled)

16. The compound of claim 12, wherein X is of Formula III 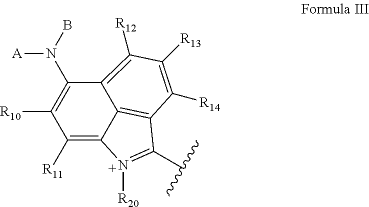
wherein:
A and B are each independently H, or an optionally substituted moiety selected from alkyl, aryl, and heteroaryl;
R₁₀ to R₁₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ is an optionally substituted alkyl, aryl, and heteroaryl;
provided that at least one of A and B is aryl or heteroaryl.

17. The compound of claim 16, wherein:
A and B are each independently H, or an optionally substituted alkyl or phenyl;
R₁₀ to R₁₄ are each independently H, halogen, or an optionally substituted moiety selected from carboxy, alkyl, alkoxy, aryloxy, thiol, alkylthiol, arylthiol, aryl, and heteroaryl; and
R₂₀ is an optionally substituted alkyl, aryl or heteroaryl.

18. A compound having Formula V: 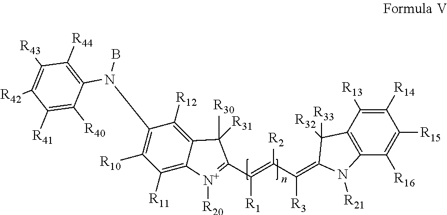
wherein:
B is H, or an optionally substituted alkyl, aryl, or heteroaryl;
n is an integer from 0 to 3;
R₁ to R₃ are each independently H, halogen, or an optionally substituted moiety selected from alkyl, aryl, thiol, amino, and heterocycle;
R₁₀ to R₁₆ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ and R₂₁ are each independently an optionally substituted alkyl, aryl or heteroaryl; and
R₃₀ to R₃₃ are each independently an optionally substituted alkyl, aryl, or heteroaryl;
provided that at least there is a water soluble moiety and a reactive moiety.

19. The compound of claim 18, wherein:
B is H, or an optionally substituted alkyl or phenyl;
n is an integer from 1 to 3;
R₁ to R₃ are H;
R₁₀ to R₁₆ and R₄₀ to R₄₄ are each independently H, halogen, or an optionally substituted moiety selected from carboxy, alkyl, alkoxy, aryloxy, thiol, alkylthiol, arylthiol, sulfonate, aryl, and heteroaryl;
R₂₀ and R₂₁ are each independently an optionally substituted alkyl, carboxylated alkyl or sulfonated alkyl; and
R₃₀ to R₃₃ are an optionally substituted alkyl;
provided that at least there are two sulfonates and a reactive moiety.

20. (canceled)

21. A compound having Formula VI: 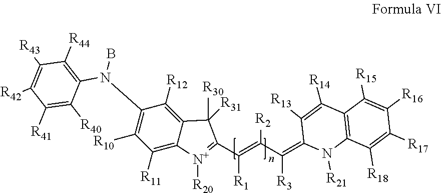
wherein:
B is H, or an optionally substituted alkyl, aryl or heteroaryl;
n is an integer from 0 to 3;
R₁ to R₃ are each independently H, halogen, or an optionally substituted moiety selected from alkyl, aryl, thiol, amino, and heterocycle;
R₁₀ to R₁₈ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ and R₂₁ are each independently optionally substituted alkyl, aryl, or heteroaryl;
R₃₀ and R₃₁ are each independently an optionally substituted alkyl, aryl or heteroaryl;
provided that at least there is a reactive moiety and a water soluble moiety.

22. The compound of claim 21, wherein:
B is H, an optionally substituted alkyl or phenyl;
n is an integer from 1 to 3;
R₁ to R₃ are H;
R₁₀ to R₁₈ and R₄₀ to R₄₄ are each independently H, halogen, or an optionally substituted moiety selected from carboxy, alkyl, alkoxy, aryloxy, sulfonate, thiol, alkylthiol, arylthiol, aryl, and heteroaryl;
R₂₀ and R₂₁ are each independently optionally substituted alkyl, carboxylated alkyl or sulfonated alkyl; and
R₃₀ and R₃₁ are optionally substituted alkyl, provided that at least there are two sulfonates and a reactive moiety.

23. (canceled)

24. A compound having Formula VII: 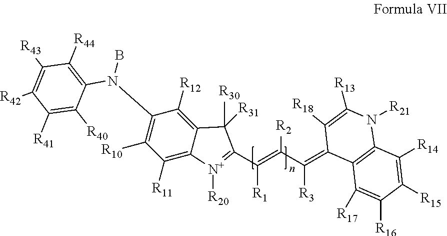
wherein:
B is H, or an optionally substituted moiety selected from alkyl, aryl and heteroaryl;
n is an integer from 0 to 3;
R₁ to R₃ are each independently H, halogen, or an optionally substituted moiety selected from alkyl, aryl, thiol, amino, and heterocycle;
R₁₀ to R₁₈ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ and R₂₁ are each independently optionally substituted alkyl, aryl, or heteroaryl; and
R₃₀ and R₃₁ are each independently optionally substituted alkyl, aryl, or heteroaryl;
provided that at least there is a reactive moiety and a water soluble moiety.

25. The compound of claim 24, wherein:
B is H, optionally substituted alkyl or phenyl;
n is an integer from 1 to 3;
R₁ to R₃ are H;
R₁₀ to R₁₈ and R₄₀ to R₄₄ are each independently H, halogen, or an optionally substituted moiety selected from carboxy, alkyl, alkoxy, alkoxy, aryloxy, sulfonate, thiol, alkylthiol, arylthiol, aryl, and heteroaryl;
R₂₀ and R₂₁ are each independently an optionally substituted alkyl, a carboxylated alkyl, or sulfonated alkyl; and
R₃₀ and R₃₁ are each optionally substituted alkyl;
provided that at least there are two sulfonates and a reactive moiety.

26. (canceled)

27. A compound having Formula VIII: 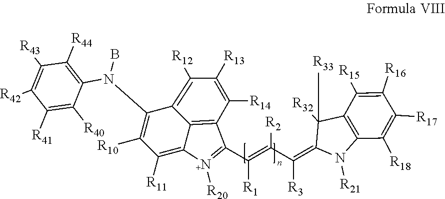
wherein:
B is H, or an optionally substituted alkyl, aryl, or heteroaryl;
n is an integer from 0 to 3;
R₁ to R₃ are each independently H, halogen, or an optionally substituted moiety selected from alkyl, aryl, thiol, amino, and heterocycle;
R₁₀ to R₁₈ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ and R₂₁ are each independently an optionally substituted alkyl, aryl, or heteroaryl; and
R₃₂ and R₃₃ are each independently an optionally substituted alkyl, aryl, or heteroaryl;
provided that at least there is a reactive moiety and a water soluble moiety.

28. The compound of claim 27, wherein:
B is H, or an optionally substituted alkyl or phenyl;
n is an integer from 1 to 3;
R₁ to R₃ are H;
R₁₀ to R₁₈ and R₄₀ to R₄₄ are each independently H, halogen, or an optionally substituted moiety selected from carboxy, alkyl, alkoxy, aryloxy, sulfonate, thiol, alkylthiol, arylthiol, aryl, and heteroaryl;
R₂₀ and R₂₁ are each independently optionally substituted alkyl, carboxylated alkyl or sulfonated alkyl; and
R₃₂ and R₃₃ are an optionally substituted alkyl;
provided that at least there are two sulfonates and a reactive moiety.

29. (canceled)

30. A compound having Formula IX: 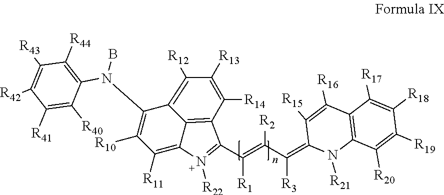
wherein:
B is H, optionally substituted alkyl, aryl, or heteroaryl;
n is an integer from 0 to 3;
R₁ to R₃ are each independently H, halogen, optionally substituted alkyl, aryl, thiol, amino, or heterocycle;
R₁₀ to R₂₀ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl; and
R₂₁ and R₂₂ are each independently optionally substituted alkyl, aryl, or heteroaryl;
provided that at least there is a reactive moiety.

31. The compound of claim 30, wherein:
B is H, optionally substituted alkyl or phenyl;
n is an integer from 1 to 3;
R₁ to R₃ are H;
R₁₀ to R₂₀ and R₄₀ to R₄₄ are each independently H, halogen, or an optionally substituted moiety selected from carboxy, alkyl, alkoxy, aryloxy, aryloxy, sulfonate, thiol, alkylthiol, arylthiol, aryl, and heteroaryl; and
R₂₁ and R₂₂ are each independently optionally substituted alkyl, carboxylated alkyl, or sulfonated alkyl;
provided that at least there is a reactive moiety.

32. (canceled)

33. A compound having Formula X: 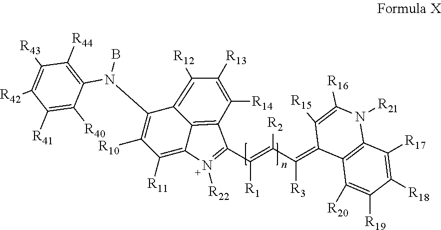
wherein:
B is H, optionally substituted alkyl, aryl, or heteroaryl;
n is an integer from 0 to 3;
R₁ to R₃ are each independently H, halogen, or an optionally substituted moiety selected from alkyl, aryl, thiol, amino, and heterocycle;
R₁₀ to R₂₀ and R₄₀ to R₄₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl; and
R₂₁ and R₂₂ are each independently optionally substituted alkyl, aryl, or heteroaryl;
provided that at least there is a reactive moiety.

34. The compound of claim 33, wherein:
B is H, an optionally substituted alkyl or phenyl;
n is an integer from 1 to 3;
R₁ to R₃ are H;
R₁₀ to R₂₀ and R₄₀ to R₄₄ are each independently H, halogen, or an optionally substituted moiety selected from carboxy, alkyl, alkoxy, aryloxy, sulfonate, thiol, alkylthiol, arylthiol, aryl, and heteroaryl; and
R₂₁ and R₂₂ are each independently an optionally substituted alkyl, carboxylated alkyl, or sulfonated alkyl;
provided that at least there are two sulfonates and a reactive moiety.

35. The compound of claim 33, wherein:
B is H, an optionally substituted alkyl or sulfonated alkyl;
n is 1 or 2;
R₁ to R₃ are H;
R₁₀ to R₂₀ and R₄₀ to R₄₄ are each independently H, halogen, or an optionally substituted moiety selected from alkyl, alkoxy, sulfonate, aryl, and heteroaryl; and
R₂₁ and R₂₂ are each independently carboxylated alkyl or sulfonated alkyl.

36. A compound having Formula XI:
{X—[CR₁—CR₂]ₙ—CR₃═Y}—CEP Formula XI
wherein:
n is an integer from 0 to 3;
X is an optionally substituted moiety selected from indolium, pyrrolium, oxazolium, thiazolium, imidazolium, pyridinium, and quinolinium;
Y is an optionally substituted moiety selected from indoline, pyrrole, oxazoline, thiazoline, imidazoline, pyridine, and quinoline;
R₁ to R₃ are each independently H, halogen, or an optionally substituted moiety selected from alkyl, aryl, thiol, amino and heterocycle; and
CEP is a phosphoramidite moiety;
provided that at least one of X and Y contains an optionally substituted arylamino or heteroarylamino group.

37. The compound of claim 36, X is an optionally substituted moiety selected from indolium and quinolinium;
Y is an optionally substituted moiety selected from indoline and quinoline;
R₁ to R₃ are H.

38. The compound of claim 36, wherein X has Formula II 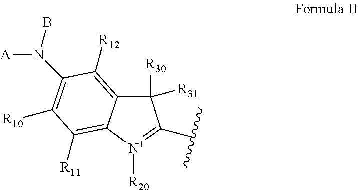
wherein:
A and B are each independently H, or an optionally substituted moiety selected from alkyl, aryl, and heteroaryl;
R₁₀ to R₁₂ are each independently H, halogen, or an optionally substituted carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ is an optionally substituted alkyl, aryl, or heteroaryl;
R₃₀ and R₃₁ are each independently optionally substituted alkyl, aryl, or heteroaryl provided that at least one of A and B is aryl or heteroaryl.

39. The compound of claim 38, wherein:
A and B are each independently H, or an optionally substituted alkyl or phenyl;
R₁₀ to R₁₂ are each independently H, halogen, or an optionally substituted moiety selected from carboxy, alkyl, alkoxy, aryloxy, thiol, alkylthiol, arylthiol, aryl, and heteroaryl;
R₂₀ is an optionally substituted alkyl, aryl, or heteroaryl; and
R₃₀ and R₃₁ are each independently optionally substituted alkyl or aryl.

40. The compound of claim 36, wherein X has Formula III 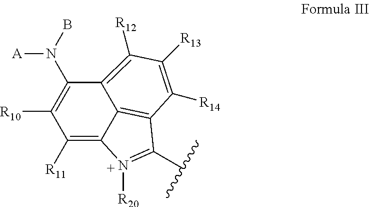
wherein:
A and B are each independently H, or an optionally substituted moiety selected from alkyl, aryl, and heteroaryl;
R₁₀ to R₁₄ are each independently H, halogen, or an optionally substituted moiety selected from carboxy, alkyl, alkoxy, aryloxy, thiol, alkylthiol, arylthiol, azido, cyano, amino, hydroxy, phosphonyl, sulfonyl, carbonyl, boryl, aryl, and heteroaryl;
R₂₀ is an optionally substituted alkyl, aryl, and heteroaryl;
provided that at least one of A and B is aryl or heteroaryl.

41. (canceled)

42. A dye-conjugate having Formula XII:
[Quencher]−[Sensing Moiety]−[Fluorophore] Formula XII
wherein:
Quencher is a quenching compound prepared from a compound of claim 1;
Sensing Moiety is a molecule capable of specifically binding to, or being cleaved by, an analyte of interest; and
Fluorophore is a fluorescent dye;
wherein the Sensing Moiety is bound or covalently linked to both the Quencher and the Fluorophore.

43. The dye-conjugate according to claim 42, wherein the Sensing Moiety comprises a peptide, a nucleotide, a protein, a nucleic acid or a carbohydrate.

44. The dye-conjugate according to claim 42, wherein the Fluorophore is a rhodamine dye, a cyanine dye, an oxazine dye, a BODIPY dye, a lanthanide complex dye, or a ruthenium complex dye.

45. The dye-conjugate according to claim 42, wherein the analyte of interest is a protease enzyme and the Sensing Moiety is a substrate for the enzyme.

46. The dye-conjugate according to claim 42, wherein the analyte of interest is a nucleotide capable of binding to the Sensing Moiety.

47. A method of detecting an analyte, comprising:
a) providing a sample that comprises a dye-conjugate having Formula XII
[Quencher]−[Sensing Moiety]−[Fluorophore] Formula XII
wherein:
Quencher is a quenching compound prepared from a compound of claim 1;
Sensing Moiety is a molecule capable of binding to, or being cleaved by, an analyte of interest;
Fluorophore is a fluorescent dye;
wherein:
the Sensing Moiety is configured to respond to a preselected environmental condition via changing a first separation distance between the Quencher and the Luminophore; and
the Sensing Moiety is bound or covalently linked to both the Quencher and the Fluorophore whereby FRET occurs between the Quencher and the Fluorophore at the first separation distance;
b) detecting a first luminescence response of said sample;
c) exposing the sample to an experimental environmental condition that is sufficient to change, or suspected to be sufficient to change, the first separation distance between the quencher and the Luminophore;
d) detecting a second luminescence response of said sample;
e) determining a difference between said first and second luminescence responses;
f) correlating said difference to a change in the separation distance between said Quencher and said Fluorophore; and
g) correlating said change in the separation distance between said Quencher and said Fluorophore with said experimental environmental condition.

48. An assay kit, comprising:
a) a dye-conjugate according to claim 42; and
b) a biological buffer.

49-52. (canceled)

53. A method of labeling a peptide, oligopeptide, or protein, the method comprising:
contacting a peptide, oligopeptide, or protein comprising a nucleophilic amino acid residue with a compound of claim 1 to produce a labeled peptide, oligopeptide, or protein.

54. (canceled)

55. A method of labeling a nucleic acid oligomer or polymer, the method comprising:
contacting a nucleic acid oligomer or polymer comprising a nucleophilic nucleic acid residue with a compound of claim 1 to produce a labeled nucleic acid oligomer or polymer.

56-60. (canceled)


SEQUENCE LISTING (4 sequences)

SEQ ID NO: 1 - protein, 10 aa, synthetic construct
  MOD_RES 1: note = Gamma amino butyric acid with N-terminal linked 3-(2-((1E,3E,5E)-7-((E)-1-(5-carboxypentyl)-3,3-dimethyl-5 -sulfoindolin-2-ylidene)hepta-1,3,5-trien-1-yl)-3,3-dimeth yl-5-(methyl(phenyl)amino)-3H-indol-1-ium-1-yl)propane-1-s ulfonate
  MOD_RES 3: note = 3-cyclohexyl alanine
  MOD_RES 4: note = Amino butyric acid
  MOD_RES 5: note = S-methyl cysteine
  MOD_RES 8: note = 2,4-diamino butyric acid with sidechain linked Cy7 dye
XPXXXHAXAK

SEQ ID NO: 2 - protein, 13 aa, synthetic construct
  MOD_RES 2: note = Glutamic acid with sidechain linked iFluor 750 dye
  MOD_RES 12: note = Lysine with sidechain linked 3-(2-((1E,3E,5E)-7-((E )-1-(5-carboxypentyl)-3,3-dimethyl-5-sulfoindolin-2-yliden e)hepta-1,3,5-trien-1-yl)-3,3-dimethyl-5-(methyl(phenyl)am ino)-3H-indol-1-ium-1-yl)propane-1-sulfonate
RXVSFNFPQITXR

SEQ ID NO: 3 - protein, 10 aa, synthetic construct
  MOD_RES 1: note = Gamma amino butyric acid
  MOD_RES 3: note = 3-cyclohexyl alanine
  MOD_RES 4: note = Amino butyric acid
  MOD_RES 5: note = S-methyl cysteine
  MOD_RES 8: note = 2,4-diamino butyric acid with sidechain linked Cy7 dye
XPXXXHAXAK

SEQ ID NO: 4 - protein, 10 aa, synthetic construct
  MOD_RES 1: note = Gamma amino butyric acid with N-terminal linked 3-(2-((1E,3E,5E)-7-((E)-1-(5-carboxypentyl)-3,3-dimethyl-5 -sulfoindolin-2-ylidene)hepta-1,3,5-trien-1-yl)-3,3-dimeth yl-5-(methyl(phenyl)amino)-3H-indol-1-ium-1-yl)propane-1-s ulfonate
  MOD_RES 3: note = 3-cyclohexyl alanine
  MOD_RES 4: note = Amino butyric acid
  MOD_RES 5: note = S-methyl cysteine
  MOD_RES 8: note = 2,4-diamino butyric acid with sidechain linked Cy7 dye
  MOD_RES 10: note = Lysine with sidechain linked 3-(2-((1E,3E,5E)-7-((E )-1-(5-carboxypentyl)-3,3-dimethyl-5-sulfoindolin-2-yliden e)hepta-1,3,5-trien-1-yl)-3,3-dimethyl-5-(methyl(phenyl)am ino)-3H-indol-1-ium-1-yl)propane-1-sulfonate
XPXXXHAXAX